(19) United States
(12) Patent Application Publication
(10) Pub. No.: US 2022/0202867 A1
(43) Pub. Date: Jun. 30, 2022

(54) TREATMENT OF KIDNEY DISEASE IN SUBJECTS WITH KIDNEY AND/OR URINARY TRACT ANOMALIES

(71) Applicants: Timothy Bertram, George Town (KY); Deepak Jain, Winston-Salem, NC (US)

(72) Inventors: Timothy Bertram, George Town (KY); Deepak Jain, Winston-Salem, NC (US)

(21) Appl. No.: 17/605,714

(22) PCT Filed: May 1, 2020
(86) PCT No.: PCT/US2020/031093

§ 371 Date: Oct. 22, 2021

Related U.S. Application Data
(60) Provisional application No. 62/842,150, filed on May 2, 2019.

(51) Int. Cl. A61K 35/22; A61P 13/12

(52) U.S. Cl. CPC A61K 35/22; A61P 13/12

(57) ABSTRACT

Provided herein arm, inter alia, methods, cell populations, and compositions for treating chronic kidney disease in subjects with a congenital anomaly of a kidney and/or urinary tract.

### FIELD OF THE INVENTION

The present invention relates to, inter alia, methods, compositions, and cell populations for treating subjects with kidney disease.

### BACKGROUND

Anomalies of the kidney, such as congenital anomalies of the kidney and urinary tract (CAKUT) and/or acquired anomalies, can lead to renal disorders including chronic and end-stage kidney disease. CAKUT constitute approximately 20 to 30 percent of all anomalies identified in the prenatal period. See Queisser-Luft et al. (2002) Malformations in newborn: results based on 30,940 infants and fetuses from the Mainz congenital birth defect monitoring system (1990-1998). 2002; 266(3):163, the entire content of which is incorporated herein by reference.

### BRIEF SUMMARY

Provided herein are, inter alia, methods, cell populations, and compositions for treating kidney disease in subjects with a congenital anomaly of a kidney and/or urinary tract.

In an aspect, provided herein is a method of treating kidney disease in a subject who has chronic kidney disease (CKD), the method comprising administering to the subject an effective amount of (i) a bioactive renal cell population; (ii) vesicles secreted by the renal cell population; and/or (iii) spheroids comprising the renal cell population and at least one non-renal cell population, wherein the subject has an anomaly of a kidney and/or urinary tract.

In embodiments, the subject has an anomaly of a kidney. In embodiments, the subject has an anomaly of a urinary tract. In embodiments, the subject has an anomaly of a kidney and urinary tract. In embodiments, an anomaly is acquired before birth. In embodiments, an anomaly is acquired after birth. In embodiments, an anomaly is a congenital anomaly. In embodiments, the subject has a congenital anomaly of a kidney. In embodiments, the subject has a congenital anomaly of a urinary tract. In embodiments, the subject has a congenital anomaly of a kidney and urinary tract. As used herein, a “congenital” anomaly is an abnormality that is present at or before birth. In embodiments, a congenital anomaly worsens or gives rise to additional abnormalities after birth.

In an aspect, provided herein is a method of treating kidney disease in a subject who has an anomaly of a kidney and/or urinary tract, the method comprising, consisting essentially of, or consisting of administering to the subject an effective amount of (i) a bioactive renal cell population; (ii) one or more products secreted by the renal cell population; and/or (iii) spheroids comprising the renal cell population and at least one other cell population.

In an aspect, provided herein is a method of treating kidney disease in a subject who has an anomaly of a kidney and/or urinary tract, the method comprising administering to the subject an effective amount of (i) a bioactive renal cell population; (ii) one or more products (such as vesicles) secreted by the renal cell population; and/or (iii) spheroids comprising the renal cell population and at least one other cell population, such as a non-renal cell population.

In an aspect, provided herein is a method of treating kidney disease in a subject who has an anomaly of a kidney and/or urinary tract, the method comprising administering to the subject an effective amount of (i) a bioactive renal cell population; (ii) vesicles secreted by the renal cell population; or (iii) spheroids comprising the renal cell population and at least one non-renal cell population.

In an aspect, provided herein is a method of treating kidney disease in a subject who has an anomaly of a kidney and/or urinary tract, the method comprising administering to the subject an effective amount of (i) a bioactive renal cell population; (ii) vesicles secreted by the renal cell population; and (iii) spheroids comprising the renal cell population and at least one non-renal cell population.

In an aspect, provided herein is a method of treating kidney disease in a subject who has an anomaly of a kidney and/or urinary tract, the method comprising administering to the subject an effective amount of a composition comprising a bioactive renal cell population. In embodiments, the composition further comprises vesicles secreted by the renal cell population. In embodiments, the composition further comprises spheroids comprising the renal cell population and at least one non-renal cell population.

In an aspect, provided herein is a method of treating kidney disease in a subject who has an anomaly of a kidney and/or urinary tract, the method comprising administering to the subject an effective amount of vesicles secreted by a renal cell population.

In an aspect, provided herein is method of treating kidney disease in a subject who has an anomaly of a kidney and/or urinary tract, the method comprising administering to the subject an effective amount of spheroids comprising a renal cell population and at least one non-renal cell population.

In an aspect, provided herein is a bioactive renal cell population and uses thereof for treating kidney disease in a subject who has an anomaly of a kidney and/or urinary tract.

In an aspect, provided herein are products (such as vesicles) secreted by a bioactive renal cell population and uses thereof for treating kidney disease in a subject who has an anomaly of a kidney and/or urinary tract.

In an aspect, provided herein are spheroids comprising a bioactive renal cell population and uses thereof for treating kidney disease in a subject who has an anomaly of a kidney and/or urinary tract.

### BRIEF DESCRIPTION OF THE DRAWINGS

FIG. 1 is a graph showing estimated glomerular filtration rate (eGFR) pre- and post-REACT treatment.

FIG. 2 is a graph showing serum creatinine pre- and post-REACT treatment.

FIG. 3 is a photograph of a REACT product delivery system.

FIG. 4 is a photograph of a REACT shipping container.

FIG. 5 is study design flow diagram.

FIG. 6 is a flow diagram of a non-limiting example of an overall NKA manufacturing process.

FIG. 7 A-D are flow diagrams providing further details of the non-limiting example process depicted in FIG. 6.

FIG. 8 is a graph showing improvement in renal function as measured by eGFR in a patient receiving REACT treatment for kidney disease resulting from CAKUT; star shows patient's initial renal function before effect of CAKUT; solid gray line (plotted −1 to 0 months relative to injection) patient's declining renal function as measured by eGFR pre-REACT injection; broken black line (plotted at 0 to 3 months relative to injection), patient's eGFR following REACT injection.

FIG. 9 is a graph showing improvement in renal function as measured by albumin-to creatinine ratio in a patient receiving REACT treatment for kidney disease resulting from CAKUT.

### DETAILED DESCRIPTION

Reference is made herein to certain embodiments and examples encompassed by the invention. While the invention will be described in conjunction with exemplary embodiments, it will be understood that they are not intended to limit the invention to those embodiments. On the contrary, the invention is intended to cover all alternatives, modifications, and equivalents which may be included within the scope of the present invention as defined by the claims. One skilled in the art will recognize many methods and materials similar or equivalent to those described herein, which could be used in the practice of the present invention. The present invention is in no way limited to the methods and materials described.

All references cited throughout the disclosure are expressly incorporated by reference herein in their entirety. In the event that one or more of the incorporated literature, patents, and similar materials differs from or contradicts this application, including but not limited to defined terms, term usage, described techniques, or the like, this application controls.

Unless defined otherwise, technical and scientific terms used herein have the same meaning as commonly understood by one of ordinary skill in the art to which this invention belongs. One skilled in the art will recognize many methods and materials similar or equivalent to those described herein, which could be used in the practice of the present invention. Indeed, the present invention is in no way limited to the methods and materials described.

As used herein, the term “about” in the context of a numerical value or range means±10% of the numerical value or range recited or claimed, unless the context requires a more limited range.

In the descriptions herein and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C” “one or more of A, B, and C” “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

It is understood that where a parameter range is provided, all integers within that range, and tenths thereof, are also provided by the invention. For example, “0.2-5 mg” is a disclosure of 0.2 mg, 0.3 mg, 0.4 mg, 0.5 mg, 0.6 mg etc. up to and including 5.0 mg.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

As used herein, the singular forms “a,” “an,” and “the” include the plural reference unless the context clearly dictates otherwise.

As used herein, “treating” encompasses, e.g., inhibition, regression, or stasis of the progression of a disorder. Treating also encompasses the prevention or amelioration of any symptom or symptoms of the disorder. As used herein, “inhibition” of disease progression or a disease complication in a subject means preventing or reducing the disease progression and/or disease complication in the subject.

As used herein, a “symptom” associated with a disorder includes any clinical or laboratory manifestation associated with the disorder, and is not limited to what the subject can feel or observe.

As used herein, “effective” when referring to an amount of a therapeutic agent refers to the quantity of the agent that is sufficient to yield a desired therapeutic response without undue adverse side effects (such as toxicity, irritation, or allergic response) commensurate with a reasonable benefit/risk ratio when used in the manner of this disclosure.

The term “bioactive renal cells” or “BRCs” as used herein refers to renal cells having one or more of the following properties when administered into the kidney of a subject: capability to reduce (e.g., slow or halt) the worsening or progression of chronic kidney disease or a symptom thereof, capability to enhance renal function, capability to affect (improve) renal homeostasis, and capability to promote healing, repair and/or regeneration of renal tissue or kidney. In embodiments, these cells may include functional tubular cells (e.g., based on improvements in creatinine excretion and protein retention), glomerular cells (e.g., based on improvement in protein retention), vascular cells and other cells of the corticomedullary junction. In embodiments, BRCs are obtained from isolation and expansion of renal cells from kidney tissue. In embodiments, BRCs are obtained from isolation and expansion of renal cells from kidney tissue using methods that select for bioactive cells. In embodiments, the BRCs have a regenerative effect on the kidney. In embodiments, BRCs comprise, consist essentially of, or consist of selected renal cells (SRCs). In embodiments, BRCs are SRCs.

In embodiments, SRCs are cells obtained from isolation and expansion of renal cells from a suitable renal tissue source, wherein the SRCs contain a greater percentage of one or more cell types and lacks or has a lower percentage of one or more other cell types, as compared to a starting kidney cell population. In embodiments, the SRCs contain an increased proportion of BRCs compared to a starting kidney cell population. In embodiments, an SRC population is an isolated population of kidney cells enriched for specific bioactive components and/or cell types and/or depleted of specific inactive and/or undesired components or cell types for use in the treatment of kidney disease, i.e., providing stabilization and/or improvement and/or regeneration of kidney function. SRCs provide superior therapeutic and regenerative outcomes as compared with the starting population. In embodiments, SRCs are obtained from the patient's renal cortical tissue via a kidney biopsy. In embodiments, SRCs are selected (e.g., by fluorescence-activated cell sorting or “FACS”) based on their expression of one or more markers. In embodiments, SRCs are depleted (e.g., by fluorescence-activated cell sorting or “FACS”) of one or more cell types based on the expression of one or more markers on the cell types. In embodiments, SRCs are selected from a population of bioactive renal cells. In embodiments, SRCs are selected by density gradient separation of expanded renal cells. In embodiments, SRCs are selected by separation of expanded renal cells by centrifugation across a density boundary, barrier, or interface, or single step discontinuous step gradient separation. In embodiments, SRCs are selected by continuous or discontinuous density gradient separation of expanded renal cells that have been cultured under hypoxic conditions. In embodiments, SRCs are selected by density gradient separation of expanded renal cells that have been cultured under hypoxic conditions for at least about 8, 12, 16, 20, or 24 hours. In embodiments, SRCs are selected by separation by centrifugation across a density boundary, barrier, or interface of expanded renal cells that have been cultured under hypoxic conditions. In embodiments, SRCs are selected by separation of expanded renal cells that have been cultured under hypoxic conditions for at least about 8, 12, 16, 20, or 24 hours by centrifugation across a density boundary, barrier, or interface (e.g., single-step discontinuous density gradient separation). In embodiments, SRCs are composed primarily of renal tubular cells. In embodiments, other parenchymal (e.g., vascular) and stromal (e.g., collecting duct) cells may be present in SRCs. In embodiments, less than about 10%, 9%, 8%, 7%, 6%, 5%, 4%, 3%, 2%, or 1% of the cells in a population of SRCs are vascular cells. In embodiments, less than about 10%, 9%, 8%, 7%, 6%, 5%, 4%, 3%, 2%, or 1% of the cells in a population of SRCs are collecting duct cells. In embodiments, less than about 10%, 9%, 8%, 7%, 6%, 5%, 4%, 3%, 2%, or 1% of the cells in a population of SRCs are vascular or collecting duct cells.

The term “spheroid” refers to an aggregate or assembly of cells cultured to allow 3-dimensional growth as opposed to growth as a monolayer. It is noted that the term “spheroid” does not imply that the aggregate is a geometric sphere. In embodiments, the aggregate may be highly organized with a well defined morphology or the aggregate may be an unorganized mass. In embodiments, a spheroid may include a single cell type or more than one cell type. In embodiments, the cells may be primary isolates, or a permanent cell line, or a combination of the two. In embodiments, the spheroids (e.g., cellular aggregates or organoids) are formed in a spinner flask. In embodiments, the spheroids (e.g., cellular aggregates or organoids) are formed in a 3-dimensional matrix.

The term “native organ” shall mean the organ of a living subject. In embodiments, the subject may be healthy or unhealthy. In embodiments, an unhealthy subject may have a disease associated with that particular organ.

The term “native kidney” shall mean the kidney of a living subject. In embodiments, the subject may be healthy or unhealthy. In embodiments, an unhealthy subject may have a kidney disease. In embodiments, an unhealthy subject may have an anomaly of a kidney and/or urinary tract.

Provided herein are cell types and populations of cells (such as SRCs) that provide a benefit to a native organ, such as the kidney. In embodiments, the benefit includes a halt or slowing of the progression (e.g., the worsening of one or more symptoms) of chronic kidney disease. In embodiments, the benefit includes a regenerative effect, e.g., reduction in a symptom of chronic kidney disease and/or an improvement in native kidney function. In embodiments, the benefit includes, without limitation, a reduction in the degree of injury to a native organ or an improvement in, restoration of, or stabilization of a native organ function or structure. Renal injury may be, e.g., in the form of fibrosis, inflammation, glomerular hypertrophy, atrophy, etc.

In embodiments, an enriched cell population or preparation is a cell population derived from a starting organ cell population (e.g., an unfractionated, heterogeneous cell population from a kidney) that contains a greater percentage of a specific cell type than the percentage of that cell type in the starting population. For example, a starting kidney cell population can be enriched for a first, a second, a third, a fourth, a fifth, and so on, cell type of interest.

The term “hypoxic” culture conditions as used herein refers to culture conditions in which cells are subjected to a reduction in available oxygen levels in the culture system relative to standard culture conditions in which cells are cultured at atmospheric oxygen levels (about 21%). Non-hypoxic conditions are referred to herein as normal or normoxic culture conditions.

Included herein are compositions comprising a biomaterial and one or more cell types. In embodiments, the biomaterial is a natural or synthetic biocompatible material that is suitable for introduction into living tissue supporting cells in a viable state. A natural biomaterial is a material that is made by or originates from a living system. Synthetic biomaterials are materials which are not made by or do not originate from a living system. In embodiments, a biomaterial disclosed herein may be a combination of natural and synthetic biocompatible materials. As used herein, biomaterials include (but are not limited to), for example, polymeric matrices and scaffolds. Those of ordinary skill in the art will appreciate that the biomaterial(s) may be configured in various forms, for example, as porous foam, gels, liquids, beads, solids, and may comprise one or more natural or synthetic biocompatible materials. In embodiments, the biomaterial is the liquid form of a solution that is capable of becoming a hydrogel. In embodiments, the biomaterials is a hydrogel that is capable of becoming a liquid.

The term “kidney disease” as used herein includes disorders associated with any stage or degree of acute or chronic renal disease (e.g., acute or chronic renal failure) that results in a reduction or loss of the kidney's ability to perform the function of blood filtration and elimination of excess fluid, electrolytes, and wastes from the blood. In embodiments, kidney disease also includes endocrine dysfunctions such as anemia (erythropoietin-deficiency), and mineral imbalance (Vitamin D deficiency). In embodiments, kidney disease may originate in the kidney or may be secondary to a variety of conditions, including (but not limited to) congenital anomalies of the kidney and urinary tract (CAKUT), vesicoureteral reflux, heart failure, hypertension, diabetes, autoimmune disease, or liver disease. In embodiments, kidney disease may be a condition of chronic renal failure that develops after an acute injury to the kidney. For example, injury to the kidney by ischemia and/or exposure to toxicants may cause acute renal failure; incomplete recovery after acute kidney injury may lead to the development of chronic renal failure.

In embodiments, the term “treatment” may refer to therapeutic treatment and/or prophylactic or preventative measures for kidney disease, anemia, tubular transport deficiency, or glomerular filtration deficiency wherein the object is to reverse, prevent or slow down (lessen) the targeted disorder. Those in need of treatment include those already having a kidney disease, anemia, tubular transport deficiency, or glomerular filtration deficiency as well as those prone to having a kidney disease, anemia, tubular transport deficiency, or glomerular filtration deficiency or those in whom the kidney disease, anemia, tubular transport deficiency, or glomerular filtration deficiency is to be prevented. In embodiments, a subject in need of treatment comprises a congenital anomaly of a kidney and/or urinary tract. The term “treatment” as used herein includes the stabilization and/or improvement of kidney function.

Included herein are constructs or formulations comprising one or more cell types (e.g., a cell population such as SRCs) deposited on or in a surface of a scaffold or matrix made up of one or more synthetic or naturally-occurring biocompatible materials. In embodiments, the one or more cell populations may be coated with, deposited on, embedded in, attached to, seeded, or entrapped in a biomaterial made up of one or more synthetic or naturally-occurring biocompatible biomaterials, polymers, proteins, or peptides. In embodiments, the one or more cell populations may be combined with a biomaterial or scaffold or matrix in vitro or in vivo. In embodiments, the one or more biomaterials used to generate the construct or formulation may be selected to direct, facilitate, or permit dispersion and/or integration of the cellular components of the construct with the endogenous host tissue, or to direct, facilitate, or permit the survival, engraftment, tolerance, or functional performance of the cellular components of the construct or formulation.

The term “Neo-Kidney Augment (NKA)” refers to a bioactive cell formulation which is an injectable product composed of autologous, homologous SRCs formulated in a biomaterial comprised of a gelatin-based hydrogel. The term “Advance Cell Therapy (ACT)” refers to treatment with NKA.

In embodiments, a subject is a living animal. In embodiments, a subject is a mammal such as a dog, cat, horse, rabbit, zoo animal, cow, pig, sheep, goat, camel, mouse, rat, or guinea pig. In embodiments, a subject is a primate such as a human, a chimpanzee, an orangutan, a monkey, or a baboon. In embodiments, a subject is a human. In embodiments, a subject is a patient, eligible for treatment, who is experiencing or has experienced one or more signs, symptoms, or other indicators of a kidney disease. Such subjects include without limitation subjects who are newly diagnosed or previously diagnosed and are now experiencing a recurrence or relapse, or are at risk for a kidney disease, no matter the cause. In embodiments, the subject may have been previously treated for a kidney disease, or not so treated. In embodiments, a subject has a congenital anomaly of a kidney and/or urinary tract. In embodiments, a subject is a human with congenital anomalies of the kidney and urinary tract. In embodiments, a subject is experiencing or has experienced one or more signs, symptoms, or other indicators of an organ-related disease, such as kidney disease, anemia, or erythropoietin (EPO) deficiency. In embodiments, the subject does not have diabetes. In embodiments, the subject does not have Type I diabetes. In embodiments, the subject does not have Type II diabetes.

Congenital anomalies of the kidney and urinary tract (CAKUT) includes a family of diseases of various anatomic spectrum, including renal anomalies, and anomalies of the bladder and urethra. In embodiments, the term “CAKUT” refers to one congenital abnormality (e.g., when referring to a subject who has CAKUT). In embodiments, the term CAKUT refers to more than one congenital abnormality (e.g., when referring to a subject who has CAKUT). In embodiments, a subject with CAKUT has one or more abnormalities of the kidney, bladder, and/or urethra. In embodiments, a subject with CAKUT has an abnormality in one or two kidneys. In embodiments, a subject with CAKUT has an abnormality in the urethra. In embodiments, the CAKUT has resulted from a genetic mutation or abnormality. In embodiments, the CAKUT has resulted from an environmental factor. In embodiments, a subject with CAKUT has an abnormality in the bladder. Non-limiting descriptions relating to CAKUT are provided in Ristoska-Bojkovska (2017) Pril (Makedon Akad Nauk Umet Odd Med Nauki) 38(1):59-62; and Rodriguez (2014) Fetal Pediatr Pathol. 33(5-6):293-320, the entire contents of each of which are incorporated herein by reference. In embodiments, a subject who has CAKUT does not have diabetes. In embodiments, a subject who has CAKUT does not have Type I diabetes. In embodiments, a subject who has CAKUT does not have Type II diabetes.

CAKUT constitute approximately 20 to 30 percent of all anomalies identified in the prenatal period. See Queisser-Luft et al. (2002) Malformations in newborn: results based on 30,940 infants and fetuses from the Mainz congenital birth defect monitoring system (1990-1998). Spranger J Arch Gynecol Obstet. 2002; 266(3):163, the entire content of which is incorporated herein by reference. In embodiments, defects can be bilateral or unilateral, and different defects often coexist in an individual child.

In embodiments, CAKUT represent a broad range of disorders that result from abnormal embryogenic renal development due to renal parenchymal malformations, abnormalities in renal migration, or abnormalities in the developing collecting system. In embodiments, CAKUT represent a broad range of disorders and are the result of abnormal renal developmental processes. In embodiments, malformation of the renal parenchyma results in failure of normal nephron development, as seen in renal dysplasia, rheumatoid arthritis (RA), renal tubular dysgenesis, and some types of nephronophthisis. Without being bound by any scientific theory, investigation utilizing molecular genetics has demonstrated that renal malformation results from defects in genes that encode signaling and transcription factors. In embodiments, environmental factors, such as prenatal exposure to teratogens, can also disrupt renal morphogenesis resulting in CAKUT. In embodiments, abnormalities comprise abnormal embryonic migration of the kidneys, as seen in renal ectopy (e.g., pelvic kidney), and fusion anomalies, such as horseshoe kidney. In embodiments, abnormalities of the developing urinary collecting system, as seen in duplicate collecting systems, posterior urethral valves, and ureteropelvic junction obstruction may lead to CKD/ESRD. Renal dysplasia may be unilateral or bilateral and occurs in two to four per 1000 births. The male-to-female ratio for bilateral renal dysplasia is 1.3:1, and for unilateral dysplasia is 1.9:1 Because CAKUT play a causative role in 30 to 50 percent of cases of end-stage renal disease (ESRD) in children (Seikaly et al. 2003 Chronic renal insufficiency in children: the 2001 Annual Report Pediatr Nephrol. 18(8):796), in embodiments it is important to diagnose these anomalies and initiate therapy to minimize renal damage, prevent or delay the onset of ESRD, and provide supportive care to avoid complications of ESRD. Patients with malformations involving a reduction in kidney numbers or size are most likely to have a poor renal prognosis (Sanna-Cherchi et al. 2009 Renal outcome in patients with congenital anomalies of the kidney and urinary tract. Kidney Int. 76(5):528). In embodiments, by 30 years of age, most patients will have dialysis.

The risk for dialysis is significantly higher for patients with a solitary kidney or with renal hypodysplasia associated with posterior urethral valves compared to patients with unilateral or bilateral renal hypodysplasia, or multicystic or horseshoe kidney. In embodiments, sub-clinical defects of the solitary kidney maybe responsible for a poorer prognosis compared to more benign forms of CAKUT. In embodiments, and without being limited by any scientific theory, children with a solitary kidney are at risk for long-term CKD, which is thought to be due to glomerular hyperfiltration. In embodiments, about one-third of patients can have evidence of renal injury defined as proteinuria (e.g., urine protein to creatinine ratio >0.2 mg/mg [>22.6 mg/mmol in children greater than two years of age]), hypertension (e.g., blood pressure ≥95^{th} percentile for age, gender, and height), elevated estimated creatinine clearance based on serum creatine and Schwartz equation, or the use of medication for renal protection (e.g., angiotensin-converting enzyme inhibitors).

In embodiments, renal dysplasia may be discovered during routine antenatal screening or postnatally when renal ultrasonography is performed in a dysmorphic infant. In embodiments, bilateral dysplasia is likely to be diagnosed earlier than unilateral dysplasia especially if oligohydramnios is present. In embodiments, renal ultrasound features include increased echogenicity as a result of abnormal renal parenchymal tissue, poor corticomedullary differentiation, and parenchymal cysts.

In embodiments, infants with bilateral dysplasia may have impaired renal function at birth, and subsequent progressive renal failure may occur. In embodiments, associated urological findings include abnormalities of the renal pelvis, calyces (e.g., congenital hydronephrosis), and ureters e.g., duplicating collecting system megaureter, ureteral stenosis, and vesicoureteral reflux [VUR]. In embodiments, as a result, symptomatic presentation may occur due to complications associated with these urological anomalies, including urinary tract infection (UTI), hematuria, fever, and abdominal pain.

In embodiments, because of the frequent association of renal dysplasia with a collecting system anomaly, voiding cystourethrography may be considered in patients with renal dysplasia with or without a UTI. In embodiments, if there is an associated urological abnormality such as VUR in the normal contralateral kidney, children with unilateral renal dysplasia may be at increased risk of long-term sequelae of renal scarring from recurrent UTI. In embodiments, a DMSA radionuclide scan can provide further information on the differential function of each kidney. In embodiments, multicystic dysplastic kidney (MCDK) typically has no viable functional renal tissue and, therefore, no detectable renal blood flow or renal function. However, in embodiments, there may be rare variations of segmental dysplasia. In embodiments, imaging studies may be useful in defining baseline renal function and risk of future renal damage and the ability to regenerate normal functioning renal parenchyma.

The term “sample” or “patient sample” or “biological sample” shall generally include any biological sample obtained from a subject or patient, body fluid, body tissue, cell line, tissue culture, or other source. The term includes tissue biopsies such as, for example, kidney biopsies. The term includes cultured cells such as, for example, cultured mammalian kidney cells. Methods for obtaining tissue biopsies and cultured cells from mammals are well known in the art. In embodiments, a sample may originate from various sources in a mammalian subject including, without limitation, blood, semen, serum, urine, bone marrow, mucosa, tissue, etc.

The term “control sample” refers a negative or positive control sample in which a negative or positive result is expected to help correlate a result in the test sample. In embodiments, a suitable control sample includes, without limitation, a sample known to exhibit indicators characteristic of normal kidney function, a sample obtained from a subject known not to have kidney disease, and a sample obtained from a subject known to have kidney disease. In embodiments, a control sample may be a sample obtained from a subject prior to being treated by a method provided herein. In embodiments, a control sample may be a test sample obtained from a subject known to have any type or stage of kidney disease, and a sample from a subject known not to have any type or stage of kidney disease. In embodiments, a control sample may be a normal healthy matched control. Those of skill in the art will appreciate other control samples suitable for use.

Provided herein are, inter alia, methods and compositions for treating chronic kidney disease in subjects with an anomaly of a kidney and/or urinary tract (e.g., a subject who has CAKUT).

In an aspect, provided herein is a method of treating kidney disease in a subject who has chronic kidney disease, the method comprising, consisting essentially of, or consisting of administering to the subject an effective amount of (i) a bioactive renal cell population; (ii) vesicles secreted by the renal cell population; and/or (iii) spheroids comprising the renal cell population and at least one non-renal cell population, wherein the subject has an anomaly of a kidney and/or urinary tract. In embodiments, the subject has an anomaly of a kidney.

In an aspect, provided herein is a method of treating kidney disease in a subject who has an anomaly of a kidney and/or urinary tract, the method comprising, consisting essentially of, or consisting of administering to the subject an effective amount of (i) a bioactive renal cell population; (ii) one or more products secreted by the renal cell population; and/or (iii) spheroids comprising the renal cell population and at least one other cell population.

In an aspect, provided herein is a method of treating kidney disease in a subject who has an anomaly of a kidney and/or urinary tract, the method comprising administering to the subject an effective amount of (i) a bioactive renal cell population; (ii) one or more products (such as vesicles) secreted by the renal cell population; and/or (iii) spheroids comprising the renal cell population and at least one other cell population, such as a non-renal cell population.

In an aspect, provided herein is a method of treating kidney disease in a subject who has an anomaly of a kidney and/or urinary tract, the method comprising administering to the subject an effective amount of (i) a bioactive renal cell population; (ii) vesicles secreted by the renal cell population; or (iii) spheroids comprising the renal cell population and at least one non-renal cell population.

In an aspect, provided herein is a method of treating kidney disease in a subject who has an anomaly of a kidney and/or urinary tract, the method comprising administering to the subject an effective amount of (i) a bioactive renal cell population; (ii) vesicles secreted by the renal cell population; and (iii) spheroids comprising the renal cell population and at least one non-renal cell population.

In an aspect, provided herein is a method of treating kidney disease in a subject who has an anomaly of a kidney and/or urinary tract, the method comprising administering to the subject an effective amount of a composition comprising a bioactive renal cell population. In embodiments, the composition further comprises vesicles secreted by the renal cell population. In embodiments, the composition further comprises spheroids comprising the renal cell population and at least one non-renal cell population.

In an aspect, provided herein is a method of treating kidney disease in a subject who has an anomaly of a kidney and/or urinary tract, the method comprising administering to the subject an effective amount of vesicles secreted by the renal cell population.

In an aspect, provided herein is a method of treating kidney disease in a subject who has an anomaly of a kidney and/or urinary tract, the method comprising administering to the subject an effective amount of spheroids comprising the renal cell population and at least one non-renal cell population.

In an aspect, provided herein is a bioactive renal cell population and uses thereof for treating kidney disease in a subject who has an anomaly of a kidney and/or urinary tract.

In an aspect, provided herein are products (such as vesicles) secreted by a bioactive renal cell population and uses thereof for treating kidney disease in a subject who has an anomaly of a kidney and/or urinary tract.

In an aspect, provided herein are spheroids comprising a bioactive renal cell population and uses thereof for treating kidney disease in a subject who has an anomaly of a kidney and/or urinary tract.

In embodiments, the subject has an anomaly of a urinary tract. In embodiments, the subject has an anomaly of a kidney and a urinary tract. In embodiments, the anomaly is acquired before birth. In embodiments, the anomaly is acquired after birth. In embodiments, the anomaly is a congenital anomaly. In embodiments, the subject has a congenital anomaly of a kidney. In embodiments, the subject has a congenital anomaly of the urinary tract. In embodiments, the subject has a congenital anomaly of a kidney and a urinary tract. In embodiments, a congenital anomaly worsens or gives rise to additional abnormalities after birth. In embodiments, a subject has an abnormality in one kidney. In embodiments, a subject has one or more abnormalities in each kidney. In embodiments, the subject has an abnormality in the urinary tract, wherein the abnormality is in the urethra. In embodiments, the subject has an abnormality in the urinary tract, wherein the abnormality is in the bladder. In embodiments, the subject has an abnormality in the urinary tract, wherein the abnormality is in a ureter. In embodiments, an anomaly is present at birth, but does not manifest or show symptoms until after birth.

In embodiments, the kidney disease is CKD. In embodiments, the subject has CKD from anomalies (e.g. congenitally) of the kidney and urinary tract.

In embodiments, the anomaly comprises a congenital anomaly. In embodiments, the subject has CAKUT.

In embodiments, the anomaly is a morphological anomaly. In embodiments, the subject has an abnormally developed kidney.

In embodiments, the subject has or has had primary vesicoureteral reflux, reflux nephropathy, renal scaring, or renal hypodysplasia. In embodiments, the subject has or has had reflux nephropathy. In embodiments, the subject has or has had renal scaring. In embodiments, the subject has or has had renal hypodysplasia.

In embodiments, the subject is predisposed to urinary tract infections. In embodiments, the subject has had at least about 1, 2, 3, 4, 5, 6, 7, 8, 9, or 10 urinary tract infections.

In embodiments, the subject has hypertension or proteinuria.

In embodiments, the subject has had post-antireflux surgery.

In embodiments, the subject has a glomerular filtration rate (GFR) of less than 90 mL/min/1.73 m², microalbuminuria, or macroalbuminuria. In embodiments, the subject has a GFR of less than 80 mL/min/1.73 m². In embodiments, the subject has a GFR of less than 70 mL/min/1.73 m². In embodiments, the subject has a GFR of less than 60 mL/min/1.73 m². In embodiments, the subject has a GFR of less than 50 mL/min/1.73 m². In embodiments, the subject has a GFR of less than 40 mL/min/1.73 m². In embodiments, the subject has a GFR of less than 30 mL/min/1.73 m². In embodiments, the subject has a GFR of at least 10 mL/min/1.73 m². In embodiments, the subject has a GFR of at least 15 mL/min/1.73 m². In embodiments, the subject has a GFR of lat least 20 mL/min/1.73 m². In embodiments, the subject has a GFR of at least 30 mL/min/1.73 m². In embodiments, the subject has a GFR of from 10, 15, 20, 25 or 30 mL/min/1.73 m² to 50, 60, 70, 80 or 90 mL/min/1.73 m². In embodiments, the GFR is the estimated GFR (eGFR). In embodiments, the subject has microalbuminuria. In embodiments, the subject has macroalbuminuria.

In embodiments, the subject is less than 18 years old. In embodiments, the subject is less than 60 years old. In embodiments, the subject is less than 50 years old. In embodiments, the subject is less than 40 years old. In embodiments, the subject is less than 35 years old. In embodiments, the subject is less than 30 years old. In embodiments, the subject is less than 25 years old. In embodiments, the subject is less than 20 years old. In embodiments, the subject is from 1 to 16 years old. In embodiments, the subject is from 1, 2, 3, 4, 5, 6, 7, 8, 9, or 10 to 20, 25, 30, 35, or 40 years old.

In embodiments, the subject is from 20, 25, 30, 35, or 40 to 50, 55, 65, 70, 75, 80, 85, 90, 95, or 100 years old. In embodiments, the subject is at least 50 years old. In embodiments, the subject is at least 55 years old. In embodiments, the subject is at least 60 years old. In embodiments, the subject is at least 65 years old. In embodiments, the subject is at least 70 years old.

In embodiments, the subject has a Renal Parenchymal Malformation.

In embodiments, the subject has a ureteral duplication, a ureteropelvic junction obstruction, renal agenesis, vesicoureteral reflux, renal dysplasia, renal hypoplasia, renal hypodysplasia, congenital hydronephrosis, a horseshoe kidney, posterior urethral valve and prune belly syndrome, obstructive renal dysplasia, or a nonmotile ciliopathy.

In embodiments, the abnormality has been caused by or has been correlated with a genetic factor. In embodiments, the CAKUT has been caused by or has been correlated with a genetic factor. In embodiments, the abnormality has been caused by or has been correlated with a non-genetic factor. In embodiments, the CAKUT has been caused by or has been correlated with a non-genetic factor. In embodiments, the non-genetic factor is an environmental factor.

In embodiments, the subject has a ureteral duplication, a ureteropelvic junction obstruction, renal agenesis, vesicoureteral reflux, renal hypodysplasia, congenital hydronephrosis, a horseshoe kidney, posterior urethral valve and prune belly syndrome, obstructive renal dysplasia, or a nonmotile ciliopathy. In embodiments, the subject has a ureteral duplication. In embodiments, the subject has a ureteropelvic junction obstruction. In embodiments, the subject has renal agenesis. In embodiments, the subject has vesicoureteral reflux. In embodiments, the subject has renal hypodysplasia. In embodiments, the subject has congenital hydronephrosis. In embodiments, the subject has a horseshoe kidney. In embodiments, the subject has posterior urethral valve and prune belly syndrome. In embodiments, the subject has obstructive renal dysplasia. In embodiments, the subject has a nonmotile ciliopathy. In embodiments, the CAKUT has been caused by or has been correlated with a genetic factor.

In embodiments, the anomaly comprises Alagille syndrome, Apert syndrome, Bardet-Biedl syndrome, Beckwith-Wiedemann syndrome, Branchio-Oto-Renal syndrome (BOR), Campomelic dysplasia, Cenani-Lenz syndrome, DiGeorge syndrome, Fraser syndrome, hypoparathyroidism sensorineural deafness and renal anomalies (HDR), Kallmann syndrome, Mammary-Ulnar syndrome, Meckel Gruber syndrome, nephronophthisis, Okihiro syndrome, Pallister-Hall syndrome, Renal coloboma syndrome, hypoplasia, dysplasia, renal dysplasia, cystic dysplasia, non-cystic dysplasia, VUR Cystic dysplasia, renal hypoplasia, isolated cystic renal hypoplasia, isolated non-cystic renal hypoplasia, isolated renal tubular dysgenesis, Rubinstein-Taybi syndrome, Simpson-Golabi Behmel syndrome, Townes-Brock syndrome, Zellweger syndrome, Smith-Lemli-Opitz syndrome, hydronephrosis, medullary dysplasia, unilateral/bilateral agenesis/dysplasia, collecting system anomalies, agenesis, ureteropelvic junction obstruction (UPJO) agenesis, dysplasia agenesis, unilateral agenesis, VUR, malrotation, cross-fused ectopia, VUR Dysplasia, a dual Serine/Threonine And Tyrosine Protein Kinase (DSTYK) mutation, a DSTYK mutation associated with UPJO, tubular dysgenesis, cysts, and/or aplasia.

In embodiments, the kidney disease is chronic kidney disease. In embodiments, the chronic kidney disease is Stage I, II, III, IV, or V kidney disease. In embodiments, the chronic kidney disease is Stage I kidney disease. In embodiments, the chronic kidney disease is Stage II kidney disease. In embodiments, the chronic kidney disease is Stage III kidney disease. In embodiments, the chronic kidney disease is Stage IV kidney disease. In embodiments, the chronic kidney disease is Stage V kidney disease. In embodiments, the subject is receiving dialysis at least 1, 2, or 3 times per week.

In embodiments, a population of bioactive renal cells is administered to a native organ as part of a formulation described herein. In embodiments, a secreted product of population of bioactive renal cells is administered to a native organ as part of a formulation described herein. In embodiments, the cells are sourced from the native organ that is the subject of the administration or from a source that is not the target native organ.

In embodiments, cells of the renal cell population are in the form of spheroids. In embodiments, spheroids comprising bioactive renal cells are administered to a subject. In embodiments, the spheroids comprise at least one non-renal cell type or population of cells.

In embodiments, the subject has renal disease as measured by microalbuminuria which may be defined by a urinary albumin-creatinine ratio (UACR) ≥30 mg/g or urine albumin excretion ≥30 mg/day on 24 hour urine collection.

In embodiments, the patient's kidney function is improved as a result of the treatment. An improvement of the patient's kidney function may be a stabilization of the patient's kidney function or may be a change in kidney function that improves the kidney function. In embodiments, the improved kidney function is demonstrated by a reduction in the rate of decline, stabilization of, or an increase in estimated glomerular filtration rate (eGFR). In embodiments in which the improved kidney function is demonstrated by an increase in eGFR, the increase in eGFR may be an increase of at least 1%, at least 2%, at least 3%, at least 4%, at least 5%, at least 6%, at least 7%, at least 8%, at least 9%, at least 10%, at least 15%, at least 20%, or at least 25% relative to the patient's baseline eGFR. In such embodiments, a patient's baseline eGFR may be the patient's eGFR prior to a first dose of the treatment, e.g., may be the patient's eGFR as determined at most 3 months, 2 months, 1 month, 3 weeks, 2 weeks, 10 days, 7 days, 6 days, 5 days, 4 days, 3 days, 2 days, or 1 day prior to the administration of a first dose of the treatment. The increase in the patient's baseline eGFR may be achieved within 1 to 6 months, or 2 to 6 months, or 3 to 6 months, or 4 to 6 months, or 5 to 6 months, or 1 to 5 months, or 1 to 4 months, or 1 to 3 months, or 2 to 5 months, or 2 to 4 months, or 3 to 4 months, or 2 to 3 months, or 2 months, or 3 months, or 4 months, or 5 months or 6 months following administration of a first dose of the treatment. The increase over the patient's baseline eGFR need not be at a constant level or to a constant degree, i.e., the patient need not maintain the same initial level of increase over baseline for the treatment to “improve” kidney function. The increase in patient's baseline eGFR, once achieved, may decline, provided however, that the patient's eGFR continues to be increased relative to the patient's baseline eGFR. The increase in patient's baseline eGFR, once achieved, may also be further increased or it may maintain its same level of increase over baseline as does its initial level of increase over baseline. The patient's increase in eGFR over baseline may be for over a period of time of at least 12 months, 12 months, at least 18 months, 18 months, at least 24 months, 24 months, at least 30 months, 30 months, at least 36 months, 36 months, at least 42 months, 42 months, at least 48 months, 48 months, at least 54 months, 54 months, at least 60 months, 60 months, at least 66 months, 66 months, at least 72 months, 72 months, at least 78 months, 78 months, or the remaining lifetime of the patient.

In embodiments, the improved kidney function is demonstrated by a reduction in albumin to creatinine ratio (ACR) in the patient. In embodiments in which the improved kidney function is demonstrated by a reduction in ACR in the patient, the reduction may be by at least 10%, at least 20%, at least 30%, at least 40%, at least 50%, at least 60%, at least 70%, or at least 80%, or at least 90% relative to the patient's baseline ACR. Alternatively, the reduction in ACR may be such that if the patient's baseline ACR is moderately increased, e.g., between 30 mg/g and 300 mg/g, then the reduction in ACR may reduce the patent's ACR to levels in the mild to normal range, e.g., less than 30 mg/g. Alternatively, the reduction in ACR may such that if the patient's baseline ACR is severely increased, e.g., greater than 300 mg/g, then the reduction in ACR may reduce the patient's ACR to levels that are moderately increased, e.g., between 30 mg/g and 300 mg/g, or mildly increased to normal, e.g., less than 30 mg/g. In such embodiments, a patient's baseline ACR may be the patient's ACR prior to a first dose of the treatment, e.g., may be the patient's ACR as determined at most 3 months, 2 months, 1 month, 3 weeks, 2 weeks, 10 days, 7 days, 6 days, 5 days, 4 days, 3 days, 2 days, or 1 day prior to administration of the first dose of the treatment. The reduction in the patient's baseline ACR may be achieved within 1 to 6 months, or 2 to 6 months, or 3 to 6 months, or 4 to 6 months, or 5 to 6 months, or 1 to 5 months, or 1 to 4 months, or 1 to 3 months, or 2 to 5 months, or 2 to 4 months, or 3 to 4 months, or 2 to 3 months, or 2 months, or 3 months, or 4 months, or 5 months or 6 months following administration of the first dose of the treatment. The reduction in the patient's baseline ACR need not be at a constant level or to a constant degree, i.e., the patient need not maintain the same initial level of reduction over baseline for the treatment to “improve” kidney function. The reduction in patient's baseline ACR, once achieved, may increase, provided however, that the patient's ACR continues to be reduced relative to the patient's baseline ACR. The reduction in patient's baseline ACR, once achieved, may also be further reduced or it may maintain its same level of reduction over baseline as does its initial level of reduction over baseline. The patient's reduction in ACR over baseline may be for over a period of time of at least 12 months, 12 months, at least 18 months, 18 months, at least 24 months, 24 months, at least 30 months, 30 months, at least 36 months, 36 months, at least 42 months, 42 months, at least 48 months, 48 months, at least 54 months, 54 months, at least 60 months, 60 months, at least 66 months, 66 months, at least 72 months, 72 months, at least 78 months, 78 months, or the remaining lifetime of the patient.

In embodiments, the improved kidney function is demonstrated by reduction in total serum creatinine or the rate of increase in serum creatine (sCr), or comparable measure (e.g., Cystatin-C, inulin, or other measures of glomerular filtration. In embodiments, the improved kidney function is demonstrated by improved renal cortical thickness. In embodiments, the improved kidney function may be demonstrated by structural and functional alterations. In embodiments, the improved kidney size and/or structure is determined by renal imaging. In embodiments, the method of renal imaging is ultrasound, MRI, or renal scintigraphy. In embodiments, the improved renal function is superior to the prior state of kidney structure or function.

In embodiments, the effective treatment of a kidney disease in a subject as provided herein can be observed through various indicators of kidney function. In embodiments, the indicators of kidney function include, without limitation, serum albumin level, albumin to globulin ratio (A/G ratio), serum phosphorous level, serum sodium level, kidney size (measurable by ultrasound), serum calcium level, phosphorous:calcium ratio, serum potassium level, proteinuria, urine creatinine level, serum creatinine level, blood nitrogen urea (BUN) level, cholesterol level, triglyceride levels and glomerular filtration rate (GFR).

In embodiments, several indicators of general health and well-being include, without limitation, weight gain or loss, survival, blood pressure (mean systemic blood pressure, diastolic blood pressure, or systolic blood pressure), and physical endurance performance.

In embodiments, an effective treatment with a bioactive renal cell formulation is evidenced by stabilization of one or more indicators of kidney function. In embodiments, the stabilization of kidney function is demonstrated by the observation of a change in an indicator in a subject treated by a method provided herein as compared to the same indicator in a subject that has not been treated by a method provided herein. In embodiments, the stabilization of kidney function may be demonstrated by the observation of a change in an indicator in a subject treated by a method provided herein as compared to the same indicator in the same subject prior to treatment. In embodiments, the change in the first indicator may be an increase or a decrease in value. In embodiments, the treatment provided herein may include stabilization of serum creatinine levels in a subject where the BUN levels observed in the subject are lower as compared to a subject with a similar disease state who has not been treated by the methods provided herein. In embodiments, the treatment may include stabilization of serum creatinine levels in a subject where the serum creatinine levels observed in the subject are lower as compared to a subject with a similar disease state who has not been treated by the methods provided herein. In embodiments, the stabilization of one or more of the above indicators of kidney function is the result of treatment with a selected renal cell formulation.

Those of ordinary skill in the art will appreciate that one or more additional indicators described herein or known in the art may be measured to determine the effective treatment of a kidney disease in the subject.

In embodiments, an effective treatment with a bioactive renal cell formulation is evidenced by improvement of one or more indicators of kidney function. In embodiments, the bioactive renal cell population provides an improved level of serum creatinine. In embodiments, the bioactive renal cell population provides an improved retention of protein in the serum. In embodiments, the bioactive renal cell population provides improved levels of serum cholesterol and/or triglycerides. In embodiments, the bioactive renal cell population provides an improved level of Vitamin D. In embodiments, the bioactive renal cell population provides an improved phosphorus:calcium ratio as compared to a non-enriched cell population. In embodiments, the bioactive renal cell population provides an improved level of hemoglobin as compared to a non-enriched cell population. In embodiments, the bioactive renal cell population provides an improved level of serum creatinine as compared to a non-enriched cell population. In embodiments, the improvement of one or more of the above indicators of kidney function is the result of treatment with a selected renal cell formulation.

Included herein are methods for the regeneration of a native kidney in a subject in need thereof. In embodiments, the method includes the step of administering or implanting a bioactive cell population, formulation, or construct described herein to the subject. In embodiments, a regenerated native kidney may be characterized by a number of indicators including, without limitation, development of function or capacity in the native kidney, improvement of function or capacity in the native kidney, and the expression of certain markers in the native kidney. In embodiments, the developed or improved function or capacity may be observed based on the various indicators of kidney function described above. In embodiments, the regenerated kidney is characterized by differential expression of one or more stem cell markers. In embodiments, the stem cell marker may be one or more of the following: SRY (sex determining region Y)-box 2 (Sox2); Undifferentiated Embryonic Cell Transcription Factor (UTF1); Nodal Homolog from Mouse (NODAL); Prominin 1 (PROM1) or CD133 (CD133); CD24; and any combination thereof (see Ilagan et al. PCT/US2011/036347 incorporated herein by reference in its entirety, see also Genheimer et al., 2012. Molecular characterization of the regenerative response induced by intrarenal transplantation of selected renal cells in a rodent model of chronic kidney disease. Cells Tissue Organs 196: 374-384, incorporated by reference in its entirety. In embodiments, the expression of the stem cell marker(s) is up-regulated compared to a control.

In embodiments, the effect may be provided by the cells themselves and/or by products secreted from the cells. In embodiments, a product secreted from the cells is administered to the subject. In embodiments, the product has been isolated from cells, e.g., the cells that produced it. In embodiments, the product is a vesicle as described herein. In embodiments, the vesicle (e.g., an exosome), has been isolated from the renal cell population that produced it. In embodiments, the vesicles may include one or more of the following: paracrine factors, endocrine factors, juxtacrine factors, microvesicles, exosomes, and RNA. The secreted products may also include products that are not within microvesicles including, without limitation, paracrine factors, endocrine factors, juxtacrine factors, and RNA. In embodiments, the secreted products may be part of a vesicle derived from renal cells. In embodiments, the vesicles are secreted vesicles. In embodiments, the secreted vesicles are exosomes, microvesicles, ectosomes, membrane particles, exosome-like vesicles, or apoptotic vesicles. In embodiments, the secreted vesicles are exosomes. In embodiments, the secreted vesicles are microvesicles. In embodiments, the secreted vesicles contain or comprise one or more cellular components. In embodiments, the components may be one or more of the following: membrane lipids, RNA, proteins, metabolities, cytosolic components, and any combination thereof. In embodiments, the secreted vesicles comprise one or more microRNAs. In embodiments, the one or more miRNAs include one of or any combination of RNA (e.g., miRNA) molecules disclosed herein. In embodiments, the vesicles comprise an miRNA that inhibits Plasminogen Activation Inhibitor-1 (PAI-1) and/or TGFβ1. In embodiments, the secreted product that comprises a paracrine and/or juxtacrine factor, such as alpha-1 microglobulin, beta-2-microglobulin, calbindin, clusterin, connective tissue growth factor, cystatin-C, glutathione-S-transferase alpha, kidney injury moleculte-1, neutraphil gelatinase-associated lipocalin, osteopontin, trefoil factor 3, tam-horsfall urinary glycoprotein, tissue-inhibitor of metallo proteinase 1, vascular endothelial growth factor, fibronectin, interleukin-6, or monocyte chemotactic protein-1.

In embodiments, the effect may be provided by the cells themselves and/or by products secreted from the cells. In embodiments, regenerative effect may be characterized by one or more of the following: a reduction in epithelial-mesenchymal transition (which may be via attenuation of TGF-β signaling); a reduction in renal fibrosis; a reduction in renal inflammation; differential expression of a stem cell marker in the native kidney; migration of implanted cells and/or native cells to a site of renal injury, e.g., tubular injury, engraftment of implanted cells at a site of renal injury, e.g., tubular injury; stabilization of one or more indicators of kidney function (as described herein); de novo formation of S-shaped bodies/comma-shaped bodies associated with nephrogenesis, de novo formation of renal tubules or nephrons, restoration of erythroid homeostasis (as described herein); and any combination thereof. (see also Basu et al., 2011. Functional evaluation of primary renal cell/biomaterial neo-kidney augment prototypes for renal tissue engineering. Cell Transplantation 20: 1771-90; Bruce et al., 2015. Selected renal cells modulate disease progression in rodent models of chronic kidney disease via NF-κB and TGF-β1 pathways. Regenerative Medicine 10: 815-839, the entire content of each of which is incorporated herein by reference).

In embodiments, in addition to a tissue biopsy or as an alternative to a tissue biopsy, a regenerative outcome in a subject receiving treatment can be assessed from examination of a bodily fluid, e.g., urine. It has been discovered that microvesicles obtained from subject-derived urine sources contain certain components including, without limitation, specific proteins and miRNAs that are ultimately derived from the renal cell populations. In embodiments, these components may include factors involved in stem cell replication and differentiation, apoptosis, inflammation and immuno-modulation. In embodiments, a temporal analysis of microvesicle-associated miRNA/protein expression patterns allows for continuous monitoring of regenerative outcomes within the kidney of subjects receiving the cell populations or constructs described herein.

Also provided are methods of assessing whether a kidney disease patient is responsive to treatment with a therapeutic formulation. In embodiments, the method may include the step of determining or detecting the amount of vesicles or a luminal content or contents thereof in a test sample obtained from a kidney disease patient treated with the therapeutic, as compared to or relative to the amount of vesicles in a control sample, wherein a higher or lower amount of vesicles or one or more luminal contents thereof in the test sample as compared to the amount of vesicles or luminal content(s) in the control sample is indicative of the treated patient's responsiveness to treatment with the therapeutic.

In embodiments, these kidney-derived vesicles and/or the luminal contents of kidney derived vesicles may also be shed into the urine of a subject and may be analyzed for biomarkers indicative of regenerative outcome or treatment efficacy. In embodiments, the non-invasive prognostic methods may include the step of obtaining a urine sample from the subject before and/or after administration or implantation of a cell population, composition, formulation, or construct described herein. Vesicles and other secreted products may be isolated from the urine samples using standard techniques including without limitation, centrifugation to remove unwanted debris (Zhou et al. 2008. Kidney Int. 74(5):613-621; Skog et al. U.S. Published Patent Application No. 20110053157, each of which is incorporated herein by reference in its entirety).

In embodiments, the vesicles may include one or more of the following: paracrine factors, endocrine factors, juxtacrine factors, microvesicles, exosomes, and RNA. The secreted products may also include products that are not within microvesicles including, without limitation, paracrine factors, endocrine factors, juxtacrine factors, and RNA.

In embodiments, the secreted products may be part of a vesicle derived from renal cells. In embodiments, the vesicles are secreted vesicles. In embodiments, the secreted vesicles are exosomes, microvesicles, ectosomes, membrane particles, exosome-like vesicles, or apoptotic vesicles. In embodiments, the secreted vesicles are exosomes. In embodiments, the secreted vesicles are microvesicles. In embodiments, the secreted vesicles contain or comprise one or more cellular components. In embodiments, the components may be one or more of the following: membrane lipids, RNA, proteins, metabolities, cytosolic components, and any combination thereof. In embodiments, the secreted vesicles comprise one or more microRNAs. In embodiments, the one or more miRNAs include one of or any combination of miR-30b-5p, miR-449a, miR-146a, miR-130a, miR-23b, miR-21, miR-124, and miR-151. In embodiments, the one or more miRNAs include one of or any combination of let-7a-1; let-7a-2; let-7a-3; let-7b; let-7c; let-7d; let-7e; let-7f-1; let-7f-2; let-7g; let-7i; mir-1-1; mir-1-2; mir-7-1; mir-7-2; mir-7-3; mir-9-1; mir-9-2; mir-9-3; mir-10a; mir-10b; mir-15a; mir-15b; mir-16-1; mir-16-2; mir-17; mir-18a; mir-18b; mir-19a; mir-19b-1; mir-19b-2; mir-20a; mir-20b; mir-21; mir-22; mir-23a; mir-23b; mir-23c; mir-24-1; mir-24-2; mir-25; mir-26a-1; mir-26a-2; mir-26b; mir-27a; mir-27b; mir-28; mir-29a; mir-29b-1; mir-29b-2; mir-29c; mir-30a; mir-30b; mir-30c-1; mir-30c-2; mir-30d; mir-30e; mir-31; mir-32; mir-33a; mir-33b; mir-34a; mir-34b; mir-34c; mir-92a-1; mir-92a-2; mir-92b; mir-93; mir-95; mir-96; mir-98; mir-99a mir-99b; mir-100; mir-101-1; mir-101-2; mir-103-1; mir-103-1-as; mir-103-2; mir-103-2-as; mir-105-1; mir-105-2; mir-106a; mir-106b; mir-107; mir-122; mir-124-1; mir-124-2; mir-124-3; mir-125a; mir-125b-1; mir-125b-2; mir-126; mir-127; mir-128-1; mir-128-2; mir-129-1; mir-129-2; mir-130a; mir-130b; mir-132; mir-132; mir-133a-1; mir-133a-2; mir-133b; mir-134; mir-135a-1; mir-135a-2; mir-135b; mir-136 MI101351120; mir-137; mir-138-1; mir-138-2; mir-139; mir-140; mir-141; mir-142; mir-143; mir-144; mir-145; mir-146a; mir-146b; mir-147; mir-147b; mir-148a; mir-148b; mir-149; mir-150; mir-151; mir-152; mir-153-1; mir-153-2; mir-154; mir-155; mir-181a-1; mir-181a-2; mir-181b-1; mir-181b-2; mir-181c; mir-181d; mir-182; mir-183; mir-184; mir-185; mir-186; mir-187; mir-188; mir-190; mir-190b; mir-191; mir-192; mir-193a; mir-193b; mir-194-1; mir-194-2; mir-195; mir-196a-1; mir-196a-2; mir-196b; mir-197; mir-198; mir-199a-1; mir-199a-2; mir-199b; mir-200a; mir-200b; mir-200c; mir-202; mir-203; mir-204; mir-205; mir-206; mir-208a; mir-208b; mir-210; mir-211; mir-212; mir-214; mir-215; mir-216a; mir-216b; mir-217; mir-218-1; mir-218-2; mir-219-1; mir-219-2; mir-221; mir-222; mir-223; mir-224; mir-296; mir-297; mir-298; mir-299; mir-300; mir-301a; mir-301b; mir-302a; mir-302b; mir-302c; mir-302d; mir-302e; mir-302f; mir-320a; mir-320b-1; mir-320b-2; mir-320c-1; mir-320c-2; mir-320d-1; mir-320d-2; mir-320e; mir-323; mir-323b; mir-324; mir-325; mir-326; mir-328; mir-329-1; mir-329-2; mir-330; mir-331; mir-335; mir-337; mir-338; mir-339; mir-340; mir-342; mir-345; mir-346; mir-361; mir-362; mir-363; mir-365-1; mir-365-2; mir-367; mir-369; mir-370; mir-37; mir-372; mir-373; mir-374a; mir-374b; mir-374c; mir-375; mir-376a-1; mir-376a-2; mir-376b; mir-376c; mir-377; mir-378; mir-378b; mir-378c; mir-379; mir-380; mir-381; mir-382; mir-383; mir-384; mir-409; mir-410; mir-411; mir-412; mir-421; mir-422a; mir-423; mir-424; mir-425; mir-429; mir-431; mir-432; mir-433; mir-448; mir-449a; mir-449b; mir-449c; mir-450a-1; mir-450a-2; mir-450b; mir-451; mir-452; mir-454; mir-455; mir-466; mir-483; mir-484; mir-485; mir-486; mir-487a; mir-487b; mir-488; mir-489; mir-490; mir-491; mir-492; mir-493; mir-494; mir-495; mir-496; mir-497; mir-498; mir-499; mir-500a; mir-500b; mir-501; mir-502; mir-503; mir-504; mir-505; mir-506; mir-507; mir-508; mir-509-1; mir-509-2; mir-509-3; mir-510; mir-511-1; mir-511-2; mir-512-1; mir-512-2; mir-513a-1; mir-513a-2; mir-513b; mir-513c; mir-514-1; mir-514-2; mir-514-3; mir-514b; mir-515-1; mir-515-2; mir-516a-1; mir-516a-2; mir-516b-1; mir-516b-2; mir-517a; mir-517b; mir-517c; mir-518a-1; mir-518a-2; mir-518b; mir-518c; mir-518d; mir-518e; mir-518f; mir-519a-1; mir-519a-2; mir-519b; mir-519c; mir-519d; mir-519e; mir-520a; mir-520b; mir-520c; mir-520d; mir-520e; mir-520f; mir-520g; mir-520h; mir-521-1; mir-521-2; mir-522; mir-523; mir-524; mir-525; mir-526a-1; mir-526a-2; mir-526b; mir-527; mir-532; mir-539; mir-541; mir-542; mir-543; mir-544; mir-544b; mir-545; mir-548a-1; mir-548a-2; mir-548a-3; mir-548ah-1; mir-548ah-2; mir-548b; mir-548c; mir-548d-1; mir-548d-2; mir-548e; mir-548f-1; mir-548f-2; mir-548f-3; mir-548f-4; mir-548f-5; mir-548g; mir-548h-1; mir-548h-2; mir-548h-3; mir-548h-4; mir-548i-1; mir-548i-2; mir-548i-3; mir-548i-4; mir-548j; mir-548k; mir-548l; mir-548m; mir-548n; mir-548o; mir-548p; mir-548s; mir-548t; mir-548u; mir-548v; mir-548w; mir-548x; mir-548y; mir-548z; mir-549; mir-550a-1; mir-550a-2; mir-550b-1; mir-550b-2; mir-551a; mir-551b; mir-552; mir-553; mir-554; mir-555; mir-556; mir-557; mir-558; mir-559; mir-561; mir-562; mir-563; mir-564; mir-566; mir-567; mir-568; mir-569; mir-570; mir-571; mir-572; mir-573; mir-574; mir-575; mir-576; mir-577; mir-578; mir-579; mir-580; mir-581; mir-582; mir-583; mir-584; mir-585; mir-586; mir-587; mir-588; mir-589; mir-590; mir-591; mir-592; mir-593; mir-595; mir-596; mir-597; mir-598; mir-599; mir-600; mir-601; mir-602; mir-603; mir-604; mir-605; mir-606; mir-607; mir-608; mir-609; mir-610; mir-611; mir-612; mir-613; mir-614; mir-615; mir-616; mir-617; mir-618; mir-619; mir-620; mir-621; mir-622; mir-623; mir-624; mir-625; mir-626; mir-627; mir-628; mir-629; mir-630; mir-631; mir-632; mir-633; mir-634; mir-635; mir-636; mir-637; mir-638; mir-639; mir-640; mir-641; mir-642a; mir-642b; mir-643; mir-644; mir-645; mir-646; mir-647; mir-648; mir-649; mir-650; mir-651; mir-652; mir-653; mir-654; mir-655; mir-656; mir-657; mir-658; mir-659; mir-660; mir-661; mir-662; mir-663; mir-663b; mir-664; mir-665; mir-668; mir-670; mir-671; mir-675; mir-676; mir-708; mir-711; mir-718; mir-720; mir-744; mir-758; mir-759; mir-760; mir-761; mir-762; mir-764; mir-765; mir-766; mir-767; mir-769; mir-770; mir-802; mir-873; mir-874; mir-875; mir-876; mir-877; mir-885; mir-887; mir-888; mir-889; mir-890; mir-891a; mir-891b; mir-892a; mir-892b; mir-920; mir-921; mir-922; mir-924; mir-933; mir-934; mir-935; mir-936; mir-937; mir-938; mir-939; mir-940; mir-941-1; mir-941-2; mir-941-3; mir-941-4; mir-942; mir-942; mir-943; mir-944; mir-1178; mir-1179; mir-1180; mir-1181; mir-1182; mir-1183; mir-1184-1; mir-1184-2; mir-1184-3; mir-1185-1; mir-1185-2; mir-1193; mir-1197; mir-1200; mir-1202; mir-1203; mir-1204; mir-1205; mir-1206; mir-1207; mir-1208; mir-1224; mir-1225; mir-1226; mir-1227; mir-1228; mir-1229; mir-1231; mir-1233-1; mir-1233-2; mir-1234; mir-1236; mir-1237; mir-1238; mir-1243; mir-1244-1; mir-1244-2; mir-1244-3; mir-1245; mir-1246; mir-1247; mir-1248; mir-1249; mir-1250; mir-1251; mir-1252; mir-1253; mir-1254; mir-1255a; mir-1255b-1; mir-1255b-2; mir-1256; mir-1257; mir-1258; mir-1260; mir-1260b; mir-1261; mir-1262; mir-1263; mir-1264; mir-1265; mir-1266; mir-1267; mir-1268; mir-1269; mir-1270-1; mir-1270-2; mir-1271; mir-1272; mir-1273; mir-1273c; mir-1273d; mir-1273e; mir-1274a; mir-1274b; mir-1275; mir-1276; mir-1277; mir-1278; mir-1279; mir-1280; mir-1281; mir-1282; mir-1283-1; mir-1283-2; mir-1284; mir-1285-1; mir-1285-2; mir-1286; mir-1287; mir-1288; mir-1289-1; mir-1289-2; mir-1290; mir-1291; mir-1292; mir-1293; mir-1294; mir-1295; mir-1296; mir-1297; mir-1298; mir-1299; mir-1301; mir-1302-1; mir-1302-10; mir-1302-11; mir-1302-2; mir-1302-3; mir-1302-4; mir-1302-5; mir-1302-6; mir-1302-7; mir-1302-8; mir-1302-9; mir-1303; mir-1304; mir-1305; mir-1306; mir-1307; mir-1321; mir-1322; mir-1323; mir-1324; mir-1468; mir-1469; mir-1470; mir-1471; mir-1537; mir-1538; mir-1539; mir-1825; mir-1827; mir-1908; mir-1909; mir-1910; mir-1911; mir-1912; mir-1913; mir-1914; mir-1915; mir-1972-1; mir-1972-2; mir-1973; mir-1976; mir-2052; mir-2053; mir-2054; mir-2110; mir-2113; mir-2114; mir-2115; mir-2116; mir-2117; mir-2276; mir-2277; mir-2278; mir-2355; mir-2861; mir-2909; mir-3065; mir-3074; mir-3115; mir-3116-1; mir-3116-2; mir-3117; mir-3118-1; mir-3118-2; mir-3118-3; mir-3118-4; mir-3118-5; mir-3118-6; mir-3119-1; mir-3119-2; mir-3120; mir-3121; mir-3122; mir-3123; mir-3124; mir-3125; mir-3126; mir-3127; mir-3128; mir-3129; mir-3130-1; mir-3130-2; mir-3131; mir-3132; mir-3133; mir-3134; mir-3135; mir-3136; mir-3137; mir-3138; mir-3139; mir-3140; mir-3141; mir-3142; mir-3143; mir-3144; mir-3145; mir-3146; mir-3147; mir-3148; mir-3149; mir-3150; mir-3151; mir-3152; mir-3153; mir-3154; mir-3155; mir-3156-1; mir-3156-2; mir-3156-3; mir-3157; mir-3158-1; mir-3158-2; mir-3159; mir-3160-1; mir-3160-2; mir-3161; mir-3162; mir-3163; mir-3164; mir-3165; mir-3166; mir-3167; mir-3168; mir-3169; mir-3170; mir-3171; mir-3173; mir-3174; mir-3175; mir-3176; mir-3177; mir-3178; mir-3179-1; mir-3179-2; mir-3179-3; mir-3180-1; mir-3180-2; mir-3180-3; mir-3180-4; mir-3180-5; mir-3181; mir-3182; mir-3183; mir-3184; mir-3185; mir-3186; mir-3187; mir-3188; mir-3189; mir-3190; mir-3191; mir-3192; mir-3193; mir-3194; mir-3195; mir-3196; mir-3197; mir-3198; mir-3199-1; mir-3199-2; mir-3200; mir-3201; mir-3202-1; mir-3202-2; mir-3605; mir-3606; mir-3607; mir-3609; mir-3610; mir-3611; mir-3612; mir-3613; mir-3614; mir-3615; mir-3616; mir-3617; mir-3618; mir-3619; mir-3620; mir-3621; mir-3622a; mir-3622b; mir-3646; mir-3647; mir-3648; mir-3649; mir-3650; mir-3651; mir-3652; mir-3653; mir-3654; mir-3655; mir-3656mir-3657; mir-3658; mir-3659; mir-3660; mir-3661; mir-3662; mir-3663; mir-3664; mir-3665; mir-3666; mir-3667; mir-3668; mir-3669; mir-3670; mir-3670; mir-3671; mir-3671; mir-3673; mir-3673; mir-3675; mir-3675; mir-3676; mir-3663; mir-3677; mir-3678; mir-3679; mir-3680; mir-3681; mir-3682; mir-3683; mir-3684; mir-3685; mir-3686; mir-3687; mir-3688; mir-3689a; mir-3689b; mir-3690; mir-3691; mir-3692; mir-3713; mir-3714; mir-3907; mir-3908; mir-3909; mir-3910-1; mir-3910-2; mir-3911; mir-3912; mir-3913-1; mir-3913-2; mir-3914-1; mir-3914-2; mir-3915; mir-3916; mir-3917; mir-3918; mir-3919; mir-3920; mir-3921; mir-3922; mir-3923; mir-3924; mir-3925; mir-3926-1; mir-3926-2; mir-3927; mir-3928; mir-3929; mir-3934; mir-3935; mir-3936; mir-3937; mir-3938; mir-3939; mir-3940; mir-3941; mir-3942; mir-3943; mir-3944; mir-3945; mir-4251; mir-4252; mir-4253; mir-4254; mir-4255; mir-4256; mir-4257; mir-4258; mir-4259; mir-4260; mir-4261; mir-4262; mir-4263; mir-4264; mir-4265; mir-4266; mir-4267; mir-4268; mir-4269; mir-4270; mir-4271; mir-4272; mir-4273; mir-4274; mir-4275; mir-4276; mir-4277; mir-4278; mir-4279; mir-4280; mir-4281; mir-4282; mir-4283-1; mir-4283-2; mir-4284; mir-4285; mir-4286; mir-4287; mir-4288; mir-4289; mir-4290; mir-4291; mir-4292; mir-4293; mir-4294; mir-4295; mir-4296; mir-4297; mir-4298; mir-4299; mir-4300; mir-4301; mir-4302; mir-4303; mir-4304; mir-4305; mir-4306; mir-4307; mir-4308; mir-4309; mir-4310; mir-4311; mir-4312; mir-4313; mir-4314; mir-4315-1; mir-4315-2; mir-4316; mir-4317; mir-4318; mir-4319; mir-4320; mir-4321; mir-4322; mir-4323; mir-4324; mir-4325; mir-4326; mir-4327; mir-4328; mir-4329; mir-4329; and mir-4330.

In embodiments, the miRNAs include any one of, or two or more of, the following: miR-21; miR-23a; miR-30c; miR-1224; miR-23b; miR-92a; miR-100; miR-125b-5p; miR-195; miR-10a-5p; and any combination thereof. In embodiments, the miRNAs include any one of, or two or more of, the following: miR-30b-5p, miR-449a, miR-146a, miR-130a, miR-23b, miR-21, miR-124, miR-151, and any combination thereof. In embodiments, the miRNAs include any one of, or two or more of, the following: miR-24, miR-195, miR-871, miR-30b-5p, miR-19b, miR-99a, miR-429, let-7f, miR-200a, miR-324-5p, miR-10a-5p, and any combination thereof. In embodiments, the combination of miRNAs may include 2, 3, 4, 5, 6, 7, 8, 9, 10, or more individual miRNAs.

In embodiments the secreted product comprises a compound that attenuated a NFkB signaling pathway.

In embodiments, the secreted product comprises a paracrine factor. In embodiments, paracrine factors are molecules synthesized by a cell that can diffuse over small distances to induce or effect changes in a neighboring cell, i.e., a paracrine interaction. In embodiments, the diffusible molecules are referred to as paracrine factors. In embodiments, juxtacrine factors are molecules that facilitate intercellular communication that is transmitted via oligosaccharide, lipid, or protein components of a cell membrane, and may affect either the emitting cell or the immediately adjacent cells. In embodiments, juxtacrine signaling typically involves physical contact between the two cells involved.

In embodiments, the vesicles comprise an miRNA that inhibits Plasminogen Activation Inhibitor-1 (PAI-1) and/or TGFβ1.

In embodiments, the secreted product that comprises a paracrine and/or juxtacrine factor, such as alpha-1 microglobulin, beta-2-microglobulin, calbindin, clusterin, connective tissue growth factor, cystatin-C, glutathione-S-transferase alpha, kidney injury moleculte-1, neutraphil gelatinase-associated lipocalin, osteopontin, trefoil factor 3, tam-horsfall urinary glycoprotein, tissue-inhibitor of metallo proteinase 1, vascular endothelial growth factor, fibronectin, interleukin-6, or monocyte chemotactic protein-1.

In embodiments, the effective treatment of a kidney disease in a subject by the methods disclosed herein can be observed through various indicators of erythropoiesis and/or kidney function. In embodiments, the indicators of erythroid homeostasis include, without limitation, hematocrit (HCT), hemoglobin (HB), mean corpuscular hemoglobin (MCH), red blood cell count (RBC), reticulocyte number, reticulocyte %, mean corpuscular volume (MCV), and red blood cell distribution width (RDW). In embodiments, the indicators of kidney function include, without limitation, serum albumin, albumin to globulin ratio (A/G ratio), serum phosphorous, serum sodium, kidney size (measurable by ultrasound), serum calcium, phosphorous:calcium ratio, serum potassium, proteinuria, urine creatinine, serum creatinine, blood nitrogen urea (BUN), cholesterol levels, triglyceride levels and glomerular filtration rate (GFR). Furthermore, several indicators of general health and well-being include, without limitation, weight gain or loss, survival, blood pressure (mean systemic blood pressure, diastolic blood pressure, or systolic blood pressure), and physical endurance performance.

In embodiments, an effective treatment with a bioactive renal cell formulation is evidenced by stabilization of one or more indicators of kidney function. In embodiments, the stabilization of kidney function is demonstrated by the observation of a change in an indicator in a subject treated by a method provided for herein as compared to the same indicator in a subject that has not been treated by the method herein. In embodiments, the stabilization of kidney function may be demonstrated by the observation of a change in an indicator in a subject treated by a method herein as compared to the same indicator in the same subject prior to treatment. In embodiments, the change in the first indicator may be an increase or a decrease in value. In embodiments, the treatment provided by the present disclosure may include stabilization of blood urea nitrogen (BUN) levels in a subject where the BUN levels observed in the subject are lower as compared to a subject with a similar disease state who has not been treated by the methods of the present disclosure. In embodiments, the treatment may include stabilization of serum creatinine levels in a subject where the serum creatinine levels observed in the subject are lower as compared to a subject with a similar disease state who has not been treated by the methods of the present disclosure. In embodiments, the treatment may include stabilization of hematocrit (HCT) levels in a subject where the HCT levels observed in the subject are higher as compared to a subject with a similar disease state who has not been treated by the methods of the present disclosure. In embodiments, the treatment may include stabilization of red blood cell (RBC) levels in a subject where the RBC levels observed in the subject are higher as compared to a subject with a similar disease state who has not been treated by the methods of the present disclosure. In embodiments, one or more additional indicators described herein or known in the art may be measured to determine the effective treatment of a kidney disease in the subject.

In embodiments, a regenerated native kidney may be characterized by a number of indicators including, without limitation, development of function or capacity in the native kidney, improvement of function or capacity in the native kidney, and the expression of certain markers in the native kidney. In embodiments, the developed or improved function or capacity may be observed based on the various indicators of erythroid homeostasis and kidney function described herein. In embodiments, the regenerated kidney is characterized by differential expression of one or more stem cell markers. In embodiments, the stem cell marker may be one or more of the following: Sox2; UTF1; NODAL; PROM1 or CD133; CD24; and any combination thereof (see Ilagan et al. PCT/US2011/036347 incorporated herein by reference in its entirety). In embodiments, the expression of the stem cell marker(s) is up-regulated compared to a control.

In embodiments, the cell populations described herein, including enriched cell populations and/or admixtures thereof, as well as constructs containing the same may be used to provide a regenerative effect to a native kidney. In embodiments, the effect may be provided by the cells themselves and/or by products secreted from the cells. In embodiments, the regenerative effect may be characterized by one or more of the following: a reduction in epithelial-mesenchymal transition (which may be via attenuation of TGF-β signaling); a reduction in renal fibrosis; a reduction in renal inflammation; differential expression of a stem cell marker in the native kidney; migration of implanted cells and/or native cells to a site of renal injury, e.g., tubular injury, engraftment of implanted cells at a site of renal injury, e.g., tubular injury; stabilization of one or more indicators of kidney function (as described herein); restoration of erythroid homeostasis (as described herein); and any combination thereof.

In embodiments, a therapeutic composition or formulation provided herein contains an isolated, heterogeneous population of kidney cells that is enriched for specific bioactive components or cell types and/or depleted of specific inactive or undesired components or cell types. In embodiments, such compositions and formulations are used in the treatment of kidney disease, e.g., providing stabilization and/or improvement and/or regeneration of kidney function and/or structure. In embodiments, the compositions contain isolated renal cell fractions that lack cellular components as compared to a healthy individual yet retain therapeutic properties, e.g., provide stabilization and/or improvement and/or regeneration of kidney function. In embodiments, the cell populations described herein may be derived from healthy individuals, individuals with a kidney disease, or subjects as described herein.

Included herein are therapeutic compositions of selected renal cell populations that are to be administered to a target organ or tissue in a subject. In embodiments, a bioactive selected renal cell population generally refers to a cell population potentially having therapeutic properties upon administration to a subject. In embodiments, upon administration to a subject in need, a bioactive renal cell population can provide stabilization and/or improvement and/or repair and/or regeneration of kidney function in the subject. In embodiments, the therapeutic properties may include a repair or regenerative effect.

In embodiments, the renal cell population is an unfractionated, heterogeneous cell population or an enriched homogeneous cell population derived from a kidney. In embodiments, the heterogeneous cell population is isolated from a tissue biopsy or from whole organ tissue. In embodiments, the renal cell population is derived from an in vitro culture of mammalian cells, established from tissue biopsies or whole organ tissue. In embodiments, a renal cell population comprises subfractions or subpopulations of a heterogeneous population of renal cells, enriched for bioactive components (e.g., bioactive renal cells) and depleted of inactive or undesired components or cells.

In embodiments, the renal cell population expresses GGT and a cytokeratin. In embodiments, the GGT has a level of expression greater than about 10%, about 15%, about 18%, about 20%, about 25%, about 30%, about 35%, about 40%, about 45%, about 50%, about 55%, or about 60%. In embodiments, the GGT is GGT-1. In embodiments, cells of the renal cell population expresses GGT-1, a cytokeratin, VEGF, and KIM-1. In embodiments, greater than 18% of the cells in the renal cell population express GGT-1. In embodiments, greater than 80% of the cells in the renal cell population express the cytokeratin. In embodiments, the cytokeratin is selected from CK8, CK18, CK19 and combinations thereof. In embodiments, the cytokeratin is CK8, CK18, CK19, CK8/CK18, CK8/CK19, CK18/CK19 or CK8/CK18/CK19, wherein the “/” refers to a combination of the cytokeratins adjacent thereto. In embodiments, the cytokeratin has a level of expression greater than about 80%, about 85%, about 90%, or about 95%. In embodiments, greater than 80% of the cells in the renal cell population express the cytokeratin. In embodiments, the renal cell population expresses AQP2. In embodiments, less than 40% of the cells express AQP2. In embodiments, at least 3% of the cells in the renal cell population express AQP2.

In embodiments, greater than 18% of the cells within the cell population express GGT-1 and greater than 80% of the cells within the cell population express a cytokeratin. In embodiments, the cytokeratin is CK18. In embodiments, 4.5% to 81.2% of the cells in the cell population express GGT-1, 3.0% to 53.7% of the cells within the cell population express AQP2, and 81.1% to 99.7% of the cells within the cell population express CK18.

In embodiments, the renal cell population comprises cells that express one or more of any combination of the biomarkers selected from AQP1, AQP2, AQP4, Calbindin, Calponin, CD117, CD133, CD146, CD24, CD31 (PECAM-1), CD54 (ICAM-1), CD73, CK18, CK19, CK7, CK8, CK8, CK18, CK19, combinations of CK8, CK18 and CK19, Connexin 43, Cubilin, CXCR4 (Fusin), DBA, E-cadherin (CD324), EPO (erythropoeitin) GGT1, GLEPP1 (glomerular epithelial protein 1), Haptoglobulin, Itgb1 (Integrin 01), KIM-1 (kidney injury molecule-1), TIM-1 (T-cell immunoglobulin and mucirs-containing molecule), MAP-2(microtubule-associated protein 2), Megalin, N-cadherin, Nephrin, NKCC (Na-K-Cl-cotransporters), OAT-1 (organic anion transporter 1), Osteopontin, Pan-cadherin, PCLP1 (podocalyxin-like 1 molecule), Podocin, SMA (smooth muscle alpha-actin), Synaptopodin, THP (tamm-horsfall protein), Vinientin, and αGST-1 (alpha glutathione S-transferase).

In embodiments, the renal cell population is enriched for epithelial cells compared to a starting population, such as a population of cells in a kidney tissue biopsy or a primary culture thereof (e.g., the renal cell population comprises at least about 5%, 10%, 15%, 20%, or 25% more epithelial cells than the starting population). In embodiments, the renal cell population is enriched for tubular cells compared to a starting population, such as a population of cells in a kidney tissue biopsy or a primary culture thereof (e.g., the renal cell population comprises at least about 5%, 10%, 15%, 20%, or 25% more tubular cells than the starting population). In embodiments, the tubular cells comprise proximal tubular cells. In embodiments, the renal cell population has a lesser proportion of distal tubular cells, collecting duct cells, endocrine cells, vascular cells, or progenitor-like cells compared to the starting population. In embodiments, the renal cell population has a lesser proportion of distal tubular cells compared to the starting population. In embodiments, the renal cell population has a lesser proportion of collecting duct cells compared to the starting population. In embodiments, the renal cell population has a lesser proportion of endocrine cells compared to the starting population. In embodiments, the renal cell population has a lesser proportion of vascular cells compared to the starting population. In embodiments, the renal cell population has a lesser proportion of progenitor-like cells compared to the starting population. In embodiments, the renal cell population has a greater proportion of tubular cells and lesser proportions of EPO producing cells, glomerular cells, and vascular cells when compared to the non-enriched population (e.g., a starting kidney cell population). In embodiments, the renal cell population has a greater proportion of tubular cells and lesser proportions of EPO producing cells and vascular cells when compared to the non-enriched population. In embodiments, the renal cell population has a greater proportion of tubular cells and lesser proportions of glomerular cells and vascular cells when compared to the non-enriched population.

In embodiments, cells of the renal cell population, express hyaluronic acid (HA). In embodiments, the size range of HA is from about 5 kDa to about 20000 kDa. In embodiments, the HA has a molecular weight of 5 kDa, 60 kDa, 800 kDa, and/or 3,000 kDa. In embodiments, the renal cell population synthesizes and/or stimulate synthesis of high molecular weight HA through expression of Hyaluronic Acid Synthase-2 (HAS-2), especially after intra-renal implantation. In embodiments, cells of the renal cell population express higher molecular weight species of HA in vitro and/or in vivo, through the actions of HAS-2. In embodiments, cells of the renal cell population express higher molecular weight species of HA both in vitro and in vivo, through the actions of HAS-2. In embodiments, a higher molecular weight species of HA is HA having a molecular weight of at least 100 kDa. In embodiments, the higher molecular weight species of HA is HA having a molecular weight from about 800 kDa to about 3,500 kDa. In embodiments, the higher molecular weight species of HA is HA having a molecular weight from about 800 kDa to about 3,000 kDa. In embodiments, the higher molecular weight species of HA is HA having a molecular weight of at least 800 kDa. In embodiments, the higher molecular weight species of HA is HA having a molecular weight of at least 3,000 kDa. In embodiments, the higher molecular weight species of HA is HA having a molecular weight of about 800 kDa. In embodiments, the higher molecular weight species of HA is HA having a molecular weight of about 3,000 kDa. In embodiments, HAS-2 synthesizes HA with a molecular weight of 2×10⁵ to 2×10⁶ Da.

In embodiments, smaller species of HA are formed through the action of degradative hyaluronidases. In embodiments, the higher molecular weight species of HA is HA having a molecular weight from about 200 kDa to about 2000 kDa. In embodiments, the higher molecular weight species of HA is HA having a molecular weight of about 200 kDa. In embodiments, the higher molecular weight species of HA is HA having a molecular weight of about 2000 kDa. In embodiments, the higher molecular weight species of HA is HA having a molecular weight of at least 200 kDa. In embodiments, the higher molecular weight species of HA is HA having a molecular weight of at least 2000 kDa. In embodiments, the higher molecular weight species of HA is HA having a molecular weight of at least 5000 kDa. In embodiments, the higher molecular weight species of HA is HA having a molecular weight of at least 10000 kDa. In embodiments, the higher molecular weight species of HA is HA having a molecular weight of at least 15000 kDa. In embodiments, the higher molecular weight species of HA is HA having a molecular weight of about 20000 kDa.

In embodiments, the population comprises cells that are capable of receptor-mediated albumin transport.

In embodiments, cells of the renal cell population are hypoxia resistant.

In embodiments, the renal cell population comprises one or more cell types that express one or more of any combination of: megalin, cubilin, N-cadherin, E-cadherin, Aquaporin-1, and Aquaporin-2.

In embodiments, the renal cell population comprises one or more cell types that express one or more of any combination of: megalin, cubilin, hyaluronic acid synthase 2 (HAS2), Vitamin D3 25-Hydroxylase (CYP2D25), N-cadherin (Ncad), E-cadherin (Ecad), Aquaporin-1 (Aqp1), Aquaporin-2 (Aqp2), RAB17, member RAS oncogene family (Rab17), GATA binding protein 3 (Gata3), FXYD domain-containing ion transport regulator 4 (Fxyd4), solute carrier family 9 (sodium/hydrogen exchanger), member 4 (Slc9a4), aldehyde dehydrogenase 3 family, member B1 (Aldh3b1), aldehyde dehydrogenase 1 family, member A3 (Aldh1a3), and Calpain-8 (Capn8).

In embodiments, the renal cell population comprises one or more cell types that express one or more of any combination of: megalin, cubilin, hyaluronic acid synthase 2 (HAS2), Vitamin D3 25-Hydroxylase (CYP2D25), N-cadherin (Ncad), E-cadherin (Ecad), Aquaporin-1 (Aqp1), Aquaporin-2 (Aqp2), RAB17, member RAS oncogene family (Rab17), GATA binding protein 3 (Gata3), FXYD domain-containing ion transport regulator 4 (Fxyd4), solute carrier family 9 (sodium/hydrogen exchanger), member 4 (Slc9a4), aldehyde dehydrogenase 3 family, member 81 (Aldh3b1), aldehyde dehydrogenase 1 family, member A3 (Aldh1a3), and Calpain-8 (Capn8), and Aquaporin-4 (Aqp4).

In embodiments, the renal cell population comprises one or more cell types that express one or more of any combination of: aquaporin 7 (Aqp7), FXYD domain-containing ion transport regulator 2 (Fxyd2), solute carrier family 17 (sodium phosphate), member 3 (Slc17a3), solute carrier family 3, member 1 (Slc3al), claudin 2 (Cldn2), napsin A aspartic peptidase (Napsa), solute carrier family 2 (facilitated glucose transporter), member 2 (Slc2a2), alanyl (membrane) aminopeptidase (Anpep), transmembrane protein 27 (Tmem27), acyl-CoA synthetase medium-chain family member 2 (Acsm2), glutathione peroxidase 3 (Gpx3), fructose-1,6-biphosphatase 1 (Fbp1), alanine-glyoxylate aminotransferase 2 (Agxt2), platelet endothelial cell adhesion molecule (Pecam), and podocin (Podn).

In embodiments, the renal cell population comprises one or more cell types that express one or more of any combination of: PECAM, VEGF, KDR, HIF1a, CD31, CD146, Podocin (Podn), and Nephrin (Neph), chemokine (C-X-C motif) receptor 4 (Cxcr4), endothelin receptor type B (Ednrb), collagen, type V, alpha 2 (Col5a2), Cadherin 5 (Cdh5), plasminogen activator, tissue (Plat), angiopoietin 2 (Angpt2), kinase insert domain protein receptor (Kdr), secreted protein, acidic, cysteine-rich (osteonectin) (Sparc), serglycin (Srgn), TIMP metallopeptidase inhibitor 3 (Timp3), Wilms tumor 1 (Wt1), wingless-type MMTV integration site family, member 4 (Wnt4), regulator of G-protein signaling 4 (Rgs4), Erythropoietin (EPO).

In embodiments, the renal cell population comprises one or more cell types that express one or more of any combination of: PECAM, vEGF, KDR, HIF1a, podocin, nephrin, EPO, CK7, CK8/18/19.

In embodiments, the renal cell population comprises one or more cell types that express one or more of any combination of: PECAM, vEGF, KDR, HIF1a, CD31, CD146.

In embodiments, the renal cell population comprises one or more cell types that express one or more of any combination of: Podocin (Podn), and Nephrin (Neph).

In embodiments, the renal cell population comprises one or more cell types that express one or more of any combination of: PECAM, vEGF, KDR, HIF1a, and EPO.

In embodiments, the presence (e.g., expression) and/or level/amount of various biomarkers in a sample or cell population can be analyzed by a number of methodologies, many of which are known in the art and understood by the skilled artisan, including, but not limited to, immunohistochemical (“IHC”), Western blot analysis, immunoprecipitation, molecular binding assays, ELISA, ELIFA, fluorescence activated cell sorting (“FACS”), MassARRAY, proteomics, biochemical enzymatic activity assays, in situ hybridization, Southern analysis, Northern analysis, whole genome sequencing, polymerase chain reaction (“PCR”) including quantitative real time PCR (“qRT-PCR”) and other amplification type detection methods, such as, for example, branched DNA, SISBA, TMA and the like), RNA-Seq, FISH, microarray analysis, gene expression profiling, and/or serial analysis of gene expression (“SAGE”), as well as any one of the wide variety of assays that can be performed by protein, gene, and/or tissue array analysis. Non-limiting examples of protocols for evaluating the status of genes and gene products include Northern Blotting, Southern Blotting, Immunoblotting, and PCR Analysis. In embodiments, multiplexed immunoassays such as those available from Rules Based Medicine or Meso Scale Discovery may also be used. In embodiments, the presence (e.g., expression) and/or level/amount of various biomarkers in a sample or cell population can be analyzed by a number of methodologies, many of which are known in the art and understood by the skilled artisan, including, but not limited to, “-omics” platforms such as genome-wide transcriptomics, proteomics, secretomics, lipidomics, phospatomics, exosomics etc., wherein high-throughput methodologies are coupled with computational biology and bioinformatics techniques to elucidate a complete biological signature of genes, miRNA, proteins, secreted proteins, lipids, etc. that are expressed and not expressed by the cell population under consideration.

In embodiments, a method of detecting the presence of two or more biomarkers in a renal cell population comprises contacting the sample with an antibody directed to a biomarker under conditions permissive for binding of the antibody to its cognate ligand (i.e., biomarker), and detecting the presence of the bound antibody, e.g., by detecting whether a complex is formed between the antibody and the biomarker. In embodiments, the detection of the presence of one or more biomarkers is by immunohistochemistry. The term “detecting” as used herein encompasses quantitative and/or qualitative detection.

In embodiments, a renal cell population are identified with one or more reagents that allow detection of a biomarker disclosed herein, such as AQP1, AQP2, AQP4, Calbindin, Calponin, CD117, CD133, CD146, CD24, CD31 (PECAM-1), CD54 (ICAM-1), CD73, CK18, CK19, CK7, CK8, CK8/18, CK8/18/19, Connexin 43, Cubilin, CXCR4 (Fusin), DBA, E-cadherin (CD324), EPO (erythropoeitin), GGT1, GLEPP1 (glomerular epithelial protein 1), Haptoglobulin, Itgb1 (Integrin p), KIM-1 (kidney injury molecule-1), T1M-1 (T-cell immunoglobulin and mucin-containing molecule), MAP-2 (microtubule-associated protein 2), Megalin, N-cadherin, Nephrin, NKCC (Na-K-Cl-cotransporters), OAT-1 (organic anion transporter 1), Osteopontin, Pan-cadherin, PCLP1 (podocalyxin-like 1 molecule), Podocin, SMA (smooth muscle alpha-actin), Synaptopodin, THP (tamm-horsfall protein), Vimentin, and αGST-1 (alpha glutathione 5-transferase). In embodiments, a biomarker is detected by a monoclonal or polyclonal antibody.

In embodiments, the source of cells is the same as the intended target organ or tissue. In embodiments, BRCs or SRCs may be sourced from the kidney to be used in a formulation to be administered to the kidney. In embodiments, the cell population is derived from a kidney biopsy. In embodiments, a cell populations is derived from whole kidney tissue. In embodiments, a cell population is derived from in vitro cultures of mammalian kidney cells, established from kidney biopsies or whole kidney tissue.

In embodiments, the BRCs or SRCs comprise heterogeneous mixtures or fractions of bioactive renal cells. In embodiments, the BRCs or SRCs may be derived from or are themselves renal cell fractions from healthy individuals. In embodiments, included herein is a renal cell population or fraction obtained from an unhealthy individual that may lack certain cell types when compared to the renal cell population of a healthy individual (e.g., in a kidney or biopsy thereof). In embodiments, provided herein is a therapeutically-active cell population lacking cell types compared to a healthy individual. In embodiments, a cell population is isolated and expanded from an autologous cell population.

In embodiments, SRCs are obtained from isolation and expansion of renal cells from a patient's renal cortical tissue via a kidney biopsy. In embodiments, renal cells are isolated from the kidney tissue by enzymatic digestion, expanded using standard cell culture techniques, and selected by centrifugation across a density boundary, barrier, or interface from the expanded renal cells. In embodiments, renal cells are isolated from the kidney tissue by enzymatic digestion, expanded using standard cell culture techniques, and selected by continuous or discontinuous single or multistep density gradient centrifugation from the expanded renal cells. In embodiments, SRCs are composed primarily of renal epithelial cells which are known for their regenerative potential. In embodiments, other parenchymal (vascular) and stromal cells may be present in the autologous SRC population.

In embodiments, bioactive renal cells are obtained from renal cells isolated from kidney tissue by enzymatic digestion and expanded using standard cell culture techniques. In embodiments, the cell culture medium is designed to expand bioactive renal cells with regenerative capacity. In embodiments, the cell culture medium does not contain any recombinant or purified differentiation factors. In embodiments, the expanded heterogeneous mixtures of renal cells are cultured in hypoxic conditions to further enrich the composition of cells with regenerative capacity. Without wishing to be bound by theory, this may be due to one or more of the following phenomena: 1) selective survival, death, or proliferation of specific cellular components during the hypoxic culture period; 2) alterations in cell granularity and/or size in response to the hypoxic culture, thereby effecting alterations in buoyant density and subsequent localization during density gradient separation or during centrifugation across a density boundary, barrier, or interface; and 3) alterations in cell gene/protein expression in response to the hypoxic culture period, thereby resulting in differential characteristics of the cells within the isolated and expanded population.

In embodiments, the bioactive renal cell population is obtained from isolation and expansion of renal cells from kidney tissue (such as tissue obtained from a biopsy) under culturing conditions that enrich for cells capable of kidney regeneration.

In embodiments, renal cells from kidney tissue (such as tissue obtained from a biopsy) are passaged 1, 2, 3, 4, 5, or more times to produce expanded bioactive renal cells (such as a cell population enriched for cells capable of kidney regeneration). In embodiments, renal cells from kidney tissue (such as tissue obtained from a biopsy) are passaged 1 time to produce expanded bioactive renal cells. In embodiments, renal cells from kidney tissue (such as tissue obtained from a biopsy) are passaged 2 times to produce expanded bioactive renal cells. In embodiments, renal cells from kidney tissue (such as tissue obtained from a biopsy) are passaged 3 times to produce expanded bioactive renal cells. In embodiments, renal cells from kidney tissue (such as tissue obtained from a biopsy) are passaged 4 times to produce expanded bioactive renal cells. In embodiments, renal cells from kidney tissue (such as tissue obtained from a biopsy) are passaged 5 times to produce expanded bioactive renal cells. In embodiments, passaging the cells depletes the cell population of non-bioactive renal cells. In embodiments, passaging the cells depletes the cell population of at least one cell type. In embodiments, passaging the cells depletes the cell population of cells having a density greater than 1.095 g/ml. In embodiments, passaging the cells depletes the cell population of small cells of low granularity. In embodiments, passaging the cells depletes the cell population of cells that are smaller than erythrocytes. In embodiments, passaging the cells depletes the cell population of cells with a diameter of less than 6 μm. In embodiments, passaging cells depletes cell population of cells with a diameter less than 2 μm. In embodiments, passaging the cells depletes the cell population of cells with lower granularity than erythrocytes. In embodiments, the viability of the cell population increases after 1 or more passages. In embodiments, descriptions of small cells and low granularity are used when analyzing cells by fluorescence activated cell sorting (FACs), e.g., using the X-Y axis of a scatter-plot of where the cells show up.

In embodiments, the expanded bioactive renal cells are grown under hypoxic conditions for at least about 6, 9, 10, 12, or 24 hours but less than 48 hours, or from 6 to 9 hours, or from 6 to 48 hours, or from about 12 to about 15 hours, or about 8 hours, or about 12 hours, or about 24 hours, or about 36 hours, or about 48 hours. In embodiments, cells grown under hypoxic conditions are selected based on density. In embodiments, the bioactive renal cell population is a selected renal cell (SRC) population obtained after continuous or discontinuous (single step or multistep) density gradient separation of the expanded renal cells (e.g., after passaging and/or culture under hypoxic conditions). In embodiments, the bioactive renal cell population is a selected renal cell (SRC) population obtained after separation of the expanded renal cells by centrifugation across a density boundary, barrier, or interface (e.g., after passaging and/or culture under hypoxic condutions). In embodiments, a hypoxic culture condition is a culture condition in which cells are subjected to a reduction in available oxygen levels in the culture system relative to standard culture conditions in which cells are cultured at atmospheric oxygen levels (about 21%). In embodiments, cells cultured under hypoxic culture conditions are cultured at an oxygen level of about 5% to about 15%, or about 5% to about 10%, or about 2% to about 5%, or about 2% to about 7%, or about 2% or about 3%, or about 4%, or about 5%. In embodiments, the SRCs exhibit a buoyant density greater than approximately 1.0419 g/mL. In embodiments, the SRCs exhibit a buoyant density greater than approximately 1.04 g/mL. In embodiments, the SRCs exhibit a buoyant density greater than approximately 1.045 g/mL. In embodiments, the BRCs or SRCs contain a greater percentage of one or more cell populations and lacks or is deficient in one or more other cell populations, as compared to a starting kidney cell population.

In embodiments, expanded bioactive renal cells may be subjected to density gradient separation to obtain SRCs. In embodiments, continuous or discontinuous single step or multistep density gradient centrifugation is used to separate harvested renal cell populations based on cell buoyant density. In embodiments, expanded bioactive renal cells may be separated by centrifugation across a density boundary, barrier or interface to obtain SRCs. In embodiments, centrifugation across a density boundary or interface is used to separate harvested renal cell populations based on cell buoyant density. In embodiments, the SRCs are generated by using, in part, OPTIPREP (Axis-Shield) medium, comprising a solution of 60% (w/v) of the nonionic iodinated compound iodixanol in water. One of skill in the art, however, will recognize that other media, density gradients (continuous or discontinuous), density boundaries, barriers, interfaces or other means, e.g., immunological separation using cell surface markers known in the art, comprising necessary features for isolating cell populations described herein may be used to obtain bioactive renal cells. In embodiments, a cellular fraction exhibiting buoyant density greater than approximately 1.04 g/mL is collected after centrifugation as a distinct pellet. In embodiments, cells maintaining a buoyant density of less than 1.04 g/mL are excluded and discarded. In embodiments, a cellular fraction exhibiting buoyant density greater than approximately 1.0419 g/mL is collected after centrifugation as a distinct pellet. In embodiments, cells maintaining a buoyant density of less than 1.0419 g/mL are excluded and discarded. In embodiments, a cellular fraction exhibiting buoyant density greater than approximately 1.045 g/mL is collected after centrifugation as a distinct pellet. In embodiments, cells maintaining a buoyant density of less than 1.045 g/mL are excluded and discarded.

In embodiments, cell buoyant density is used to obtain an SRC population and/or to determine whether a renal cell population is a bioactive renal cell population. In embodiments, cell buoyant density is used to isolate bioactive renal cells. In embodiments, cell buoyant density is determined by centrifugation across a single-step OptiPrep (7% iodixanol; 60% (w/v) in OptiMEM) density interface (single step discontinuous density gradient). Optiprep is a 60% w/v solution of iodixanol in water. When used in an exemplary density interface or single step discontinuous density gradient, the Optiprep is diluted with OptiMEM (a cell culturing basal medium) to form a final solution of 7% iodixanol (in water and OptiMEM). The formulation of OptiMEM is a modification of Eagle's Minimal Essential Medium, buffered with HEPES and sodium bicarbonate, and supplemented with hypoxanthine, thymidine, sodium pyruvate, L-glutamine or GLUTAMAX, trace elements and growth factors. The protein level is minimal (15 μg/mL), with insulin and transferrin being the only protein supplements. Phenol red is included at a reduced concentration as a pH indicator. In embodiments, OptiMEM may be supplemented with 2-mercaptoethanol prior to use.

In embodiments, the OptiPrep solution is prepared and refractive index indicative of desired density is measured (R.I. 1.3456+/−0.0004) prior to use. In embodiments, renal cells are layered on top of the solution. In embodiments, the density interface or single step discontinuous density gradient is centrifuged at 800 g for 20 min at room temperature (without brake) in either a centrifuge tube (e.g., a 50 ml conical tube) or a cell processor (e.g. COBE 2991). In embodiments, the cellular fraction exhibiting buoyant density greater than approximately 1.04 g/mL is collected after centrifugation as a distinct pellet. In embodiments, cells maintaining a buoyant density of less than 1.04 g/mL are excluded and discarded. In embodiments, the cellular fraction exhibiting buoyant density greater than approximately 1.0419 g/mL is collected after centrifugation as a distinct pellet. In embodiments, cells maintaining a buoyant density of less than 1.0419 g/mL are excluded and discarded. In embodiments, the cellular fraction exhibiting buoyant density greater than approximately 1.045 g/mL is collected after centrifugation as a distinct pellet. In embodiments, cells maintaining a buoyant density of less than 1.045 g/mL are excluded and discarded. In embodiments, prior to the assessment of cell density or selection based on density, cells are cultured until they are at least 50% confluent and incubated overnight (e.g., at least about 8 or 12 hours) in a hypoxic incubator set for 2% oxygen in a 5% CO2 environment at 37° C.

In embodiments, cells obtained from a kidney sample are expanded and then processed (e.g. by hypoxia and centrifugation separation) to provide a SRC population. In embodiments, an SRC population is produced using reagents and procedures described herein. In embodiments, a sample of cells from an SRC population is tested for viability before cells of the population are administration to a subject. In embodiments, a sample of cells from an SRC population is tested for the expression of one or more of the markers disclosed herein before cells of the population administration to a subject.

Non-limiting examples of compositions and methods for preparing SRCs are disclosed in U.S. Patent Application Publication No. 2017/0281684 A1, the entire content of which is incorporated herein by reference.

In embodiments, the BRCs or SRCs are derived from a native autologous or allogeneic kidney sample. In embodiments, the BRCs or SRCs are derived from a non-autologous kidney sample. In embodiments, the sample may be obtained by kidney biopsy.

In embodiments, renal cell isolation and expansion provides a mixture of renal cell types including renal epithelial cells and stromal cells. In embodiments, SRC are obtained by continuous or discontinuous density gradient separation of the expanded renal cells. In embodiments, the primary cell type in the density gradient separated SRC population is of tubular epithelial phenotype. In embodiments, SRC are obtained by separation of the expanded renal cells by centrifugation across a density boundary, barrier, or interface. In embodiments, the primary cell type in the SRC population separated across a density boundary/barrier/interface is of tubular epithelial phenotype. In embodiments, the characteristics of SRC obtained from expanded renal cells are evaluated using a multi-pronged approach. In embodiments, cell morphology, growth kinetics and cell viability are monitored during the renal cell expansion process. In embodiments, SRC buoyant density and viability is characterized by centrifugation on or through a density gradient medium and Trypan Blue exclusion. In embodiments, SRC phenotype is characterized by flow cytometry and SRC function is demonstrated by expression of VEGF and KIM-1. In embodiments, cell function of SRC, pre-formulation, can also be evaluated by measuring the activity of two specific enzymes; GGT (γ-glutamyl transpeptidase) and LAP (leucine aminopeptidase), found in kidney proximal tubules.

In embodiments, cellular features that contribute to separation of cellular subpopulations via a density medium (size and granularity) can be exploited to separate cellular subpopulations via flow cytometry (forward scatter=a reflection of size via flow cytometry, and side scatter=a reflection of granularity). In embodiments, a density gradient or separation medium should have low toxicity towards the specific cells of interest. In embodiments, while the density medium should have low toxicity toward the specific cells of interest, the instant disclosure contemplates the use of mediums which play a role in the selection process of the cells of interest. In embodiments, and without wishing to be bound by theory, it appears that the cell populations disclosed herein recovered by the medium comprising iodixanol are iodixanol-resistant, as there is an appreciable loss of cells between the loading and recovery steps, suggesting that exposure to iodixanol under the conditions of the density gradient or density boundary, density, barrier, or density interface leads to elimination of certain cells. In embodiments, cells appearing after an iodixanol density gradient or density interface separation are resistant to any untoward effects of iodixanol and/or density gradient or interface exposure. In embodiments, a contrast medium comprising a mild to moderate nephrotoxin is used in the isolation and/or selection of a cell population, e.g. a SRC population. In embodiments, SRCs are iodixanol-resistant. In embodiments, the density medium should not bind to proteins in human plasma or adversely affect key functions of the cells of interest.

In embodiments, a cell population has been enriched and/or depleted of one or more kidney cell types using fluorescent activated cell sorting (FACS). In embodiments, kidney cell types may be enriched and/or depleted using BD FACSAria™ or equivalent. In embodiments, kidney cell types may be enriched and/or depleted using FACSAria III™ or equivalent.

In embodiments, a cell population has been enriched and/or depleted of one or more kidney cell types using magnetic cell sorting. In embodiments, one or more kidney cell types may be enriched and/or depleted using the Miltenyi autoMACS® system or equivalent.

In embodiments, a renal cell population has been subject to three-dimensional culturing. In embodiments, the methods of culturing the cell populations are via continuous perfusion. In embodiments, the cell populations cultured via three-dimensional culturing and continuous perfusion demonstrate greater cellularity and interconnectivity when compared to cell populations cultured statically. In embodiments, the cell populations cultured via three dimensional culturing and continuous perfusion demonstrate greater expression of EPO, as well as enhanced expression of renal tubule-associate genes such as E-cadherin when compared to static cultures of such cell populations. In embodiments, a cell population cultured via continuous perfusion demonstrates a greater level of glucose and glutamine consumption when compared to a cell population cultured statically.

In embodiments, low or hypoxic oxygen conditions may be used in the methods to prepare a cell population provided for herein. In embodiments, a method of preparing a cell population may be used without the step of low oxygen conditioning. In embodiments, normoxic conditions may be used.

In embodiments, a renal cell population has been isolated and/or cultured from kidney tissue. Non-limiting examples of methods are disclosed herein for separating and isolating the renal cellular components, e.g., enriched cell populations that will be used in the formulations for therapeutic use, including the treatment of kidney disease, anemia, EPO deficiency, tubular transport deficiency, and glomerular filtration deficiency. In embodiments, a cell population is isolated from freshly digested, i.e., mechanically or enzymatically digested, kidney tissue or from a heterogeneous in vitro culture of mammalian kidney cells.

In embodiments, the renal cell population comprises EPO-producing kidney cells. In embodiments, a subject has anemia and/or EPO deficiency. In embodiments, EPO-producing kidney cell populations that are characterized by EPO expression and bioresponsiveness to oxygen, such that a reduction in the oxygen tension of the culture system results in an induction in the expression of EPO. In embodiments, the EPO-producing cell populations are enriched for EPO-producing cells. In embodiments, the EPO expression is induced when the cell population is cultured under conditions where the cells are subjected to a reduction in available oxygen levels in the culture system as compared to a cell population cultured at normal atmospheric (about 21%) levels of available oxygen. In embodiments, EPO-producing cells cultured in lower oxygen conditions express greater levels of EPO relative to EPO-producing cells cultured at normal oxygen conditions. In general, the culturing of cells at reduced levels of available oxygen (also referred to as hypoxic culture conditions) means that the level of reduced oxygen is reduced relative to the culturing of cells at normal atmospheric levels of available oxygen (also referred to as normal or normoxic culture conditions). In embodiments, hypoxic cell culture conditions include culturing cells at about less than 1% oxygen, about less than 2% oxygen, about less than 3% oxygen, about less than 4% oxygen, or about less than 5% oxygen. In embodiments, normal or normoxic culture conditions include culturing cells at about 10% oxygen, about 12% oxygen, about 13% oxygen, about 14% oxygen, about 15% oxygen, about 16% oxygen, about 17% oxygen, about 18% oxygen, about 19% oxygen, about 20% oxygen, or about 21% oxygen.

In embodiments, induction or increased expression of EPO is obtained and can be observed by culturing cells at about less than 5% available oxygen and comparing EPO expression levels to cells cultured at atmospheric (about 21%) oxygen. In embodiments, the induction of EPO is obtained in a culture of cells capable of expressing EPO by a method that includes a first culture phase in which the culture of cells is cultivated at atmospheric oxygen (about 21%) for some period of time and a second culture phase in which the available oxygen levels are reduced and the same cells are cultured at about less than 5% available oxygen. In embodiments, the EPO expression that is responsive to hypoxic conditions is regulated by HIF1a. In embodiments, other oxygen manipulation culture conditions known in the art may be used for the cells described herein.

In embodiments, the formulation contains enriched populations of EPO-producing mammalian cells characterized by bio-responsiveness (e.g., EPO expression) to perfusion conditions. In embodiments, the perfusion conditions include transient, intermittent, or continuous fluid flow (perfusion). In embodiments, the EPO expression is mechanically-induced when the media in which the cells are cultured is intermittently or continuously circulated or agitated in such a manner that dynamic forces are transferred to the cells via the flow. In embodiments, the cells subjected to the transient, intermittent, or continuous fluid flow are cultured in such a manner that they are present as three-dimensional structures in or on a material that provides framework and/or space for such three-dimensional structures to form. In embodiments, the cells are cultured on porous beads and subjected to intermittent or continuous fluid flow by means of a rocking platform, orbiting platform, or spinner flask. In embodiments, the cells are cultured on three-dimensional scaffolding and placed into a device whereby the scaffold is stationary and fluid flows directionally through or across the scaffolding. Those of ordinary skill in the art will appreciate that other perfusion culture conditions known in the art may be used for the cells described herein.

In embodiments, a cell population is derived from a kidney biopsy. In embodiments, a cell population is derived from whole kidney tissue. In embodiments, a cell population is derived from an in vitro culture of mammalian kidney cells, established from kidney biopsies or whole kidney tissue. In embodiments, the renal cell population is a SRC population. In embodiments, a cell population is an unfractionated cell populations, also referred to herein as a non-enriched cell population.

Compositions containing a variety of active agents (e.g., other than renal cells) are included herein. Non-limiting examples of suitable active agents include, without limitation, cellular aggregates, acellular biomaterials, secreted products from bioactive cells, large and small molecule therapeutics, as well as combinations thereof. For example, one type of bioactive cells may be combined with biomaterial-based microcarriers with or without therapeutic molecules or another type of bioactive cells. In embodiments, unattached cells may be combined with acellular particles.

In embodiments, cells of the renal cell population are within spheroids. In embodiments, the renal cell population is in the form of spheroids. In embodiments, spheroids comprising bioactive renal cells are administered to a subject. In embodiments, the spheroids comprise at least one non-renal cell type or population of cells. In embodiments, the a spheroids are produced in a method comprising (i) combining a bioactive renal cell population and a non-renal cell population, and (ii) culturing the bioactive renal cell population and the non-renal cell population in a 3-dimensional culture system comprising a spinner flask until the spheroids form.

In embodiments, the non-renal cell population comprises an endothelial cell population or an endothelial progenitor cell population. In embodiments, the bioactive cell population is an endothelial cell population. In embodiments, the endothelial cell population is a cell line. In embodiments, the endothelial cell population comprises human umbilical vein endothelial cells (HUVECs). In embodiments, the non-renal cell population is a mesenchymal stem cell population. In embodiments, the non-renal cell population is a stem cell population of hematopoietic, mammary, intestinal, placental, lung, bone marrow, blood, umbilical cord, endothelial, dental pulp, adipose, neural, olfactory, neural crest, or testicular origin. In embodiments, the non-renal cell population is an adipose-derived progenitor cell population. In embodiments, the cell populations are xenogeneic, syngeneic, allogeneic, autologous or combinations thereof. In embodiments, the bioactive renal cell population and non-renal cell population are cultured at a ratio of from 0.1:9.9 to 9.9:0.1. In embodiments, the bioactive renal cell population and non-renal cell population are cultured at a ratio of about 1:1. In embodiments, the renal cell population and bioactive cell population are suspended in growth medium.

The expanded bioactive renal cells may be further subjected to continuous or discontinuous density medium separation to obtain the SRC. Specifically, continuous or discontinuous single step or multistep density gradient centrifugation is used to separate harvested renal cell populations based on cell buoyant density. In embodiments, the expanded bioactive renal cells may be further subjected to separation by centrifugation across a density boundary, barrier, or interface to obtain the SRC. Specifically, centrifugation across a density boundary, barrier, or interface is used to separate harvested renal cell populations based on cell buoyant density. In embodiments, the SRC are generated by using, in part, the OPTIPREP (Axis-Shield) medium, comprising a 60% solution of the nonionic iodinated compound iodixanol in water. One of skill in the art, however, will recognize that any density gradient medium without limitation of specific medium or other means, e.g., immunological separation using cell surface markers known in the art, comprising necessary features for isolating the cell populations encompassed by the instant invention may be used. For example, Percoll or sucrose may be used to form a density gradient or density boundary. In embodiments, the cellular fraction exhibiting buoyant density greater than approximately 1.04 g/mL is collected after centrifugation as a distinct pellet. In embodiments, cells maintaining a buoyant density of less than 1.04 g/mL are excluded and discarded. In embodiments, the cellular fraction exhibiting buoyant density greater than approximately 1.0419 g/mL is collected after centrifugation as a distinct pellet. In embodiments, cells maintaining a buoyant density of less than 1.0419 g/mL are excluded and discarded. In embodiments, the cellular fraction exhibiting buoyant density greater than approximately 1.045 g/mL is collected after centrifugation as a distinct pellet. In embodiments, cells maintaining a buoyant density of less than 1.045 g/mL are excluded and discarded.

The therapeutic compositions and formulations thereof may contain isolated, heterogeneous populations of kidney cells, and/or admixtures thereof, enriched for specific bioactive components or cell types and/or depleted of specific inactive or undesired components or cell types for use in the treatment of kidney disease, i.e., providing stabilization and/or improvement and/or regeneration of kidney function and/or structure, for example a previously described in Presnell et al. U.S. Pat. No. 8,318,484 and Ilagan et al. PCT/US2011/036347, the entire contents of which are incorporated herein by reference. The compositions may contain isolated renal cell fractions that lack cellular components as compared to a healthy individual yet retain therapeutic properties, i.e., provide stabilization and/or improvement and/or regeneration of kidney function. The cell populations, cell fractions, and/or admixtures of cells described herein may be derived from healthy individuals, individuals with a kidney disease, or subjects as described herein.

The disclosure contemplates therapeutic compositions of selected renal cell populations that are to be administered to target organs or tissue in a subject in need. A bioactive selected renal cell population generally refers to a cell population potentially having therapeutic properties upon administration to a subject. For example, upon administration to a subject in need, a bioactive renal cell population can provide stabilization and/or improvement and/or repair and/or regeneration of kidney function in the subject. The therapeutic properties may include a regenerative effect.

In embodiments, the source of cells is the same as the intended target organ or tissue. For example, BRCs and/or SRCs may be sourced from the kidney to be used in a formulation to be administered to the kidney. In embodiments, the cell populations are derived from a kidney biopsy. In embodiments, the cell populations are derived from whole kidney tissue. In one other embodiment, the cell populations are derived from in vitro cultures of mammalian kidney cells, established from kidney biopsies or whole kidney tissue. In embodiments, the BRCs and/or SRCs comprise heterogeneous mixtures or fractions of bioactive renal cells. The BRCs and/or SRCs may be derived from or are themselves renal cell fractions from healthy individuals. In addition, the present disclosure provides renal cell fractions obtained from an unhealthy individual that may lack certain cellular components when compared to the corresponding renal cell fractions of a healthy individual, yet still retain therapeutic properties. The present disclosure also provides therapeutically-active cell populations lacking cellular components compared to a healthy individual, which cell populations can be, In embodiments, isolated and expanded from autologous sources in various disease states.

In embodiments, the SRCs are obtained from isolation and expansion of renal cells from a patient's renal cortical tissue via a kidney biopsy. Renal cells are isolated from the kidney tissue by enzymatic digestion, expanded using standard cell culture techniques, and selected by centrifugation of the expanded renal cells across a density boundary, barrier, or interface. In this embodiment, SRC are composed primarily of renal tubular epithelial cells which are known for their regenerative potential (Bonventre JV. Dedifferentiation and proliferation of surviving epithelial cells in acute renal failure. J Am Soc Nephrol. 2003; 14(Suppl. 1):S55-61; Humphreys BD, Czerniak S, DiRocco D P, et al. Repair of injured proximal tubule does not involve specialized progenitors. PNAS. 2011; 108:9226-31; Humphreys BD, Valerius M T, Kobayashi A, et al. Intrinsic epithelial cells repair the kidney after injury. Cell Stem Cell. 2008; 2:284-91). Other parenchymal (vascular) and stromal cells may be present in the autologous SRC population. In embodiments, renal cells are selected by centrifugation through a continuous or discontinuous single step or multistep gradient.

As described herein, the present invention is based, in part, on the surprising finding that certain subfractions of a heterogeneous population of renal cells, enriched for bioactive components and depleted of inactive or undesired components, provide superior therapeutic and regenerative outcomes than the starting population.

Renal cell isolation and expansion provides a mixture of renal cell types including renal tubular epithelial cells and stromal cells. As noted above, SRC are obtained by separation of the expanded renal cells by centrifugation across a density boundary, barrier, or interface. The primary cell type in the separated SRC population is of tubular epithelial phenotype. The characteristics of SRC obtained from expanded renal cells is evaluated using a multi-pronged approach. Cell morphology, growth kinetics and cell viability are monitored during the renal cell expansion process. SRC buoyant density and viability is characterized by density interface and Trypan Blue exclusion. SRC phenotype is characterized by flow cytometry and SRC function is demonstrated by expression of VEGF and KIM-1.

Those of ordinary skill in the art will appreciate that other methods of isolation and culturing known in the art may be used for the cells described herein. Those of ordinary skill in the art will also appreciate that bioactive cell populations may be derived from sources other than those specifically listed above, including, without limitation, tissues and organs other than the kidney, body fluids and adipose.

In embodiments, one or more of a variety of biomaterials may be combined with an active agent (such as a renal cell population, a product thereof, or a spheroid comprising a renal cell population and one or more non-renal cell types or populations) to provide a therapeutic formulations. In embodiments, the biomaterials may be in any suitable shape (e.g., beads) or form (e.g., liquid, gel, etc.). Non-limiting examples of suitable biomaterials in the form of polymeric matrices are described in Bertram et al. U.S. Published Application 20070276507 (incorporated herein by reference in its entirety). In embodiments, the polymeric matrix may be a biocompatible material formed from a variety of synthetic or naturally-occurring materials including, but not limited to, open-cell polylactic acid (OPLA®), cellulose ether, cellulose, cellulosic ester, fluorinated polyethylene, phenolic, poly-4-methylpentene, polyacrylonitrile, polyamide, polyamideimide, polyacrylate, polybenzoxazole, polycarbonate, polycyanoarylether, polyester, polyestercarbonate, polyether, polyetheretherketone, polyetherimide, polyetherketone, polyethersulfone, polyethylene, polyfluoroolefin, polyimide, polyolefin, polyoxadiazole, polyphenylene oxide, polyphenylene sulfide, polypropylene, polystyrene, polysulfide, polysulfone, polytetrafluoroethylene, polythioether, polytriazole, polyurethane, polyvinyl, polyvinylidene fluoride, regenerated cellulose, silicone, urea-formaldehyde, collagens, gelatin, alginate, laminins, fibronectin, silk, elastin, alginate, hyaluronic acid, agarose, or copolymers or physical blends thereof. In embodiments, the biomaterial is a hydrogel. Scaffolding configurations may range from soft porous scaffolds to rigid, shape-holding porous scaffolds. In embodiments, a scaffold is configured as a liquid solution that is capable of becoming a hydrogel, e.g., hydrogel that is above a melting temperature.

In embodiments, the scaffold is derived from an existing kidney or other organ of human or animal origin, where the native cell population has been eliminated through application of detergent and/or other chemical agents and/or other enzymatic and/or physical methodologies known to those of ordinary skill in the art. In this embodiment, the native three dimensional structure of the source organ is retained together with all associated extracellular matrix components in their native, biologically active context. In embodiments, the scaffold is extracellular matrix derived from human or animal kidney or other organ. In embodiments, the configuration is assembled into a tissue-like structure through application of three dimensional bioprinting methodologies. In embodiments, the configuration is the liquid form of a solution that is capable of becoming a hydrogel.

Hydrogels may be formed from a variety of polymeric materials and are useful in a variety of biomedical applications. Hydrogels can be described physically as three-dimensional networks of hydrophilic polymers. Depending on the type of hydrogel, they contain varying percentages of water, but altogether do not dissolve in water. Despite their high water content, hydrogels are capable of additionally binding great volumes of liquid due to the presence of hydrophilic residues. Hydrogels swell extensively without changing their gelatinous structure. Hydrogels swell extensively without changing their gelatinous structure. The basic physical features of a hydrogel can be specifically modified, according to the properties of the polymers used and the device used to administer the hydrogel.

In embodiments, a hydrogel is formed when an organic polymer (e.g., natural or synthetic) is crosslinked via covalent, ionic, or hydrogen bonds to create a three-dimensional open-lattice structure which entraps water molecules to form a gel. In embodiments, the material used to form a hydrogel includes a polysaccharide such as alginate, polyphosphazines, and polyacrylates, which are crosslinked tonically, or block copolymers such as Pluronics™ or Tetronics™, polyethylene oxide-polypropylene glycol block copolymers which are crosslinked by temperature or pH, respectively. In embodiments, a hydrogel comprises gelatin (e.g., the hydrogel is a biodegradable gelatin-based hydrogel). In embodiments, the hydrogel material does not induce an inflammatory response.

Non-limiting examples of other materials which can be used to form a hydrogel include (a) modified alginates, (b) polysaccharides (e.g. gellan gum and carrageenans) which gel by exposure to monovalent cations, (c) polysaccharides (e.g., hyaluronic acid) that are very viscous liquids or are thixotropic and form a gel over time by the slow evolution of structure, (d) gelatin or collagen, and (e) polymeric hydrogel precursors (e.g., polyethylene oxide-polypropylene glycol block copolymers and proteins). U.S. Pat. No. 6,224,893 B1 provides a detailed description of various polymers, and the chemical properties of such polymers, that are suitable for making hydrogels in accordance with certain embodiments described herein.

In embodiments, the hydrogel used to formulate a biomaterial is gelatin-based. Gelatin is a non-toxic, biodegradable and water-soluble protein derived from collagen, which is a major component of mesenchymal tissue extracellular matrix (ECM). Gelatin retains informational signals including an arginine-glycine-aspartic acid (RGD) sequence, which promotes cell adhesion, proliferation and stem cell differentiation. A characteristic property of gelatin is that it exhibits Upper Critical Solution Temperature behavior (UCST). In embodiments, above a specific temperature threshold of 40° C., gelatin can be dissolved in water by the formation of flexible, random single coils. Upon cooling, hydrogen bonding and Van der Waals interactions occur, resulting in the formation of triple helices. In embodiments, these collagen-like triple helices act as junction zones and thus trigger the sol-gel transition. Gelatin is widely used in pharmaceutical and medical applications.

Collagen is the main structural protein in the extracellular space in the various connective tissues in animal bodies. As the main component of connective tissue, it is the most abundant protein in mammals, making up from 25% to 35% of the whole-body protein content. Depending upon the degree of mineralization, collagen tissues may be rigid (bone), compliant (tendon), or have a gradient from rigid to compliant (cartilage). Collagen, in the form of elongated fibrils, is mostly found in fibrous tissues such as tendons, ligaments and skin. It is also abundant in corneas, cartilage, bones, blood vessels, the gut, intervertebral discs and the dentin in teeth. In muscle tissue, it serves as a major component of the endomysium. Collagen constitutes one to two percent of muscle tissue, and accounts for 6% of the weight of strong, tendinous muscles. Collagen occurs in many places throughout the body. Over 90% of the collagen in the human body, however, is type I.

To date, 28 types of collagen have been identified and described. They can be divided into several groups according to the structure they form: Fibrillar (Type I, II, III, V, XI). Non-fibrillar FACIT (Fibril Associated Collagens with Interrupted Triple Helices) (Type IX, XII, XIV, XVI, XIX). Short chain (Type VIII, X). Basement membrane (Type IV). Multiplexin (Multiple Triple Helix domains with Interruptions) (Type XV, XVIII). MACIT (Membrane Associated Collagens with Interrupted Triple Helices) (Type XIII, XVII). Other (Type VI, VII). The five most common types are: Type I: skin, tendon, vascular ligature, organs, bone (main component of the organic part of bone). Type II: cartilage (main collagenous component of cartilage) Type III: reticulate (main component of reticular fibers), commonly found alongside type I. Type IV: forms basal lamina, the epithelium-secreted layer of the basement membrane. Type V: cell surfaces, hair and placenta.

Gelatin retains informational signals including an arginine-glycine-aspartic acid (RGD) sequence, which promotes cell adhesion, proliferation and stem cell differentiation. A characteristic property of gelatin is that it exhibits Upper Critical Solution Temperature behavior (UCST). Above a specific temperature threshold of 40° C., gelatin can be dissolved in water by the formation of flexible, random single coils. Upon cooling, hydrogen bonding and Van der Waals interactions occur, resulting in the formation of triple helices. These collagen-like triple helices act as junction zones and thus trigger the sol-gel transition. Gelatin is widely used in pharmaceutical and medical applications.

In embodiments, the hydrogel used to formulate the injectable cell compositions herein is based on porcine gelatin, which may be sourced from porcine skin and is commercially available, for example from Nitta Gelatin NA Inc (NC, USA) or Gelita USA Inc. (IA, USA). Gelatin may be dissolved, for example, in Dulbecco's phosphate-buffered saline (DPBS) to form a thermally responsive hydrogel, which can gel and liquefy at different temperatures. In embodiments, the hydrogel used to formulate the injectable cell compositions herein is based on recombinant human or animal gelatin expressed and purified using methodologies known to those of ordinary skill in the art. In embodiments, an expression vector containing all or part of the cDNA for Type I, alpha I human collagen is expressed in the yeast Pichia pastoris. Other expression vector systems and organisms will be known to those of ordinary skill in the art. In a particular embodiment, the gelatin-based hydrogel may be one that is liquid at and above room temperature (22-28° C.) and that gels when cooled to refrigerated temperatures (2-8° C.).

In embodiments, the gelatin-based hydrogel biomaterial used to formulate SRC into NKA is a porcine gelatin dissolved in buffer to form a thermally responsive hydrogel. In embodiments, this hydrogel is fluid at room temperature but gels when cooled to refrigerated temperature (2-8° C.). SRC are formulated with the hydrogel to obtain NKA. In embodiments, NKA is gelled by cooling and is shipped to the clinic under refrigerated temperature (2-8° C.). In embodiments, NKA has a shelf life of 3 days. In embodiments, at the clinical site, the product is warmed to room temperature before injecting into the patient's kidney. In embodiments, NKA is implanted into the kidney cortex using a needle and syringe suitable for delivery of NKA via a percutaneous or laparoscopic procedure. In embodiments, the hydrogel is derived from gelatin or another extracellular matrix protein of recombinant origin. In embodiments, the hydrogel is derived from extracellular matrix sourced from kidney or another tissue or organ. In embodiments, the hydrogel is derived from a recombinant extracellular matrix protein. In embodiments, the hydrogel comprises gelatin derived from recombinant collagen (i.e., recombinant gelatin).

In embodiments, scaffolding or biomaterial characteristics may enable cells to attach and interact with the scaffolding or biomaterial material, and/or may provide porous spaces into which cells can be entrapped. In embodiments, the porous scaffolds or biomaterials allow for the addition or deposition of one or more populations of cells on a biomaterial configured as a porous scaffold (e.g., by attachment of the cells) and/or within the pores of the scaffold (e.g., by entrapment of the cells). In embodiments, the scaffolds or biomaterials allow or promote for cell:cell and/or cell:biomaterial interactions within the scaffold to form constructs as described herein.

In embodiments, the biomaterial is comprised of hyaluronic acid (HA) in hydrogel form, containing HA molecules ranging in size from 5.1 kDA to >2×10⁶ kDa. In embodiments, the biomaterial is comprised of hyaluronic acid in porous foam form, also containing HA molecules ranging in size from 5.1 kDA to >2×10⁶ kDa. In embodiments, the biomaterial is comprised of a poly-lactic acid (PLA)-based foam, having an open-cell structure and pore size of about 50 microns to about 300 microns. In embodiments, a renal cell population provides directly and/or stimulate synthesis of high molecular weight Hyaluronic Acid through Hyaluronic Acid Synthase-2 (HAS-2), especially after intra-renal implantation.

In embodiments, the biomaterials described herein respond to certain external conditions, e.g., in vitro or in vivo. In embodiments, the biomaterials are temperature-sensitive (e.g., either in vitro or in vivo). In embodiments, the biomaterials respond to exposure to enzymatic degradation (e.g., either in vitro or in vivo). In embodiments, a biomaterial's response to external conditions can be fine-tuned as described herein. In embodiments, temperature sensitivity of the formulation described can be varied by adjusting the percentage of a biomaterial in the formulation. For example, the percentage of gelatin in a solution can be adjusted to modulate the temperature sensitivity of the gelatin in the final formulation (e.g., liquid, gel, beads, etc.). In embodiments, the gelatin solution may be provided in PBS, DMEM, or another suitable solvent. In embodiments, biomaterials may be chemically crosslinked to provide greater resistance to enzymatic degradation. For instance, a carbodiimide crosslinker may be used to chemically crosslink gelatin beads thereby providing a reduced susceptibility to endogenous enzymes.

In embodiments, the response by the biomaterial to external conditions concerns the loss of structural integrity of the biomaterial. Although temperature-sensitivity and resistance to enzymatic degradation are provided herein, other mechanisms exist by which the loss of material integrity may occur in different biomaterials. These mechanisms may include, but are not limited to, thermodynamic (e.g., a phase transition such as melting, diffusion (e.g., diffusion of an ionic crosslinker from a biomaterial into the surrounding tissue)), chemical, enzymatic, pH (e.g., pH-sensitive liposomes), ultrasound, and photolabile (light penetration). In embodiments, the exact mechanism by which the biomaterial loses structural integrity will vary but typically the mechanism is triggered either at the time of implantation or post-implantation.

In embodiments, the formulations described herein incorporate biomaterials having properties which create a favorable environment for the active agent (such as a renal cell population, a product thereof, or a spheroid comprising a renal cell population and one or more non-renal cell types or populations) to be administered to a subject. In embodiments, the formulation contains a first biomaterial that provides a favorable environment from the time the active agent is formulated with the biomaterial up until the point of administration to the subject. In embodiments, the favorable environment concerns the advantages of having one or more active agents (such as a renal cell population, a product thereof, or a spheroid comprising a renal cell population and one or more non-renal cell types or populations) suspended in a substantially solid state versus a fluid (as described herein) prior to administration to a subject. In embodiments, the first biomaterial is a temperature-sensitive biomaterial. In embodiments, the temperature-sensitive biomaterial may have (i) a substantially solid state at about 8° C. or below, and (ii) a substantially liquid state at ambient temperature or above. In embodiments, the ambient temperature refers to the temperature at which a composition will be administered. In embodiments, the ambient temperature is the temperature of a temperature-controlled environment. In embodiments, the ambient temperature is about room temperature. In embodiments, ambient temperature ranges from about 18° C. to about 30° C. In embodiments, ambient temperature is about 18° C., about 19° C., about 20° C., about 21° C., about 22° C., about 23° C., about 24° C., about 25° C., about 26° C., about 27° C., about 28° C., about 29° C., or about 30° C. In embodiments, one or more active agents (such as a renal cell population, a product thereof, or a spheroid comprising a renal cell population and one or more non-renal cell types or populations) described herein may be coated with, deposited on, embedded in, attached to, seeded, suspended in, or entrapped in a temperature-sensitive biomaterial.

In embodiments, one or more active agents (such as a renal cell population, a product thereof, or a spheroid comprising a renal cell population and one or more non-renal cell types or populations) is uniformly dispersed throughout the volume of the cell-stabilizing biomaterial.

In embodiments, the formulation is an injectable formulation comprising one or more active agents (such as a renal cell population, a product thereof, or a spheroid comprising a renal cell population and one or more non-renal cell types or populations) and a temperature-sensitive cell-stabilizing biomaterial that maintains (i) a substantially solid state at 8° C. or below, and (ii) a substantially liquid state at ambient temperature or above, wherein the biomaterial comprises a hydrogel, wherein the biomaterial is in a solid-to-liquid transitional stage between 8° C. and ambient temperature or above; and wherein the one or more active agents is suspended in and dispersed throughout the cell-stabilizing biomaterial. In embodiments, the ambient temperature ranges from 18° C. to 30° C. In embodiments, the biomaterial is in a liquid state at 37° C. In embodiments, the substantially solid state is a gel state. In embodiments, the hydrogel comprises gelatin. In embodiments, the gelatin is present in the formulation at 0.5% to 1% (w/v). In embodiments, the gelatin is present in the formulation at 0.75% (w/v).

In embodiments, the formulation further comprises an antioxidant, an oxygen carrier, an immunomodulatory factor, a cell recruitment factor, a cell attachment factor, an anti-inflammatory agent, an immunosuppressant, an angiogenic factor, or a wound healing factor.

In embodiments, the formulation further comprises an antioxidant. In embodiments, the antioxidant is 6-hydroxy-2,5,7,8-tetramethylchroman-2-carboxylic acid. In embodiments, the 6-hydroxy-2,5,7,8-tetramethylchroman-2-carboxylic acid is present at 50 μM to 150 μM. In embodiments, the 6-hydroxy-2,5,7,8-tetramethylchroman-2-carboxylic acid is present at 100 μM.

In embodiments, the formulation further comprises an oxygen carrier. In embodiments, the oxygen carrier is a perfluorocarbon.

In embodiments, the formulation further comprises an immunomodulatory factor.

In embodiments, the formulation further comprises an immunosuppressant.

In embodiments, the formulation comprises 0.75% (w/v) gelatin and 100 μM 6-hydroxy-2,5,7,8-tetramethylchroman-2-carboxylic acid.

In embodiments, the formulation further comprises biocompatible beads comprising a biomaterial. In embodiments, the beads are crosslinked. In embodiments, the crosslinked beads have a reduced susceptibility to enzymatic degradation as compared to non-crosslinked biocompatible beads. In embodiments, the crosslinked beads are carbodiimide-crosslinked beads. In embodiments, the carbodiimide is selected from the group consisting of 1-Ethyl-3-[3-dimethylaminopropyl] carbodiimide hydrochloride (EDC), DCC-N,N′-dicyclohexylcarbodiimide (DCC), and N,N′-Diisopropylcarbodiimide (DIPC). In embodiments, the carbodiimide is 1-Ethyl-3-[3-dimethylaminopropyl] carbodiimide hydrochloride (EDC). In embodiments, the crosslinked beads comprise a reduced number of free primary amines as compared to non-crosslinked beads. In embodiments, the number of free primary amines is detectable spectrophotometrically at 355 nm. In embodiments, the beads are seeded with the active agent (such as a renal cell population, a product thereof, or a spheroid comprising a renal cell population and one or more non-renal cell types or populations). In embodiments, the formulation further comprises additional biocompatible beads that comprise a temperature-sensitive biomaterial that maintains (i) a substantially solid state at ambient temperature or below, and (ii) a substantially liquid state at 37° C. or above. In embodiments, the biomaterial comprises a solid-to-liquid transitional state between ambient temperature and 37° C. In embodiments, the substantially solid state is a gel state. In embodiments, the biomaterial comprises a hydrogel. In embodiments, the hydrogel comprises gelatin. In embodiments, the beads comprise gelatin at 5% (w/v) to 10% (w/v). In embodiments, the additional biocompatible beads are spacer beads. In embodiments, the spacer beads are not seeded with active agent (such as a renal cell population, a product thereof, or a spheroid comprising a renal cell population and one or more non-renal cell types or populations).

In embodiments, the formulation comprises or further comprises a product secreted by a renal cell population. In embodiments, the product comprises a paracrine factor. In embodiments, the product comprises an endocrine factor. In embodiments, the product comprises a juxtacrine factor. In embodiments, the products comprise vesicles. In embodiments, the vesicles comprise microvesicles. In embodiments, the vesicles comprise exosomes.

In embodiments, the vesicles comprise a secreted product selected from the group consisting of paracrine factors, endocrine factors, juxtacrine factors, and RNA. In embodiments, the RNA is an miRNA. In embodiments, the vesicles comprise an miRNA that inhibits Plasminogen Activation Inhibitor-1 (PAI-1) and/or TGFβ1.

In embodiments, the secreted product that comprises a paracrine and/or juxtacrine factor, such as alpha-1 microglobulin, beta-2-microglobulin, calbindin, clusterin, connective tissue growth factor, cystatin-C, glutathione-S-transferase alpha, kidney injury moleculte-1, neutraphil gelatinase-associated lipocalin, osteopontin, trefoil factor 3, tam-horsfall urinary glycoprotein, tissue-inhibitor of metallo proteinase 1, vascular endothelial growth factor, fibronectin, interleukin-6, or monocyte chemotactic protein-1.

Further included by the disclosure herein are formulations that contain biomaterials which degrade over a period time on the order of seconds, minutes, hours, or days. This is in contrast to a large body of work focusing on the implantation of solid materials that then slowly degrade over days, weeks, or months. In embodiments, the biomaterial has one or more of the following characteristics: biocompatibility, biodegradeable/bioresorbable, a substantially solid state prior to and during implantation into a subject, loss of structural integrity (substantially solid state) after implantation, and cytocompatible environment to support cellular viability. In embodiments, the biomaterial's ability to keep implanted particles spaced out during implantation enhances native tissue ingrowth. In embodiments, the biomaterial also facilitates implantation of solid formulations. In embodiments, the biomaterial provides for localization of the formulation described herein since inserted of a solid unit helps prevent the delivered materials from dispersing within the tissue during implantation. In embodiments, for cell-based formulations, a solid biomaterial also improves stability and viability of anchorage dependent cells compared to cells suspended in a fluid. In embodiments, a short duration of the structural integrity means that soon after implantation, the biomaterial does not provide a significant barrier to tissue ingrowth or integration of the delivered cells/materials with host tissue.

In embodiments, a construct includes a biomaterial configured as a three-dimensional (3D) porous biomaterial suitable for entrapment and/or attachment of the admixture. In embodiments, a construct includes a biomaterial configured as a liquid or semi-liquid gel suitable for embedding, attaching, suspending, or coating mammalian cells. In embodiments, a construct includes a biomaterial configured comprised of a predominantly high-molecular weight species of hyaluronic acid (HA) in hydrogel form. In embodiments, a construct includes a biomaterial comprised of a predominantly high-molecular weight species of hyaluronic acid in porous foam form. In embodiments, a construct includes a biomaterial comprised of a poly-lactic acid-based foam having pores of between about 50 microns to about 300 microns. In embodiments, a construct includes one or more cell populations that may be derived from a kidney sample that is autologous to the subject in need of improved kidney function. In embodiments, a the sample is a kidney biopsy. In embodiments, a the subject has a kidney disease. In embodiments, a the cell population is derived from a non-autologous kidney sample. In embodiments, a construct provides increased renal function. In embodiments, a construct provides kidney regeneration. In embodiments, a construct provides erythroid homeostasis.

In embodiments, a formulation contains bioactive cells combined with a second biomaterial that provides a favorable environment for the combined cells from the time of formulation up until a point after administration to the subject. In embodiments, the favorable environment provided by the second biomaterial concerns the advantages of administering cells in a biomaterial that retains structural integrity up until the point of administration to a subject and for a period of time after administration. In embodiments, the structural integrity of the second biomaterial following implantation is minutes, hours, days, or weeks. In embodiments, the structural integrity is less than one month, less than one week, less than one day, or less than one hour. In embodiments, the relatively short term structural integrity provides a formulation that can deliver the active agent and biomaterial to a target location in a tissue or organ with controlled handling, placement or dispersion without being a hindrance or barrier to the interaction of the incorporated elements with the tissue or organ into which it was placed.

In embodiments, the second biomaterial is a temperature-sensitive biomaterial that has a different sensitivity than the first biomaterial. The second biomaterial may have (i) a substantially solid state at about ambient temperature or below, and (ii) a substantially liquid state at about 37° C. or above. In embodiments, the ambient temperature is about room temperature.

In embodiments, the second biomaterial is crosslinked beads. In embodiments, the crosslinked beads may have finely tunable in vivo residence times depending on the degree of crosslinking, as described herein. In embodiments, the crosslinked beads comprise bioactive cells and are resistant to enzymatic degradation as described herein.

In embodiments, the formulations of the present disclosure may include the first biomaterial combined with an active agent, e.g., bioactive cells, with or without a second biomaterial combined with an active agent, e.g., bioactive cells. In embodiments, where a formulation includes a second biomaterial, it may be a temperature sensitive bead and/or a crosslinked bead.

In embodiments, the bioactive cell preparations and/or constructs described herein can be administered as bioactive cell formulations. In embodiments, the formulations include the cells and one or more biomaterials that provide stability to the bioactive cell preparations and/or constructs described herein. In embodiments, the biomaterial is a temperature-sensitive biomaterial that can maintain at least two different phases or states depending on temperature. In embodiments, the biomaterial is capable of maintaining a first state at a first temperature, a second state at a second temperature, and/or a third state at a third temperature. In embodiments, the first, second or third state may be a substantially solid, a substantially liquid, or a substantially semi-solid or semi-liquid state. In embodiments, the biomaterial has a first state at a first temperature and a second state at a second temperature, wherein the first temperature is lower than the second temperature.

In embodiments, the state of a temperature-sensitive biomaterial is a substantially solid state at a temperature of about 8° C. or below. In embodiments, the substantially solid state is maintained at about 1° C., about 2° C., about 3° C., about 4° C., about 5° C., about 6° C., about 7° C., or about 8° C. In embodiments, the substantially solid state has the form of a gel. In embodiments, the state of the temperature-sensitive biomaterial is a substantially liquid state at ambient temperature or above. In embodiments, the substantially liquid state is maintained at about 25° C., about 25.5° C., about 26° C., about 26.5° C., about 27° C., about 27.5° C., about 28° C., about 28.5° C., about 29° C., about 29.5° C., about 30° C., about 31° C., about 32° C., about 33° C., about 34° C., about 35° C., about 36° C., or about 37° C. In embodiments, the ambient temperature is about room temperature.

In embodiments, the state of a temperature-sensitive biomaterial is a substantially solid state at a temperature of about ambient temperature or below. In embodiments, the ambient temperature is about room temperature. In embodiments, the substantially solid state is maintained at about 17° C., about 16° C., about 15° C., about 14° C., about 13° C., about 12° C., about 11° C., about 10° C., about 9° C., about 8° C., about 7° C., about 6° C., about 5° C., about 4° C., about 3° C., about 2° C., or about 1° C. In embodiments, the substantially solid state has the form of a bead. In embodiments, the state of the temperature-sensitive biomaterial is a substantially liquid state at a temperature of about 37° C. or above. In embodiments, the substantially solid state is maintained at about 37° C., about 38° C., about 39° C., or about 40° C.

In embodiments, a temperature-sensitive biomaterial may be provided in the form of a solution, in the form of beads, or in other suitable forms described herein and/or known to those of ordinary skill in the art. In embodiments, the cell populations and preparations described herein may be coated with, deposited on, embedded in, attached to, seeded, suspended in, or entrapped in a temperature-sensitive biomaterial. In embodiments, the temperature-sensitive biomaterial may be provided without any cells, such as, for example in the form of spacer beads.

In embodiments, the temperature-sensitive biomaterial has a transitional state between a first state and a second state. In embodiments, the transitional state is a solid-to-liquid transitional state between a temperature of about 8° C. and about ambient temperature. In embodiments, the ambient temperature is about room temperature. In embodiments, the solid-to-liquid transitional state occurs at one or more temperatures of about 8° C., about 9° C., about 10° C., about 11° C., about 12° C., about 13° C., about 14° C., about 15° C., about 16° C., about 17° C., and about 18° C.

In embodiments, a temperature-sensitive biomaterial has a certain viscosity at a given temperature measured in centipoise (cP). In embodiments, the biomaterial has a viscosity at 25° C. of about 1 cP to about 5 cP, about 1.1 cP to about 4.5 cP, about 1.2 cP to about 4 cP, about 1.3 cP to about 3.5 cP, about 1.4 cP to about 3.5 cP, about 1.5 cP to about 3 cP, about 1.55 cP to about 2.5 cP, or about 1.6 cP to about 2 cP. In embodiments, the biomaterial has a viscosity at 37° C. of about 1.0 cP to about 1.15 cP. The viscosity at 37° C. may be about 1.0 cP, about 1.01 cP, about 1.02 cP, about 1.03 cP, about 1.04 cP, about 1.05 cP, about 1.06 cP, about 1.07 cP, about 1.08 cP, about 1.09 cP, about 1.10 cP, about 1.11 cP, about 1.12 cP, about 1.13 cP, about 1.14 cP, or about 1.15 cP. In embodiments, the biomaterial is a gelatin solution. In embodiments, the gelatin is present at about 0.5%, about 0.55%, about 0.6%, about 0.65%, about 0.7%, about 0.75%, about 0.8%, about 0.85%, about 0.9%, about 0.95% or about 1%, (w/v) in the solution. In embodiments, the biomaterial is a 0.75% (w/v) gelatin solution in PBS. In embodiments, the 0.75% (w/v) solution has a viscosity at 25° C. of about 1.6 cP to about 2 cP. In embodiments, the 0.75% (w/v) solution has a viscosity at 37° C. of about 1.07 cP to about 1.08 cP. In embodiments, the gelatin solution may be provided in PBS, DMEM, or another suitable solvent.

In embodiments, the bioactive cell formulation also includes a cell viability agent. In embodiments, the cell viability agent is selected from the group consisting of an antioxidant, an oxygen carrier, an immunomodulatory factor, a cell recruitment factor, a cell attachment factor, an anti-inflammatory agent, an angiogenic factor, a matrix metalloprotease, a wound healing factor, and products secreted from bioactive cells.

In embodiments, antioxidants are characterized by the ability to inhibit oxidation of other molecules. Antioxidants include, without limitation, one or more of 6-hydroxy-2,5,7,8-tetramethylchroman-2-carboxylic acid (Trolox®), carotenoids, flavonoids, isoflavones, ubiquinone, glutathione, lipoic acid, superoxide dismutase, ascorbic acid, vitamin E, vitamin A, mixed carotenoids (e.g., beta carotene, alpha carotene, gamma carotene, lutein, lycopene, phytopene, phytofluene, and astaxanthin), selenium, Coenzyme Q10, indole-3-carbinol, proanthocyanidins, resveratrol, quercetin, catechins, salicylic acid, curcumin, bilirubin, oxalic acid, phytic acid, lipoic acid, vanilic acid, polyphenols, ferulic acid, theaflavins, and derivatives thereof. Those of ordinary skill in the art will appreciate other suitable antioxidants for use in the present disclosure.

In embodiments, oxygen carriers are agents characterized by the ability to carry and release oxygen. They include, without limitation, perfluorocarbons and pharmaceuticals containing perfluorocarbons. Suitable perfluorocarbon-based oxygen carriers include, without limitation, perfluorooctyl bromide (C8F17Br); perfluorodichorotane (C8F16C12); perfluorodecyl bromide; perfluobron; perfluorodecalin; perfluorotripopylamine; perfluoromethylcyclopiperidine; Fluosol® (perfluorodecalin & perfluorotripopylamine); Perftoran® (perfluorodecalin & perfluoromethylcyclopiperidine); Oxygent® (perfluorodecyl bromide & perfluobron); Ocycyte™ (perfluoro (tert-butylcyclohexane)). Those of ordinary skill in the art will appreciate other suitable perfluorocarbon-based oxygen carriers for use in the present disclosure.

Immunomodulatory factors include, without limitation, osteopontin, FAS Ligand factors, interleukins, transforming growth factor beta, platelet derived growth factor, clusterin, transferrin, regulated upon action, normal T-cell expressed, secreted protein (RANTES), plasminogen activator inhibitor-1 (Pai-1), tumor necrosis factor alpha (TNF-alpha), interleukin 6 (IL-6), alpha-1 microglobulin, and beta-2-microglobulin. Those of ordinary skill in the art will appreciate other suitable immunomodulatory factors for use in the present disclosure.

In embodiments, anti-inflammatory agents or immunosuppressant agents may also be part of the formulation. Those of ordinary skill in the art will appreciate other suitable antioxidants for use in the present formulations and/or treatments.

Cell recruitment factors include, without limitation, monocyte chemotatic protein 1 (MCP-1), and CXCL-1. Those of ordinary skill in the art will appreciate other suitable cell recruitment factors for use in the present formulations and/or treatments.

Cell attachment factors include, without limitation, fibronectin, procollagen, collagen, ICAM-1, connective tissue growth factor, laminins, proteoglycans, specific cell adhesion peptides such as RGD and YSIGR. Those of ordinary skill in the art will appreciate other suitable cell attachment factors for use in the present formulations and/or treatments.

Angiogenic factors include, without limitation, vascular endothelial growth factor F (VEGF) and angiopoietin-2 (ANG-2). Those of ordinary skill in the art will appreciate other suitable angiogenic factors for use in certain embodiments of the present disclosure.

Matrix metalloproteases include, without limitation, matrix metalloprotease 1 (MMP1), matrix metalloprotease 2 (MMP2), matrix metalloprotease 9 (MMP-9), and tissue inhibitor and matalloproteases-1 (TIMP-1).

Wound healing factors include, without limitation, keratinocyte growth factor 1 (KGF-1), tissue plasminogen activator (tPA), calbindin, clusterin, cystatin C, trefoil factor 3. Those of ordinary skill in the art will appreciate other suitable wound healing factors for use in the present formulations and/or treatments.

The disclosure also provides bioactive cell formulations containing implantable constructs comprising a biomaterial and bioactive renal cells for the treatment of kidney disease. In embodiments, the construct is made up of a biocompatible material or biomaterial, scaffold or matrix composed of one or more synthetic or naturally-occurring biocompatible materials and one or more cell populations described herein deposited on or embedded in a surface of the scaffold by attachment and/or entrapment. In embodiments, the construct is made up of a biomaterial and one or more cell populations described herein coated with, deposited on, deposited in, attached to, entrapped in, embedded in, seeded, or combined with the biomaterial component(s). Any of the cell populations described herein, including enriched cell populations (e.g., SRCs), may be used in combination with a matrix to form a construct. In embodiments, the bioactive cell formulation is made up of a biocompatible material or biomaterial and an SRC population described herein.

In embodiments, the bioactive cell formulation is a Neo-Kidney Augment (NKA), which is an injectable product composed of autologous, homologous selected renal cells (SRC) formulated in a Biomaterial (gelatin-based hydrogel). In embodiments, autologous, homologous SRC are obtained from isolation and expansion of renal cells from the patient's renal cortical tissue via a kidney biopsy and selection by separation of the expanded renal cells across a density boundary, barrier, or interface (e.g., single-step discontinuous density gradient separation). In embodiments, autologous SRC are obtained from isolation and expansion of renal cells from the patient's renal cortical tissue via a kidney biopsy and selection of the expanded renal cells over a continuous or discontinuous single step or multistep density gradient. In embodiments, the SRC are composed primarily of renal epithelial cells which are well known for their regenerative potential (Humphreys et al. (2008) Intrinsic epithelial cells repair the kidney after injury. Cell Stem Cell. 2(3):284-91). In embodiments, injection of SRC into recipient kidneys results in significant improvement in animal survival, urine concentration, and filtration functions. In embodiments, SRC have limited shelf life and stability. In embodiments, formulation of SRC in a gelatin-based hydrogel biomaterial provides enhanced stability of the cells thus extending product shelf life, improved stability of NKA during transport and delivery of NKA into the kidney cortex for clinical utility.

In embodiments, NKA is manufactured by first obtaining renal cortical tissue from a donor using a standard-of-clinical-care kidney biopsy procedure. In embodiments, the donor is the subject to be treated. In embodiments, renal cells are isolated from the kidney tissue by enzymatic digestion and expanded using standard cell culture techniques. In embodiments, a cell culture medium used expand primary renal cells does not contain any differentiation factors. In embodiments, harvested renal cells are subjected to separation across a density boundary or interface or density gradient separation to obtain SRC.

In embodiments, a formulation comprises biomaterials designed or adapted to respond to external conditions as described herein. As a result, the nature of the association of the bioactive cell population with the biomaterial in a construct changes depending upon the external conditions. In embodiments, a cell population's association with a temperature-sensitive biomaterial varies with temperature. In embodiments, the construct contains a bioactive renal cell population and biomaterial having a substantially solid state at about 8° C. or lower and a substantially liquid state at about ambient temperature or above, wherein the cell population is suspended in the biomaterial at about 8° C. or lower. In embodiments, the cell population is substantially free to move throughout the volume of the biomaterial at about ambient temperature or above. In embodiments, having the cell population suspended in the substantially solid phase at a lower temperature provides stability advantages for the cells, such as for anchorage-dependent cells, as compared to cells in a fluid. In embodiments, having cells suspended in the substantially solid state provides one or more of the following benefits: i) prevents settling of the cells, ii) allows the cells to remain anchored to the biomaterial in a suspended state; iii) allows the cells to remain more uniformly dispersed throughout the volume of the biomaterial; iv) prevents the formation of cell aggregates; and v) provides better protection for the cells during storage and transportation of the formulation. A formulation that can retain such features leading up to the administration to a subject is advantageous at least because the overall health of the cells in the formulation will be better and a more uniform and consistent dosage of cells will be administered.

In embodiments, the manufacturing process for the bioactive cell formulations is designed to deliver a product in approximately four weeks from patient biopsy to product implant. In embodiments, patient-to-patient tissue variability poses a challenge to deliver product on a fixed implant schedule. In embodiments, expanded renal cells are cryopreserved during cell expansion to accommodate for this patient-dependent variation in cell expansion. In embodiments, cryopreserved renal cells provide a continuing source of cells in the event that another treatment is needed (e.g., delay due to patient sickness, unforeseen process events, etc.) and to manufacture multiple doses for re-implantation, as required.

In embodiments, the bioactive cell composition is composed of autologous, homologous cells formulated in a biomaterial (gelatin-based hydrogel). In embodiments, the composition comprises about 20×10⁶ cells per mL to about 200×10⁶ cells per mL in a gelatin solution with Dulbecco's Phosphate Buffered Saline (DPBS). In embodiments, the number of cells per mL of product is about 20×10⁶ cells per mL, about 40×10⁶ cells per mL, about 60×10⁶ cells per mL, about 100×10⁶ cells per mL, about 120×10⁶ cells per mL, about 140×10⁶ cells per mL, about 160×10⁶ cells per mL, about 180×10⁶ cells per mL, or about 200×10⁶ cells per mL. In embodiments, the gelatin is present at about 0.5%, about 0.55%, about 0.6%, about 0.65%, about 0.7%, about 0.75%, about 0.8%, about 0.85%, about 0.9%, about 0.95% or about 1%, (w/v) in the solution. In embodiments, the biomaterial is a 0.88% (w/v) gelatin solution in DPBS. In embodiments, the injectable formulation comprises a biomaterial comprising about 0.88% (w/v) gelatin, and a composition comprising a bioactive renal cell population (BRC), wherein the BRC comprise an enriched population of tubular renal cells and having a density greater than about 1.04 g/mL. In embodiments, the injectable formulation comprises a biomaterial comprising about 0.88% (w/v) gelatin, and a composition comprising a bioactive renal cell population (BRC), wherein the BRC comprise an enriched population of tubular renal cells and having a density greater than about 1.0419 g/mL or about 1.045 g/mL.

In embodiments, NKA is presented in a sterile, single-use 10 mL syringe. In embodiments, the final volume is calculated from the concentration of 100×10⁶ SRC/mL of NKA and a target dose of 3.0×10⁶ SRC/g kidney weight. In embodiments, the kidney weight is the weight estimated by MRI. In embodiments, therapeutic dosage is determined (e.g., by a medical professional such as surgeon) at the time of injection based on the patient's kidney weight. In embodiments, the dose is about 2.5×10⁶ SRC/g kidney weight to about of 3.5×10⁶ SRC/g kidney weight.

In embodiments, a total number of cells may be selected for the formulation and the volume of the formulation may be adjusted to reach the proper therapeutic dosage. In embodiments, the formulation may contain a dosage of cells to a subject that is a single dosage or a single dosage plus additional dosages. In embodiments, the dosages may be provided by way of a construct as described herein. In embodiments, a therapeutically effective amount of a bioactive renal cell population described herein can range from the maximum number of cells that is safely received by the subject to the minimum number of cells necessary for treatment of kidney disease, e.g., stabilization, reduced rate-of-decline, or improvement of one or more kidney functions.

In embodiments, a therapeutically effective amount of a bioactive renal cell population described herein can be suspended in a pharmaceutically acceptable carrier or excipient. Such a carrier includes, but is not limited to basal culture medium plus 1% serum albumin, saline, buffered saline, dextrose, water, collagen, alginate, hyaluronic acid, fibrin glue, polyethyleneglycol, polyvinylalcohol, carboxymethylcellulose and combinations thereof. The formulation should suit the mode of administration.

In embodiments, a bioactive renal cell preparation or composition is formulated in accordance with routine procedures as a pharmaceutical composition adapted for administration to human beings. In embodiments, compositions for intravenous administration, intra-arterial administration or administration within the kidney capsule, for example, are solutions in sterile isotonic aqueous buffer. In embodiments, the composition can also include a local anesthetic to ameliorate any pain at the site of the injection. In embodiments, the ingredients are supplied either separately or mixed together in unit dosage form, for example, as a cryopreserved concentrate in a hermetically sealed container such as an ampoule indicating the quantity of active agent. In embodiments, when the composition is to be administered by infusion, it can be dispensed with an infusion bottle containing sterile pharmaceutical grade water or saline. In embodiments, where the composition is administered by injection, an ampoule of sterile water for injection or saline can be provided so that the ingredients can be mixed prior to administration.

In embodiments, pharmaceutically acceptable carriers may be determined in part by the particular composition being administered, as well as by the particular method used to administer the composition. Accordingly, there are a wide variety of suitable formulations of pharmaceutical compositions (see, e.g., Alfonso R Gennaro (ed), Remington: The Science and Practice of Pharmacy, formerly Remington's Pharmaceutical Sciences 20th ed., Lippincott, Williams & Wilkins, 2003, incorporated herein by reference in its entirety). In embodiments, the pharmaceutical compositions are generally formulated as sterile, substantially isotonic and in full compliance with all Good Manufacturing Practice (GMP) regulations of the U.S. Food and Drug Administration.

In embodiments, the bioactive cell formulation includes a cell viability agent selected from the group consisting of an antioxidant, an oxygen carrier, an immunomodulatory factor, a cell recruitment factor, a cell attachment factor, an anti-inflammatory agent, an angiogenic factor, a wound healing factor, and products secreted from bioactive cells.

In embodiments, secreted products from bioactive cells described herein may also be added to the bioactive cell formulation as a cell viability agent.

In embodiments, the formulation includes a temperature-sensitive biomaterial described herein and a population of biocompatible beads containing a biomaterial. In embodiments, the beads are crosslinked. Crosslinking may be achieved using any suitable crosslinking agent known to those of ordinary skill in the art, such as, for example, carbodiimides; aldehydes (e.g. furfural, acrolein, formaldehyde, glutaraldehyde, glyceryl aldehyde), succinimide-based crosslinkers {Bis(sulfosuccinimidyl) suberate (BS3), Disuccinimidyl glutarate (DSG), Disuccinimidyl suberate (DSS), Dithiobis(succinimidyl propionate), Ethylene glycolbis(sulfosuccinimidylsuccinate), Ethylene glycolbis(succinimidylsuccinate) (EGS), Bis(Sulfosuccinimidyl) glutarate (BS2G), Disuccinimidyl tartrate (DST)); epoxides (Ethylene glycol diglycidyl ether, 1,4 Butanediol diglycidyl ether); saccharides (glucose and aldose sugars); sulfonic acids and p-toluene sulfonic acid; carbonyldlimidazole; genipin; imines; ketones; diphenylphosphorylazide (DDPA); terephthaloyl chloride; cerium (III) nitrate hexahydrate; microbial transglutaminase; and hydrogen peroxide. Those of ordinary skill in the art will appreciate other suitable crosslinking agents and crosslinking methods for use in the present methods, formulations and/or treatments.

In embodiments, the beads are carbodlimide-crosslinked beads. In embodiments, the carbodiimide-crosslinked beads may be crosslinked with a carbodiimide selected from the group consisting of 1-Ethyl-3-[3-dimethylaminopropyl] carbodiimide hydrochloride (EDC), DCC-N,N′-dicyclohexylcarbodiimide (DCC), and N,N′-Diisopropylcarbodiimide (DIPC).

In embodiments, crosslinked beads have a reduced susceptibility to enzymatic degradation as compared to non-crosslinked biocompatible beads, thereby providing beads with finely tunable in vivo residence times. In embodiments, the crosslinked beads are resistant to endogenous enzymes, such as collagenases. In embodiments, the provision of crosslinked beads is part of a delivery system that facilitates one or more of: (a) delivery of attached cells to the desired sites and creation of space for regeneration and ingrowth of native tissue and vascular supply; (b) ability to persist at the site long enough to allow cells to establish, function, remodel their microenvironment and secrete their own extracellular matrix (ECM); (c) promotion of integration of the transplanted cells with the surrounding tissue; (d) ability to implant cells in a substantially solid form; (e) short term structural integrity that does not provide a significant barrier to tissue ingrowth or integration of delivered cells/materials with the host tissue; (f) localized in vivo delivery in a substantially solid form thereby preventing dispersion of cells within the tissue during implantation; (g) improved stability and viability of anchorage dependent cells compared to cells suspended in a fluid; and (h) biphasic release profile when cells are delivered i) in a substantially solid form (e.g., attached to beads), and ii) in a substantially liquid form (e.g., suspended in a fluid).

In embodiments, the present disclosure provides crosslinked beads containing gelatin. In embodiments, non-crosslinked gelatin beads are not suitable for a bioactive cell formulation because they rapidly lose integrity and cells dissipate from the injection site. In embodiments, highly crosslinked gelatin beads may persist too long at the injection site and may hinder the de-novo ECM secretion, cell integration and tissue regeneration. In embodiments, the present disclosure allows for the in vivo residence time of the crosslinked beads to be finely tuned. In embodiments, in order to tailor the biodegradability of biomaterials, different crosslinker concentrations of carbodiimide are used while the overall reaction conditions were kept constant for all samples. In embodiments, the enzymatic susceptibility of carbodiimide-crosslinked beads can be finely tuned by varying the concentration of crosslinking agent from about zero to about 1M. In embodiments, the concentration is about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, about 30 mM, about 31 mM, about 32 mM, about 33 mM, about 34 mM, about 35 mM, about 36 mM, about 37 mM, about 38 mM, about 39 mM, about 40 mM, about 41 mM, about 42 mM, about 43 mM, about 44 mM, about 45 mM, about 46 mM, about 47 mM, about 48 mM, about 49 mM, about 50 mM, about 55 mM, about 60 mM, about 65 mM, about 70 mM, about 75 mM, about 80 mM, about 85 mM, about 90 mM, about 95 mM, or about 100 mM. The crosslinker concentration may also be about 0.15 M, about 0.2 M, about 0.25 M, about 0.3 M, about 0.35 M, about 0.4 M, about 0.45 M, about 0.5 M, about 0.55 M, about 0.6 M, about 0.65 M, about 0.7 M, about 0.75 M, about 0.8 M, about 0.85 M, about 0.9 M, about 0.95 M, or about 1 M. In another embodiment, the crosslinking agent is 1-Ethyl-3-[3-dimethylaminopropyl] carbodiimide hydrochloride (EDC). In embodiments, the EDC-crosslinked beads are gelatin beads.

In embodiments, crosslinked beads may have certain characteristics that favor the seeding, attachment, or encapsulation. In embodiments, the beads may have a porous surface and/or may be substantially hollow. In embodiments, the presence of pores provides an increased cell attachment surface allowing for a greater number of cells to attach as compared to a non-porous or smooth surface. In embodiments, the pore structure can support host tissue integration with the porous beads supporting the formation of de novo tissue. In embodiments, the beads have a size distribution that can be fitted to a Weibull plot corresponding to the general particle distribution pattern. In embodiments, the crosslinked beads have an average diameter of less than about 120 μm, about 115 μm, about 110 μm, about 109 μm, about 108 μm, about 107 μm, about 106 μm, about 105 μm, about 104 μm, about 103 μm, about 102 μm, about 101 μm, about 100 μm, about 99 μm, about 98 μm, about 97 μm, about 96 μm, about 95 μm, about 94 μm, about 93 μm, about 92 μm, about 91 μm, or about 90 μm. In embodiments, the characteristics of the crosslinked beads vary depending upon the casting process. In embodiments, a process in which a stream of air is used to aerosolize a liquid gelatin solution and spray it into liquid nitrogen with a thin layer chromatography reagent sprayer (ACE Glassware) is used to provide beads having the aforementioned characteristics. Those of skill in the art will appreciate that modulating the parameters of the casting process provides the opportunity to tailor different characteristics of the beads, e.g., different size distributions.

In embodiments, the cytocompatibility of the crosslinked beads is assessed in vitro prior to formulation using cell culture techniques in which beads are cultured with cells that correspond to the final bioactive cell formulation. In embodiments, the beads are cultured with primary renal cells prior to preparation of a bioactive renal cell formulation and live/dead cell assays are used to confirm cytocompatibility. In embodiments, the biocompatible crosslinked beads are combined with a temperature-sensitive biomaterial in solution at about 5% (w/w) to about 15% (w/w) of the volume of the solution. In embodiments, the crosslinked beads may be present at about 5% (w/w), about 5.5% (w/w), about 6% (w/w), about 6.5% (w/w), about 7% (w/w), about 7.5% (w/w), about 8% (w/w), about 8.5% (w/w), about 9% (w/w), about 9.5% (w/w), about 10% (w/w), about 10.5% (w/w), about 11% (w/w), about 11.5% (w/w), about 12% (w/w), about 12.5% (w/w), about 13% (w/w), about 13.5% (w/w), about 14% (w/w), about 14.5% (w/w), or about 15% (w/w) of the volume of the solution.

In embodiments, the present disclosure provides formulations that contain biomaterials which degrade over a period time on the order of minutes, hours, or days. This is in contrast to a large body of work focusing on the implantation of solid materials that then slowly degrade over days, weeks, or months. In embodiments, the biomaterial has one or more of the following characteristics: biocompatibility, biodegradeability/bioresorbablity, a substantially solid state prior to and during implantation into a subject, loss of structural integrity (substantially solid state) after implantation, and cytocompatible environment to support cellular viability and proliferation. The biomaterial's ability to keep implanted particles spaced out during implantation enhances native tissue ingrowth. The biomaterial also facilitates implantation of solid formulations. The biomaterial provides for localization of the formulation described herein since insertion of a solid unit helps prevent the delivered materials from dispersing within the tissue during implantation. For cell-based formulations, a solid biomaterial also improves stability and viability of anchorage dependent cells compared to cells suspended in a fluid. However, the short duration of the structural integrity means that soon after implantation, the biomaterial does not provide a significant barrier to tissue ingrowth or integration of the delivered cells/materials with host tissue.

In an aspect, the present disclosure provides formulations that contain biomaterials which are implanted in a substantially solid form and then liquefy/melt or otherwise lose structural integrity following implantation into the body. This is in contrast to the significant body of work focusing on the use of materials that can be injected as a liquid, which then solidify in the body.

In embodiments, the present disclosure provides formulations having biocompatible crosslinked beads seeded with bioactive cells together with a delivery matrix. In embodiments, the delivery matrix has one or more of the following characteristics: biocompatibility, biodegradable/bioresorbable, a substantially solid state prior to and during implantation into a subject, loss of structural integrity (substantially solid state) after implantation, and cytocompatible environment to support cellular viability. In embodiments, the delivery matrix's ability to keep implanted particles (e.g., crosslinked beads) spaced out during implantation enhances native tissue ingrowth. In embodiments, if the delivery matrix is absent, then compaction of cellularized beads during implantation can lead to inadequate room for sufficient tissue ingrowth. In embodiments, the delivery matrix facilitates implantation of solid formulations. In embodiments, in addition, the short duration of the structural integrity means that soon after implantation, the matrix does not provide a significant barrier to tissue ingrowth or integration of the delivered cells/materials with host tissue. In embodiments, the delivery matrix provides for localization of the formulation described herein since inserted of a solid unit helps prevent the delivered materials from dispersing within the tissue during implantation. In embodiments, for cell-based formulations, a solid delivery matrix improves stability and viability of anchorage dependent cells compared to cells suspended in a fluid.

In embodiments, the delivery matrix is a population of biocompatible beads that is not seeded with cells. In embodiments, the unseeded beads are dispersed throughout and in between the individual cell-seeded beads. In embodiments, the unseeded beads act as “spacer beads” between the cell-seeded beads prior to and immediately after transplantation. In embodiments, the spacer beads contain a temperature-sensitive biomaterial having a substantially solid state at a first temperature and a substantially liquid state at a second temperature, wherein the first temperature is lower than the second temperature. In embodiments, the spacer beads contain a biomaterial having a substantially solid state at about ambient temperature or below and a substantially liquid state at about 37° C., such as that described herein. In embodiments, the ambient temperature is about room temperature. In embodiments, the biomaterial is a gelatin solution. In embodiments, the gelatin solution is present at about 4%, about 4.5%, about 5%, about 5.5%, about 6%, about 6.5%, about 7%, about 7.5%, about 8%, about 8.5%, about 9%, about 9.5%, about 10%, about 10.5%, or about 11%, (w/v). In embodiments, the gelatin solution may be provided in PBS, cell culture media (e.g., DMEM), or another suitable solvent.

In embodiments, the present disclosure provides formulations that contain biomaterials which are implanted in a substantially solid form (e.g., spacer beads) and then liquefy/melt or otherwise lose structural integrity following implantation into the body.

In embodiments, the temperature-sensitivity of spacer beads can be assessed in vitro prior to formulation. In embodiments, spacer beads can be labeled and mixed with unlabeled non-temperature-sensitive beads. In embodiments, the mixture is then Incubated at 37° C. to observe changes in physical transition. In embodiments, the loss of shape of the labeled temperature-sensitive beads at the higher temperature is observed over time. In embodiments, temperature-sensitive gelatin beads may be made with Alcian blue dye to serve as a marker of physical transition. In embodiments, the blue gelatin beads are mixed with Cultispher S beads (white), loaded into a catheter, then extruded and incubated in 1×PBS, pH 7.4, at 37° C. In embodiments, the loss of shape of the blue gelatin beads is followed microscopically at different time points. In embodiments, changes in the physical state of the blue gelatin beads are visible after 30 min becoming more pronounced with prolonged incubation times. In embodiments, the beads do not completely dissipate because of the viscosity of the material.

In embodiments, the bioactive cell formulations described herein may be used to prepare renal cell-based formulations for injection into the kidney. However, those of ordinary skill in the art will appreciate that the formulations will be suitable for many other types of bioactive cell populations. For example, the present disclosure contemplates formulations for bioactive cells for injection into any solid organ or tissue.

In embodiments, the bioactive cell formulations described herein will contain a set number of cells. In embodiments, the total number of cells for the formulation is about 10⁴, about 10⁵, about 10⁶, about 10⁷, about 10⁸, or about 10⁹. In embodiments, the dosage of cells for a formulation described herein may be calculated based on the estimated mass or functional mass of the target organ or tissue. In embodiments, the bioactive cell formulations contain a dosage corresponding to a number of cells based upon the weight of the host organ that will be the subject of treatment by the formulation. In embodiments, a bioactive renal cell formulation is based upon an average weight of about 150 grams for a human kidney. In embodiments, the number of cells per gram (g) of kidney is about 600 cells/g to about 7.0×10⁷ cells/g. In embodiments, the number of cells per gram of kidney is about 600 cells/g, about 1000 cells/g, about 1500 cells/g, about 2000 cells/g, about 2500 cells/g, about 3000 cells/g, about 3500 cells/g, about 4000 cells/g, about 4500 cells/g, about 5000 cells/g, about 5500 cells/g, about 6000 cells/g, about 6500 cells/g, about 7000 cells/g, about 7500 cells/g, about 8000 cells/g, about 8500 cells/g, about 9000 cells/g, about 9500 cells/g, or about 10,000 cells/g.

In embodiments, the number of cells per gram of kidney is about 1.5×10⁴ cells/g, about 2.0×10⁴ cells/g, about 2.5×10⁴ cells/g, about 3.0×10⁴ cells/g, about 3.5×10⁴ cells/g, about 4.0×10⁴ cells/g, about 4.5×10⁴ cells/g, about 5.0×10⁴ cells/g, about 5.5×10⁴ cells/g, about 6.0×10⁴ cells/g, about 6.5×10⁴ cells/g, about 7.0×10⁴ cells/g, about 7.5×10⁴ cells/g, about 8.0×10⁴ cells/g, about 9.5×10⁴ cells/g.

In embodiments, the number of cells per gram of kidney is about 1.0×10⁵ cells/g, about 1.5×10⁵ cells/g, about 2.0×10⁵ cells/g, about 2.5×10⁵ cells/g, about 3.0×10⁵ cells/g, about 3.5×10⁵ cells/g, about 4.0×10⁵ cells/g, about 4.5×10⁵ cells/g, about 5.0×10⁵ cells/g, about 5.5×10⁵ cells/g, about 6.0×10⁵ cells/g, about 6.5×10⁵ cells/g, about 7.0×10⁵ cells/g, about 7.5×10⁵ cells/g, about 8.0×10⁵ cells/g, about 8.5×10⁵ cells/g, about 9.0×10⁵ cells/g, or about 9.5×10⁵ cells/g.

In embodiments, the number of cells per gram of kidney is about 1.0×10⁶ cells/g, about 1.5×10⁶ cells/g, about 2.0×10⁶ cells/g, about 2.5×10⁶ cells/g, about 3.0×10⁶ cells/g, about 3.5×10⁶ cells/g, about 4.0×10⁶ cells/g, about 4.5×10⁶ cells/g, about 5.0×10⁶ cells/g, about 5.5×10⁶ cells/g, about 6.0×10⁶ cells/g, about 6.5×10⁶ cells/g, about 7.0×10⁶ cells/g, about 7.5×10⁶ cells/g, about 8.0×10⁶ cells/g about 8.5×10⁶ cells/g, about 9.0×10⁶ cells/g, about 9.5×10⁶ cells/g, 1.0×10⁷ cells/g, or about 1.5×10⁷ cells/g.

In embodiments, a total number of cells may be selected for the formulation and the volume of the formulation may be adjusted to reach the proper dosage.

In embodiments, the formulation may contain a dosage of cells to a subject that is a single dosage or a single dosage plus additional dosages. In embodiments, the dosages may be provided by way of a construct as described herein. In embodiments, the therapeutically effective amount of the renal cell populations described herein can range from the maximum number of cells that is safely received by the subject to the minimum number of cells necessary for treatment of kidney disease, e.g., stabilization, reduced rate-of-decline, or improvement of one or more kidney functions.

In embodiments, the therapeutically effective amount of the renal cell populations described herein can be suspended in a pharmaceutically acceptable carrier or excipient. Such a carriers include, but are not limited to basal culture medium plus 1% serum albumin, saline, buffered saline, dextrose, water, collagen, alginate, hyaluronic acid, fibrin glue, polyethyleneglycol, polyvinylalcohol, carboxymethylcellulose and combinations thereof. The formulation should suit the mode of administration.

In embodiments, the disclosure provides a use of a formulation containing a renal cell population for the manufacture of a medicament to treat kidney disease in a subject. In embodiments, the medicament further comprises recombinant polypeptides, such as growth factors, chemokines or cytokines. In embodiments, the medicaments comprise a human kidney-derived cell population. In embodiments, the cells used to manufacture the medicaments can be isolated, derived, or enriched using any of the variations provided for the methods described herein.

In embodiments, the renal cell preparation(s) or compositions disclosed herein are formulated in accordance with routine procedures as a pharmaceutical composition adapted for administration to human beings. In embodiments, compositions for intravenous administration, intra-arterial administration or administration within the kidney capsule, for example, are solutions in sterile isotonic aqueous buffer. In embodiments, the composition can also include a local anesthetic to ameliorate any pain at the site of the injection. In embodiments, the ingredients are supplied either separately or mixed together in unit dosage form, for example, as a cryopreserved concentrate in a hermetically sealed container such as an ampoule indicating the quantity of active agent. In embodiments, when the composition is to be administered by infusion, it can be dispensed with an infusion bottle containing sterile pharmaceutical grade water or saline. In embodiments, where the composition is administered by injection, an ampoule of sterile water for injection or saline can be provided so that the ingredients can be mixed prior to administration.

In embodiments, pharmaceutically acceptable carriers are determined in part by the particular composition being administered, as well as by the particular method used to administer the composition. Accordingly, there are a wide variety of suitable formulations of pharmaceutical compositions (see, e.g., Alfonso R Gennaro (ed), Remington: The Science and Practice of Pharmacy, formerly Remington's Pharmaceutical Sciences 20th ed., Uppincott, Williams & Wilkins, 2003, incorporated herein by reference in its entirety). In embodiments, the pharmaceutical compositions are generally formulated as sterile, substantially isotonic and in full compliance with all Good Manufacturing Practice (GMP) regulations of the U.S. Food and Drug Administration.

In embodiments, a formulation of the present disclosure is provided as a modified release formulation. In embodiments, the modified release is characterized by an initial release of a first active agent upon administration following by at least one additional, subsequent release of a second active agent. In embodiments, the first and second active agents may be the same or they may be different. In embodiments, the formulations provide modified release through multiple components in the same formulation. In embodiments, the modified release formulation contains an active agent as part of a first component that allows the active agent to move freely throughout the volume of the formulation, thereby permitting immediate release at the target site upon administration. In embodiments, the first component may be a temperature-sensitive biomaterial having a substantially liquid phase and a substantially solid phase, wherein the first component is in a substantially liquid phase at the time of administration. In embodiments, the active agent in the substantially liquid phase such that it is substantially free to move throughout the volume of the formulation, and therefore is immediately released to the target site upon administration.

In embodiments, the modified release formulation has an active agent as part of a second component in which the active agent is attached to, deposited on, coated with, embedded in, seeded upon, or entrapped in the second component, which persists before and after administration to the target site. In embodiments, the second component contains structural elements with which the active agent is able to associate with, thereby preventing immediate release of the active agent from the second component at the time of administration. In embodiments, the second component is provided in a substantially solid form, e.g., biocompatible beads, which may be crosslinked to prevent or delay in vivo enzymatic degradation. In embodiments, the active agent in the substantially solid phase retains its structural integrity within the formulation before and after administration and therefore it does not immediately release the active agent to the target site upon administration. Suitable carriers for modified release formulations have been described herein but those of ordinary skill in the art will appreciate other carriers that are appropriate for use herein.

In embodiments, the formulation provides an initial rapid delivery/release of delivered elements, including cells, nanoparticles, therapeutic molecules, etc. followed by a later delayed release of elements. In embodiments, the formulations of the present disclosure can be designed for such biphasic release profile where the agent to be delivered is provided in both an unattached form (e.g., cells in a solution) and an attached form (e.g., cells together with beads or another suitable carrier). In embodiments, upon initial administration, the unencumbered agent is provided immediately to the site of delivery while release of the encumbered agent is delayed until structural integrity of the carrier (e.g., beads) fails at which point the previously attached agent is released. As discussed herein, other suitable mechanisms of release will be appreciated by those of ordinary skill in the art.

In embodiments, the time delay for release can be adjusted based upon the nature of the active agent. In embodiments, the time delay for release in a bioactive cell formulation may be on the order of seconds, minutes, hours, or days. In embodiments, a delay on the order of weeks may be appropriate. In embodiments, for other active agents, such as small or large molecules, the time delay for release in a formulation may be on the order of seconds, minutes, hours, days, weeks, or months. In embodiments, it is also possible for the formulation to contain different biomaterials that provide different time delay release profiles. In embodiments, a first biomaterial with a first active agent may have a first release time and a second biomaterial with a second active agent may have a second release time. In embodiments, the first and second active agent may be the same or different.

In embodiments, the time period of delayed release may generally correspond to the time period for loss of structural integrity of a biomaterial. However, those of ordinary skill in the art will appreciate other mechanisms of delayed release. In embodiments, an active agent may be continually released over time independent of the degradation time of any particular biomaterial, e.g., diffusion of a drug from a polymeric matrix. In embodiments, bioactive cells can migrate away from a formulation containing a biomaterial and the bioactive cells to native tissue. In embodiments, bioactive cells migrate off of a biomaterial, e.g., a bead, to the native tissue.

In embodiments, biodegradable, biocompatible polymers can be used, such as ethylene vinyl acetate, polyanhydrides, polyglycolic acid, collagen, polyorthoesters, and polylactic acid. In embodiments, prolonged absorption of injectable formulations can be brought about by including in the formulation an agent that delays absorption, for example, monostearate salts and gelatin. Many non-limiting examples of methods for the preparation of such formulations are patented or generally known to those skilled in the art. See, e.g., Sustained and Controlled Release Drug Delivery Systems, J. R. Robinson, ed., Marcel Dekker, Inc., New York, 1978. Additional non-limiting examples of methods applicable to the controlled or extended release of polypeptide agents are described, for example, in U.S. Pat. Nos. 6,306,406 and 6,346,274, as well as, for example, in U.S. Patent Application Nos. US20020182254 and US20020051808, all of which are incorporated herein by reference.

In embodiments, a formulation provided herein is administered alone. In embodiments, a formulation provided herein is administered in combination with one or more other active compositions. In embodiments, a formulation is suitable for injection or implantation of incorporated tissue engineering elements to the interior of a solid organ to regenerate tissue. In embodiments, the formulations are used for the injection or implantation of tissue engineering elements to the wall of a hollow organ to regenerate tissue.

Also provided by the disclosure herein are methods of providing a bioactive cell formulation to a subject. In embodiments, the source of the bioactive cell may be autologous, allogeneic, syngeneic (autogeneic or isogeneic), and any combination thereof. In embodiments, in instances where the source is not autologous, the methods may include the administration of an immunosuppressant agent. (see e.g. U.S. Pat. No. 7,563,822). Examples of immunosuppressant drugs include, without limitation, azathioprine, cyclophosphamide, mizoribine, ciclosporin, tacrolimus hydrate, chlorambucil, Iobenzarit disodium, auranofin, alprostadil, gusperimus hydrochloride, biosynsorb, muromonab, alefacept, pentostatin, daclizumab, sirolimus, mycophenolate mofetil, leflonomide, basiliximab, dornase a, bindarid, cladribine, pimecrolimus, ilodecakin, cedelizumab, efalizumab, everolimus, anisperimus, gavilimomab, faralimomab, clofarabine, rapamycin, siplizumab, saireito, LDP-03, CD4, SR-43551, SK&F-106615, IDEC-114, IDEC-131, FTY-720, TSK-204, LF-080299, A-86281, A-802715, GVH-313, HMR-1279, ZD-7349, IPL-423323, CBP-1011, MT-1345, CNI-1493, CBP-2011, J-695, UP-920, L-732531, ABX-RB2, AP-1903, IDPS, BMS-205820, BMS-224818, CTLA4-1g, ER-49890, ER-38925, ISAtx-247, RDP-58, PNU-156804, UP-1082, TMC-95A, TV-4710, PTR-262-MG, and AGI-1096 (see U.S. Pat. No. 7,563,822). Those of ordinary skill in the art will appreciate other suitable immunosuppressant drugs.

In embodiments, at least one active agent (such as a renal cell population, a product thereof, or a spheroid comprising a renal cell population and one or more non-renal cell types or populations) is directly administered to the site of intended benefit, e.g., by injection. In embodiments, a subject may be treated by in vivo contacting of a native kidney with a bioactive cell formulation described herein together with products secreted from one or more enriched renal cell populations, and/or a mixture or construct containing the same. In embodiments, the step of in vivo contacting provides a regenerative effect to the native kidney.

A variety of means for administering compositions of active agents such as selected renal cells to subjects will, in view of this specification, be apparent to those of skill in the art. Such methods include injection of the cells into a target site in a subject.

Modes of administration of the formulations include, but are not limited to, systemic, intra-renal (e.g., parenchymal), intravenous or intra-arterial injection and injection directly into the tissue at the intended site of activity. Additional modes of administration to be used in accordance with certain embodiments herein include single or multiple injection(s) via direct laparotomy, via direct laparoscopy, transabdominal, or percutaneous. Still yet additional modes of administration to be used in accordance with embodiments include, for example, retrograde and ureteropelvic infusion. Surgical means of administration include one-step procedures such as, but not limited to, partial nephrectomy and construct implantation, partial nephrectomy, partial pyelectomy, vascularization with omentum±peritoneum, multifocal biopsy needle tracks, cone or pyramidal, to cylinder, and renal pole-like replacement, as well as two-step procedures including, for example, organoid-internal bioreactor for replanting. In embodiments, formulations containing different active agents are delivered via the same route at the same time. In embodiments, active agents are delivered separately to specific locations or via specific methodologies, either simultaneously or in a temporally-controlled manner, by one or more of the methods described herein. In embodiments, at least one active agent (such as a renal cell population, a product thereof, or a spheroid comprising a renal cell population and one or more non-renal cell types or populations) is percutaneously injected into the renal cortex of a kidney. In embodiments, a guiding cannula is inserted percutaneously and used to puncture the kidney capsule prior to injection of the composition into the kidney.

In embodiments, a laparoscopic or percutaneous technique may be used to access the kidney for injection of formulated BRC or SRC population. In embodiments, use of laparoscopic surgical techniques allows for direct visualization of the kidney so that any bleeding or other adverse events can be spotted during injection and addressed immediately. In embodiments, use of a percutaneous approach to the kidney has been in use for over a decade, primarily for ablating intrarenal masses. In embodiments, these procedures insert an electrode or cryogenic needle into a defined mass in the kidney, and remain in contact for (typically) 10 to 20 minutes while the lesion is ablated. In embodiments, for injection of the therapeutic formulation, the percutaneous instrumentation is no larger nor more complex, and this approach offers the safety advantages of no surgery (avoiding abdominal puncture wounds and inflation with gas) and minimal immobilization time. In embodiments, the access track can have hemostatic biodegradable material left in place, to further reduce any chance of significant bleeding.

In embodiments, the therapeutic formulation is injected into the renal cortex. In embodiments, it is important to distribute the therapeutic formulation in the renal cortex as widely as possible. In embodiments, distributing the therapeutic formulation in the renal cortex is achieved by entering the renal cortex at an angle allowing deposition of the therapeutic formulation in the renal cortex as widely as feasible. In embodiments, the kidney is imaged in a longitudinal or transverse approach using ultrasound guidance or with axial computed tomography (CT) imaging, depending upon individual patient characteristics. In embodiments, the injection will involve multiple deposits as the injection needle/cannula is gradually withdrawn. In embodiments, the full volume of the therapeutic formulation may be deposited at a single or multiple entry points. In embodiments, up to two entry points may be used to deposit the full volume of therapeutic formulation into the kidney. In embodiments, the injection may be administered to a single kidney, using one or more entry points, e.g. one or two entry points. In embodiments, the injection is made into both kidneys, in each kidney using one or more entry point, e.g. one or two entry points. In embodiments, a composition provided herein is administered to a subject multiple times over a given time period, e.g., two or more times, wherein each administration is at least about 1, 2, 3, 4, 5, 6 or 12 months after the previous administration. In embodiments, the SRCs are administered as a single treatment into one kidney. In embodiments, the BRCs (e.g., SRCs) are administered as a single treatment with injections into both kidneys. In embodiments, the BRCs (e.g., SRCs) are administered as repeated or multiple injections into one or both kidneys. In embodiments, the first and second injections are administered at least 3 months apart, at least 6 months apart, or at least one year apart. In embodiments, the BRCs (e.g., SRCs) are administered over more than 2 injections. In embodiments, the composition administered as a single injection or multiple injections over a specified time period. In embodiments, the composition is administered with a minimum of one injection in one kidney. In embodiments, the composition is administered as two or more injections. In embodiments, the first and second injections may be administered at any time up to 3 months apart, any time up to 6 months apart or at annual intervals. In embodiments, the second injection is administered any time up to 3 years after the 1^{st} injection. In embodiments, the composition may also be administered as one, two or more injections in one or both kidneys. In embodiments, the composition is administered to subjects who contemporaneously receive standard-of-care treatment for CKD prior to receiving injections of NKA. In embodiments, the two or more injections do not result in adverse immunogenic effects. In embodiments, the composition is injected into one kidney of the patient. In embodiments, the composition is injected into both kidneys of the patient. In embodiments, single or multiple entry points may be used to inject the composition into the kidney of the patient. In embodiments, the injection is into the renal parenchyma. In embodiments, the patient receives a therapeutic dose at any given injection site. In embodiments, the patient receives a dose of 1-9×10⁶ SRC/g of kidney at any given injection site.

In embodiments, the step of contacting a native kidney in vivo with secreted products may be accomplished through the use/administration of a formulation containing a population of secreted products from cell culture media, e.g., conditioned media, and/or by implantation of an enriched cell population, and/or a construct capable of secreting the products in vivo. In embodiments, the step of in vivo contacting provides a regenerative effect to the native kidney.

A variety of means for administering cells and/or secreted products to subjects will, in view of this specification, be apparent to those of skill in the art. In embodiments, such methods include injection of the cells into a target site in a subject.

In embodiments, cells and/or secreted products can be inserted into a delivery device or vehicle, which facilitates introduction by injection or implantation into the subjects. In embodiments, the delivery vehicle can include natural materials. In embodiments, the delivery vehicle can include synthetic materials. In embodiments, the delivery vehicle provides a structure to mimic or appropriately fit into the organ's architecture. In embodiments, the delivery vehicle is fluid-like in nature. In embodiments, such delivery devices can include tubes, e.g., catheters, for injecting cells and fluids into the body of a recipient subject. In embodiments, the tubes additionally have a needle, e.g., a syringe, through which the cells can be introduced into the subject at a desired location. In embodiments, mammalian kidney-derived cell populations are formulated for administration into a blood vessel via a catheter (where the term “catheter” is intended to include any of the various tube-like systems for delivery of substances to a blood vessel). In embodiments, the cells can be inserted into or onto a biomaterial or scaffold, including but not limited to textiles, such as weaves, knits, braids, meshes, and non-wovens, perforated films, sponges and foams, and beads, such as solid or porous beads, microparticles, nanoparticles, and the like (e.g., Cultispher-S gelatin beads-Sigma). In embodiments, the cells can be prepared for delivery in a variety of different forms. In embodiments, the cells can be suspended in a solution or gel. In embodiments, cells can be mixed with a pharmaceutically acceptable carrier or diluent in which the cells remain viable. In embodiments, pharmaceutically acceptable carriers and diluents include saline, aqueous buffer solutions, solvents and/or dispersion media. The use of such carriers and diluents is well known in the art. In some embodiments, the solution is a sterile fluid, and will often be isotonic. In embodiments, the solution is stable under the conditions of manufacture and storage and preserved against the contaminating action of microorganisms such as bacteria and fungi through the use of, for example, parabens, chlorobutanol, phenol, ascorbic acid, thimerosal, and the like. One of skill in the art will appreciate that the delivery vehicle used in the delivery of the cell populations and/or admixtures thereof can include combinations of the above-mentioned characteristics.

In an aspect, provided herein is a method of treating kidney disease in a subject, the method comprising injecting a formulation, composition, or cell population disclosed herein into the subject. In embodiments, the formulation, composition, for cell population is injected through a 18 to 30 gauge needle. In embodiments, the formulation, composition, for cell population is injected through a needle that is smaller than 20 gauge. In embodiments, the formulation, composition, for cell population is injected through a needle that is smaller than 21 gauge. In embodiments, the formulation, composition, for cell population is injected through a needle that is smaller than 22 gauge. In embodiments, the formulation, composition, for cell population is injected through a needle that is smaller than 23 gauge. In embodiments, the formulation, composition, for cell population is injected through a needle that is smaller than 24 gauge. In embodiments, the formulation, composition, for cell population is injected through a needle that is smaller than 25 gauge. In embodiments, the formulation, composition, for cell population is injected through a needle that is smaller than 26 gauge. In embodiments, the formulation, composition, for cell population is injected through a needle that is smaller than 27 gauge. In embodiments, the formulation, composition, for cell population is injected through a needle that is smaller than 28 gauge. In embodiments, the formulation, composition, for cell population is injected through a needle that is smaller than 29 gauge. In embodiments, the formulation, composition, for cell population is injected through a needle that is about 20 gauge. In embodiments, the formulation, composition, for cell population is injected through a needle that is about 21 gauge.

In embodiments, the formulation, composition, for cell population is injected through a needle that is about 22 gauge. In embodiments, the formulation, composition, for cell population is injected through a needle that is about 23 gauge. In embodiments, the formulation, composition, for cell population is injected through a needle that is about 24 gauge. In embodiments, the formulation, composition, for cell population is injected through a needle that is about 25 gauge. In embodiments, the formulation, composition, for cell population is injected through a needle that is about 26 gauge. In embodiments, the formulation, composition, for cell population is injected through a needle that is about 27 gauge. In embodiments, the formulation, composition, for cell population is injected through a needle that is about 28 gauge. In embodiments, the formulation, composition, for cell population is injected through a needle that is about 29 gauge.

In embodiments, the inter diameter of the needle is less than 0.84 mm. In embodiments, the inter diameter of the needle is less than 0.61 mm. In embodiments, the inter diameter of the needle is less than 0.51 mm. In embodiments, the inter diameter of the needle is less than 0.41 mm. In embodiments, the inter diameter of the needle is less than 0.33 mm. In embodiments, the inter diameter of the needle is less than 0.25 mm. In embodiments, the inter diameter of the needle is less than 0.20 mm. In embodiments, the inter diameter of the needle is less than 0.15 mm. In embodiments, the outer diameter of the needle is less than 1.27 mm. In embodiments, the outer diameter of the needle is less than 0.91 mm. In embodiments, the outer diameter of the needle is less than 0.81 mm. In embodiments, the outer diameter of the needle is less than 0.71 mm. In embodiments, the outer diameter of the needle is less than 0.64 mm. In embodiments, the outer diameter of the needle is less than 0.51 mm. In embodiments, the outer diameter of the needle is less than 0.41 mm. In embodiments, the outer diameter of the needle is less than 0.30 mm. In certain embodiments, a needle has one of the sizes in the following table:

| | ID Size | | OD Size | |
|---|---|---|---|---|
| Guage | in | mm | in | mm |
| 14 | 0.060 | 1.55 | 0.070 | 1.83 |
| 15 | 0.054 | 1.37 | 0.065 | 1.65 |
| 16 | 0.047 | 1.19 | n/a | n/a |
| 18 | 0.033 | 0.84 | 0.050 | 1.27 |
| 20 | 0.023 | 0.61 | 0.036 | 0.91 |
| 21 | 0.020 | 0.51 | 0.032 | 0.81 |
| 22 | 0.016 | 0.41 | 0.028 | 0.71 |
| 23 | 0.013 | 0.33 | 0.025 | 0.64 |
| 25 | 0.010 | 0.25 | 0.020 | 0.51 |
| 27 | 0.008 | 0.20 | 0.016 | 0.41 |
| 30 | 0.006 | 0.15 | 0.012 | 0.30 |
| 32 | 0.004 | 0.10 | 0.009 | 0.23 |

A non-limiting example of a cell-containing therapeutic product is the Neo Kidney Augment (NKA). NKA comprises SRCs (i.e., homologous, autologous selected renal cells) as a biologically active component. Without being bound by any scientific theory, this cell population is naturally involved in renal repair and regeneration. (Bruce et al. Regen Med. 2015; 10:815-39; Bruce et al. Experimental Biology Meeting, Washington, D C, 2011; Genheimer et al. Cells Tissues Organs. 2012; 196:374-84; Ilagan et al. TERMIS Conference, Orlando, Fla., 2010; Ilagan et al. TERMIS Conference, Orlando, Fla., 2010; Ilagan et al. KIDSTEM Conference, Liverpool, U K, 2009; Kelley et al. Cell Transplant. 2013; 22:1023-39; Kelley et al. ADA Conference, San Diego, Calif., 2011; Kelley et al. ISCT Conference, Philadelphia, Pa., 2010; Kelley et al. KIDSTEM Conference, Liverpool, U K, 2008; Kelley et al. TERMIS Conference, Orlando, Fla., 2010; Presnell et al. Tissue Engineering Part C Methods. 2010; 17:261-73; Presnell et al. Experimental Biology Meeting, New Orleans, L A, 2009; Wallace et al. ISCT Conference, Philadelphia, Pa., 2010; Yamaleyeva et al. TERMIS Conference, Orlando, Fla., 2010). In embodiments, therapeutic intervention with NKA improves renal function in subjects with CKD and CAKUT and delays the need for renal dialysis or transplantation which, based on the current standard-of-care, is inevitable for ESRD.

NKA is made from expanded autologous selected renal cells (SRC) obtained from each individual subject's kidney biopsy. In embodiments, to manufacture NKA, kidney biopsy tissue from a subject is processed to have renal cells expanded and SRC selected.

In embodiments, NKA is presented in a sterile, single-use syringe. In embodiments, SRC is formulated in a gelatin based hydrogel at a concentration of 100×10⁶ cells/mL, packaged in a 10 mL syringe, and shipped to the clinical site for use. In embodiments, the final volume is calculated from the concentration of 100×10⁶ SRC/mL of NKA and the target dose of 3.0×10⁶ SRC/g kidney weight (estimated by, e.g., MRI). In embodiments, as described in the literature, volume measurements of the kidney in mLs obtained by different methods are approximately 92-97% of dry weight measurements in grams obtained by measuring isolated organs trimmed of perirenal fat. In embodiments, a dose of NKA is calculated using a conversion of 1 g equals 1 mL. In embodiments, dosage is determined at the time of injection based on the patient's kidney weight. In embodiments, the maximum volume for any patient will be 8.0 mL; that is, if any subject has a left kidney with a calculated weight ≥259 g, then that subject will receive 8 mL of NKA.

Expanded renal cells can be cryopreserved during cell expansion to accommodate for patient-dependent variation in cell expansion. Cryopreserved renal cells provide a continuing source of cells to manufacture multiple doses of the bioactive cell formulation for re-injection and in the event that another treatment is needed (e.g., delay due to patient sickness, unforeseen process events, etc.).

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### EXAMPLES

### Example 1—Phase I, Open-Label Safety, Tolerability, and Early Efficacy Study of Renal Autologous Cell Therapy (REACT) in Patients with Chronic Kidney Disease from Congenital Anomalies of the Kidney and Urinary Tract (CAKUT) (REGEN-044)

### Protocol Synopsis

Therapeutic Product

REACT is made from expanded autologous selected renal cells (SRC) obtained from each individual subject's kidney biopsy. To manufacture REACT, kidney biopsy tissue from each enrolled subject is sent to Twin City Bio LLC, where renal cells are expanded and SRC selected. SRC are formulated in a gelatin based hydrogel at a concentration of 100×10⁶ cells/mL, packaged in a 10 mL syringe, and shipped to the clinical site for use.

Study Objectives

Primary Objective: The primary objective of the study is to assess the safety of REACT injected in one recipient kidney.

Primary Endpoints:
Change in eGFR through 6 months following two REACT injections
Incidence of renal-specific procedure and/or product related adverse events (AEs) through 6 months post-injection
   Secondary Objective: The secondary objective of the study is to assess the safety and tolerability of REACT administration by assessing renal-specific adverse events over a 24 month period following injection.

Secondary endpoint:
Renal-specific laboratory assessments through 24 months post-injection.
   Exploratory Objective: Exploratory objectives of the study are designed to assess the impact of REACT on renal function over a 24 month period following injection.

Exploratory endpoints:
Clinical diagnostic and laboratory assessments of renal structure and function (including eGFR, serum creatinine, and proteinuria) to assess changes in the rate of progression of renal disease.
Vitamin D levels
Iohexol imaging
Blood pressure control
MRI assessment of kidney volume

Study Design

Multi-center, prospective, open-label, single-group study. All subjects are treated with two REACT injections 3 months (+12 weeks) apart after biopsy.

Randomization

Open-label, non-randomized.

Control Group

Each subject serves as his or her own control; the patient's previous medical history, which must include a minimum 6 month period of observation of renal function, serves as the control for rate of progression of renal insufficiency.

Sample Size

Up to 15 patients are treated with REACT. As this is a Phase I safety study, robust statistical analysis is not to be performed. Therefore, the sample size proposed for this study is a size typical for in Phase 1 studies, allowing for identification of safety outcomes in a limited population.

Study Population

Male or female patients 18 to 65 years of age with CKD defined as eGFR between 14 and 50 mL/min/1.73 m² as a result of CAKUT. Patients should have sufficient historical clinical data (no fewer than three eGFR measurements) to determine their individual rate of CKD progression.

Inclusion Criteria: Unless otherwise noted, subjects must satisfy each inclusion criterion to participate in the study. Inclusion criteria is to be assessed at the Screening Visit, prior to renal biopsy, and before each REACT injection unless otherwise specified.

1. The patient is male or female, 18 to 65 years of age on the date of informed consent.
2. The patient has a documented history of abnormality of the kidney and/or urinary tract in addition to documented history of CAKUT.
3. The patient has an established diagnosis of Stage III/IV CKD not requiring renal dialysis, defined as having an eGFR between 14 and 50 mL/min/1.73 m2 inclusive at the Screening Visit prior to REACT injection.
4. The subject has blood pressure less than 140/90 at the Screening Visit, prior to renal biopsy, and prior to REACT injection(s). Note BP should not be significantly below 115/70.
5. A minimum of three measurements of eGFR or sCr should be obtained at least 3 months apart prior to the Screening Visit and within the previous 24 months to define the rate of progression of CKD.
6. The patient is willing and able to refrain from NSAID consumption (including aspirin) as well as clopidogrel, prasugrel, or other platelet inhibitors during the period beginning 7 days before through 7 days after both the renal biopsy and REACT injection(s).
7. The patient is willing and able to refrain from consumption of fish oil and platelet aggregation inhibitors, such as dipryridamole (i.e., Persantine®), during the period beginning 7 days before through 7 days after both the renal biopsy and REACT injection(s).
8. The patient is willing and able to cooperate with all aspects of the protocol.
9. The patient is willing and able to provide signed informed consent.

Exclusion Criteria: Subjects who satisfy any exclusion criterion listed below are not eligible to participate in the study. Exclusion criteria is assessed at the Screening Visit, before renal biopsy, and before each REACT injection unless otherwise noted.

1. The patient has a history of renal transplantation.
2. The patient has a diagnosis of hydronephrosis, SFU Grade 4 or 5.
3. The patient has an uncorrected VUR Grade 5.
4. The patient's cortical thickness measures less than 5 mm on MRI
5. The patient has a known allergy or contraindication(s), or has experienced severe systemic reaction(s) to kanamycin or structurally similar aminoglycoside antibiotic(s)
6. The patient has a history of anaphylactic or severe systemic reaction(s) or contraindication(s) to human blood products or materials of animal origin (e.g., bovine, porcine).
7. The patient has a history of severe systemic reaction(s) or any contraindication to local anesthetics or sedatives.
8. The patient has a clinically significant infection requiring parenteral antibiotics within
6 weeks of REACT injection.
9. The patient has acute kidney injury or has experienced a rapid decline in renal function during the last 3 months prior to REACT injection.
10. The patient has any of the following conditions prior to REACT injection: renal tumors, polycystic kidney disease, anatomic abnormalities that would interfere with the REACT injection procedure or evidence of a urinary tract infection.
Note: anatomic abnormalities are not exclusionary if kidney remains accessible and meets the criteria to receive REACT injection
11. The patient has class III or IV heart failure (NYHA Functional Classification)
12. The patient has FEV1/FVC ≥70%.
13. The patient has a history of cancer within the past 3 years (excluding non-melanoma skin cancer and carcinoma in situ of the cervix).
14. The patient has clinically significant hepatic disease (ALT or AST greater than 3 times the upper limit of normal) as assessed at the Screening Visit.
15. The patient is positive for active infection with Hepatitis B Virus (HBV), or Hepatitis C Virus (HCV), and/or Human Immunodeficiency Virus (HIV) as assessed at the Screening Visit.
16. The patient has a history of active tuberculosis (TB) requiring treatment within the past 3 years.
17. The patient is immunocompromised or is receiving immunosuppressive agents, including individuals treated for chronic glomerulonephritis within 3 months of REACT injection.
Note: inhaled corticosteroids and chronic low-dose corticosteroids (less than or equal to 7.5 mg per day) are permitted as are brief pulsed corticosteroids for intermittent symptoms (e.g., asthma).
18. The patient has a life expectancy less than 2 years.
19. The female patient is pregnant, lactating (breast feeding), or planning a pregnancy during the course of the study. Or, the female patient is of child-bearing potential and is not using a highly effective method(s) of birth control, including sexual abstinence. Or, the female patient is unwilling to continue using a highly-effective method of birth control throughout the duration of the study.
20. The patient has a history of active alcohol and/or drug abuse that, in the judgment of the Investigator, would impair the patient's ability to comply with the protocol.
21. The patient's health status would, in the judgment of the Investigator, be jeopardized by participating in the study.
22. The patient has used an investigational product within 3 months prior to REACT injection without receiving written consent from the Medical Monitor.

Study Duration

Treatment begins as soon as the REACT product is made available and assuming a one month interval prior to receiving the first REACT injection, and assuming a 3 month interval before receiving the second injection, plus a 24 month follow up period after the final injection, the duration of treatment would be:
28 months for a series of 2 REACT injections

Study Enrollment

Up to 15 subjects are enrolled into the study. Patients who complete screening procedures satisfying all I/E criteria are enrolled into the study immediately prior to the biopsy. Patients who do not meet all criteria before the biopsy is taken are considered screen failures. Patients who have a biopsy but are not injected for whatever reason are discontinued from the study and may be replaced. Once a patient has been injected, the patient completes treatment and every effort is made to ensure the patient completes all follow-up visits.

Investigational Plan

Screening: Subjects who satisfy eligibility criteria and provide written informed consent may be entered into the study. The subject should have adequate, historical clinical data to provide a reasonable estimate of the rate of progression of CKD following consultation with the Medical Monitor. Screening procedures include a full physical exam, ECG, and laboratory assessments (hematology, clinical chemistry, and urinalysis). An ultrasound is performed to confirm anatomic features of the kidney to be biopsied and injected. An MRI or Ultrasound is completed to determine kidney size and volume to determine dose volume.
Renal Biopsy: Three days or less before undergoing renal biopsy, enrolled subjects report to the clinic and undergo an interim physical exam along with an ECG and renal MRI (if not completed during or after the Screening Visit). Laboratory tests, including renal function, hemoglobin, and a pregnancy test for females also are performed. Eligible subjects satisfying all inclusions and exclusion criteria are admitted to the hospital/clinical research center to undergo a kidney biopsy. A minimum of 2 tissue cores measuring at 1.5 cm a piece must be collected using a 16 gauge biopsy needle to provide sufficient material for the manufacture of REACT. Subjects who do not experience complications from the biopsy may be discharged the same day consistent with site standard practice. Each individual subject's kidney biopsy tissue is sent to Twin City Bio LLC.
REACT Injection: Ten to 14 days before the scheduled injection date, subjects undergo an interim physical exam for ongoing verification of inclusion and exclusion criteria. Subjects also undergo renal scintigraphy (i.e., split kidney function scan) to find out what percentage each kidney contributes to total baseline kidney function. On the day of the scheduled REACT injection, eligible subjects are admitted into the hospital/clinical research unit. After warming to liquefy the hydrogel, REACT is injected into the same kidney that was previously biopsied using a percutaneous approach. This procedure will follow a standardized technique, such as that used in the ablation of renal masses by radiofrequency or cryogenic methods. Subjects without complications may be discharged the same day consistent with site standard practice. An ultrasound is performed the day after injection to detect possible, subclinical AEs. Subjects receive 2 REACT injections given 3 months (+12 weeks) apart. The first and second injections occur in the same kidney in which the biopsy was taken. Therefore, only one kidney is used for the duration of this study.
Follow-up: Subjects complete follow-up evaluations on Days 1, 7, 14, 28 (±3 days) and Month 2±7 days) after the first and second REACT injections. Depending on when the second injection is administered (i.e., at 3 months [+12 weeks]), subjects may undergo evaluations at 3 and 6 months after the first REACT injection. Following the final REACT injection, subjects complete long-term, follow-up assessments of safety and efficacy through 6, 9, 12, 15, 18, 21, and 24 months post-treatment.
Safety Monitoring: Hemorrhage following REACT injection is a known and foreseeable risk to subjects participating in this study. Therefore, hemoglobin is measured by the site's local laboratory at the following times: a) before, b) after procedure per site standard practice

### Investigational Product, Dosage and Mode of Administration

Investigational Product: REACT is made from expanded autologous selected renal cells obtained from each individual subject's kidney biopsy. To manufacture REACT, biopsy tissue from each enrolled subject is sent to Twin City Bio LLC, in whose facilities renal cells are expanded and SRC selected. SRC are formulated in a gelatin-based hydrogel at a concentration of 100×10⁶ cells/mL, packaged in a 10 mL syringe, and shipped to the clinical site.
Dosage: The volume of REACT to be administered is determined by pre-procedure MRI volumetric 3D evaluation or ellipsoid formula (Length×width AP plane×width Transverse plan×0.62). Based on pre-clinical data, the dose of REACT will be 3×10⁶ cells/g estimated kidney weight (g KW^{est}). Since the concentration of SRC per mL of REACT is 100×10⁶ cells/mL, the dosing volume will be 3.0 mL for each 100 g of kidney weight. Using this dosing paradigm, the following table shows the dosing volume and number of SRC to be delivered relative to estimated kidney weight. The maximum volume of REACT injected into the biopsied kidney will be 8.0 mL.

| Estimated | | | |
|---|---|---|---|
| Kidney Weight (gKW^{est})* | | Dosing | SRC Delivered |
| Median Weight | Weight Range | Volume | (Number |
| (g) | (g) | (mL) | of Cells × 10⁶) |
| 100 | 95-108 | 3.0 | 300 |
| 117 | 109-125 | 3.5 | 350 |
| 133 | 126-141 | 4.0 | 400 |
| 150 | 142-158 | 4.5 | 450 |
| 167 | 159-175 | 5.0 | 500 |
| 183 | 176-191 | 5.5 | 550 |
| 200 | 192-208 | 6.0 | 600 |
| 217 | 209-225 | 6.5 | 650 |
| 233 | 226-241 | 7.0 | 700 |
| 250 | 242-258 | 7.5 | 750 |
| — | >259 | 8.0 | 800 |

*Kidney weight will be estimated from the results of an MRI study performed

Subjects receive two planned REACT injections to allow dose-finding and evaluate the duration of effects. The first and second injections occur in the same kidney in which the biopsy was taken. In some cases, a subject or the Investigator may decide to postpone or withhold the second REACT injection. For example, if there appears to be any untoward safety risk, or rapid deterioration of renal function, or the development of uncontrolled diabetes or uncontrolled hypertension, or the development a malignancy or an intercurrent infection, then the second REACT injection should not be administered.
Mode of Administration: REACT is injected into the biopsied kidney using a percutaneous approach. The percutaneous method employs a standardized technique (such as that utilized in the ablation of renal masses by radiofrequency or cryogenic methods).

Statistical Analysis Methods

Statistical analyses is primarily descriptive in nature and no statistical hypothesis testing is planned for the study. Unless otherwise specified, continuous variables are summarized by presenting the number of non-missing observations (n), mean, standard deviation, median, minimum, and maximum. Categorical variables are summarized by presenting frequency count and percentage for each category.

TABLE 1
Time and Events Table

| | Screening | Renal Biopsy | | | | First REACT Injection | | | Follow-up First |
|---|---|---|---|---|---|---|---|---|---|
| | Visit | | | Day 1 | | | Day 0 | Day 1 | REACT Injection |
| | Day −60 | Day −3 | Day 0 | Follow- | | Day −14 | REACT | Follow- | Day 7 (±) |
| Clinical Assessment: | to −3ⁿ | to −1 | Biopsy | up | | to −10 | Injection | up | 3 days |
| Obtain Informed Consent^{e} | X | | | | Preparation | | | | |
| Verify I/E Criteria | X | X | X | | and Shipment | X | X | | |
| Obtain Demographic Data | X | | | | of REACT | | | | |
| Obtain Medical History | X | | | | Product* | | | | |
| Record ConMeds | X | X | X | X | | X | X | X | X |
| Perform Comprehensive PE^{f} | X | | | | | | | | |
| Perform Interm PE^{f} | | X | | | | X | | | |
| Measure Vital Signs^{g} | X | X | X | X | | X | X^{h} | X | X |
| Conduct Laboratory Testsⁱ | X | X | X | X | | X | X | X | X |
| Perform 12-lead ECG | X | X | | | | X | | | |
| Perform Ultrasound | X^{j} | | X^{k} | X^{k} | | X^{j} | X^{k} | X^{k} | |
| Perform MRI Study | →→→X^{l} | | | | | | | | |
| Perform Renal Scintigraphy | | | | | | X^{m} | | | |
| Admit to Hospital | | | X^{p} | | | | X^{p} | | |
| Perform Kidney Biopsy | | | X | | | | | | |
| Monitor/Record AEs | | X | X | X | | X | X | X | X |
| Inject Autologous REACT | | | | | | | Xⁿ | | |
| CT Scan^{o} | | | | | | | X | | |
| Discharge | | | →→→X^{p} | | | | →→→X^{p} | | |
| Administer KDQOL Survey | X | | | | | | X | | |
| Administer EQ-5D-5L Survey | X | | | | | | X | | |

| | Follow-up First | | | Optional^{b} | | Last REACT Injection | | |
|---|---|---|---|---|---|---|---|---|
| | REACT Injection | | | Month 3 | | | Day 0 | Day 1 |
| | Day 14 (±) | Day 28 (±) | Month 2 (±) | Month 6 (±) | | Day** −14 | REACT | Follow- |
| Clinical Assessment: | 3 days | 3 days | 7 days | 7 days | | to −10 | Injection | up |
| Obtain Informed Consent^{e} | | | | | Interval | | | |
| Verify I/E Criteria | | | | | Between REACT | X | X | |
| Obtain Demographic Data | | | | | Injections = | | | |
| Obtain Medical History | | | | | 3 Months (+) | | | |
| Record ConMeds | X | X | X | X | 12 Weeks** | X | X | X |
| Perform Comprehensive PE^{f} | | | | | | | | |
| Perform Interm PE^{f} | | X | | | | X | | |
| Measure Vital Signs^{g} | X | X | X | X | | X | X^{h} | X |
| Conduct Laboratory Testsⁱ | X | X | X | X | | X | X | X |
| Perform 12-lead ECG | | X | | | | X | | |
| Perform Ultrasound | | | | | | X^{j} | X^{k} | X^{k} |
| Perform MRI Study | | | | | | X | | |
| Perform Renal Scintigraphy | | | | | | X^{m} | | |
| Admit to Hospital | | | | | | | X^{p} | |
| Perform Kidney Biopsy | | | | | | | | |
| Monitor/Record AEs | X | X | X | X | | X | X | X |
| Inject Autologous REACT | | | | | | | Xⁿ | |
| CT Scan^{o} | | | | | | | X | |
| Discharge | | | | | | | →→→X^{p} | |
| Administer KDQOL Survey | | X | X | X | | | X | |
| Administer EQ-5D-5L Survey | | X | X | X | | | X | |

| | Follow-up Last | | | | Follow-up Long-Term | |
|---|---|---|---|---|---|---|
| | REACT Injection | | | | Months 6, | |
| | | | | Month 2 | 9, 12, 15, | |
| | Day 7 (±) | Day 14 (±) | Day 28 (±) | Month 3 (±) | 18, 21 (±) | EOS^{d} |
| Clinical Assessment: | 3 days | 3 days | 3 days | 7 days | 7 days | Month 24 [symbol] |
| Obtain Informed Consent^{e} | | | | | | |
| Verify I/E Criteria | | | | | | |
| Obtain Demographic Data | | | | | | |
| Obtain Medical History | | | | | | |
| Record ConMeds | X | X | X | X | X | X |
| Perform Comprehensive PE^{f} | | | | | | |
| Perform Interm PE^{f} | | | X | | X | X |
| Measure Vital Signs^{g} | X | X | X | X | X | X |
| Conduct Laboratory Testsⁱ | X | X | X | X | X | X |
| Perform 12-lead ECG | | | X | | X | X |
| Perform Ultrasound | | | | | | X^{j} |
| Perform MRI Study | | | | | | X |
| Perform Renal Scintigraphy | | | | | X^{m} | X^{m} |
| Admit to Hospital | | | | | | |
| Perform Kidney Biopsy | | | | | | |
| Monitor/Record AEs | X | X | X | X | X | X |
| Inject Autologous REACT | | | | | | |
| CT Scan^{o} | | | | | | |
| Discharge | | | | | | |
| Administer KDQOL Survey | | | X | X | X | X |
| Administer EQ-5D-5L Survey | | | X | X | X | X |

Abbreviations:
AE (adverse event);
ConMeds (concomitant medications);
DMSB (Data Safety and Monitoring Board);
ECG (electrocardiogram);
EOS (End-of-Study Visit);
I/E (inclusion/exclusion);
KDQOL (Kidney Disease Quality of Life Survey);
MRI (magnetic resonance imaging);
REACT (-Kidney Augment);
PE (physical examination).
Notes:
**Every attempt is made to ensure that the second REACT injection is administered 3 months after the first injection.
a. If the screening assessment falls outside of the 60-day window before renal biopsy, re-screening is performed as described in Screening (Section 6.1).
^{b}Because the second REACT injection occurs 3 months (+12 weeks) after the first injection, the 3-month visit or the 6-month visit may not be scheduled.
c. In the event that a second REACT injection is not administered, the subject undergoes all follow-up assessments after the last REACT injection at the 12-month EOS Visit.
^{d}The EOS Visit takes place 12 months after the last REACT injection, or when the subject is terminated from the study by the Investigator (Section 8.4) or when the subject voluntarily discontinues from the study (Section 5.4).
^{e}The Informed Consent Form must be signed and dated prior to conducting any study-specific procedures, including those at the Screening Visit.
^{f}The PE and interim PE are described in Section 7.2.2).
^{g}Vital signs include heart rate, resting blood pressure, respiration rate, and body temperature. (Section 7.2.1).
^{h}Vital signs are measured throughout the procedure.
ⁱRefer to Table 2 for a schedule of laboratory assessments
^{j}Ultrasound is performed at the Screening Visit to verify subject eligibility, confirm anatomic features of the kidney to be biopsied and injected, and to obtain baseline echogenicity reading. Subsequent Ultrasounds monitor echogenicity.
^{k}Ultrasound is performed following the in-patient renal biopsy on Day 0 and Day 1, and following the in-patient REACT injection(s) on Day 0 and Day 1 with the aim of monitoring possible, subclinical AEs.
^{l}A MRI study without contrast is performed at the Screening Visit through Day −1 before renal biopsy to determine kidney size and volume. This MRI is repeated before the second injection to ensure proper dose calculation. An ultrasound i done to determine kidney size to calculate dose if patient is unable to undergo MRI or if site feels ultrasound is adequate for measurement.
^{m}Renal scintigraphy is performed before the first REACT injection, before the last REACT injection, at the 6-Month Visit after the last REACT injection, and at the EOS Visit.
ⁿThe REACT preparation is handled and injected according to procedures described in the Study Reference Manual.
^{o}CT Scan can be used in conjunction with ultrasound for the REACT injection procedure.
^{p}Subjects who do not experience complications may be discharged the same day consistent with site standard practice. [symbol]
indicates data missing or illegible when filed

TABLE 2
Laboratory Time and Events Table

| | Screening | Renal Biopsy | | | First REACT Injection | | | Follow-up First |
|---|---|---|---|---|---|---|---|---|
| | Visit | | | Day 1 | | | Day 1 | REACT Injection |
| | Day −60 | Day −3 | Day 0 | Follow- | Day −14 | Day 0 | Follow- | Day 7 (±) |
| Laboratory Assessment: | to −3ⁿ | to −1 | Biopsy | up | to −10 | REACT | up | 3 days |

Clinical Chemistry

| | | | | | | | | |
|---|---|---|---|---|---|---|---|---|
| Standard panel | X | X | X | Preparation | X | X | X | X |
| Renal analytes | X | X | X | and Shipment | X | X | X | X |
| Lipid panel | X | | | of REACT | X | | | |
| Pregnancy test^{e} | X | X | | Product* | X | | | |
| FSH test^{f} | X | | | | | | | |

Serology

| | |
|---|---|
| HIV, HBV, HCV | X |

Hematology

| | | | | | | | | |
|---|---|---|---|---|---|---|---|---|
| Standard cell counts/indices | X | X | | X | X | | X | X |
| Hemoglobin, Hematocrit^{g} | | | X^{h} | X^{h} | | X^{h} | X^{h} | |

Coagulation Status

| | | | |
|---|---|---|---|
| Platelet count | X | X | X |
| APTT | X | X | X |
| PT-INR | X | X | X |

Routine Urinalysis

| | | | | | | | |
|---|---|---|---|---|---|---|---|
| Standard panel | X | X | X | X | X | X | X |
| 24-hour | X | | | | | | |
| Test stick | | | X | X | Xⁱ | | |

Additional Tests

| | | | |
|---|---|---|---|
| HbA₁ₑ | X | | |
| Drugs of abuse | X | | X |
| iPTH | X | X | X |
| B2-microglobulin^{j} | X | X | X |
| NGAL | X | X | X |
| Research (reserve) samples^{k} | X | X | X |

| | Follow-up First | | | Optional^{b} | Last REACT Injection | | |
|---|---|---|---|---|---|---|---|
| | REACT Injection | | | Month 3 | | Day 0 | Day 1 |
| | Day 14 (±) | Day 28 (±) | Month 2 (±) | Month 6 (±) | Day** −14 | REACT | Follow- |
| Laboratory Assessment: | 3 days | 3 days | 7 days | 7 days | to −10 | Injection | up |

Clinical Chemistry

| | | | | | | | | |
|---|---|---|---|---|---|---|---|---|
| Standard panel | X | X | X | X | Interval | X | X | X |
| Renal analytes | X | X | X | X | Between REACT | X | X | X |
| Lipid panel | | | | | Injections = | X | | |
| Pregnancy test^{e} | | | | | 3 Months (+) | X | | |
| FSH test^{f} | | | | | 12 Weeks** | | | |

Serology

HIV, HBV, HCV

Hematology

| | | | | | | | |
|---|---|---|---|---|---|---|---|
| Standard cell counts/indices | X | X | X | X | X | | X |
| Hemoglobin, Hematocrit^{g} | | | | | | X^{h} | X^{h} |

Coagulation Status

| | |
|---|---|
| Platelet count | X |
| APTT | X |
| PT-INR | X |

Routine Urinalysis

| | | | | | | | |
|---|---|---|---|---|---|---|---|
| Standard panel | X | X | X | X | X | X | X |
| 24-hour | | | X | X | | | |
| Test stick | | | | X | X | Xⁱ | |

Additional Tests

| | | | | |
|---|---|---|---|---|
| HbA₁ₑ | | | | |
| Drugs of abuse | | | | X |
| iPTH | X | X | X | X |
| B2-microglobulin^{j} | X | | X | X |
| NGAL | X | X | X | X |
| Research (reserve) samples^{k} | X | X | X | X |

| | Follow-up Last | | | | Follow-up Long-Term | |
|---|---|---|---|---|---|---|
| | REACT Injection | | | | Months 6, | |
| | | | | Month 2 | 9, 12, 15, | EOS^{d} |
| | Day 7 (±) | Day 14 (±) | Day 28 (±) | Month 3 (±) | 18, 21 (±) | Month 24 (±) |
| Laboratory Assessment: | 3 days | 3 days | 3 days | 7 days | 7 days | 7 days |
| Clinical Chemistry | | | | | | |
| Standard panel | X | X | X | X | X | X |
| Renal analytes | X | X | X | X | X | X |
| Lipid panel | | | | | | X |
| Pregnancy test^{e} | | | | | | X |
| FSH test^{f} | | | | | | |
| Serology | | | | | | |
| HIV, HBV, HCV | | | | | | |
| Hematology | | | | | | |
| Standard cell counts/indices | X | X | X | X | X | X |
| Hemoglobin, Hematocrit^{g} | | | | | | |
| Coagulation Status | | | | | | |
| Platelet count | | | | | | |
| APTT | | | | | | |
| PT-INR | | | | | | |
| Routine Urinalysis | | | | | | |
| Standard panel | X | X | X | X | X | X |
| 24-hour | | | | X | X | X |
| Test stick | | | | | | |
| Additional Tests | | | | | | |
| HbA₁ₑ | | | | | X | X |
| Drugs of abuse | | | | | | |
| iPTH | | | X | X | X | X |
| B2-microglobulin^{j} | | | X | | X | X |
| NGAL | | | X | | X | X |
| Research (reserve) samples^{k} | | | X | X | X | X |

Abbreviations:
APTT (Activated Partial Thromboplastin Time);
FSH (Follicle Stimulating Hormone;
HbA1c (glycosylated hemoglobin);
HIV (Human Immunodeficiency Virus);
HBV (Hepatitis B Virus);
HCV (Hepatitis C Virus );
NGAL (Neutrophil Gelatinase-Associated Lipocalin);
REACT (Kidney Augment);
i PTH (Parathyroid Hormone, intact);
PT-INR (Prothrombin Time-International Normalized Ratio)
Notes:
*Day 1 Follow up after Biopsy is an optional lab kit to be used only if patient was admitted and kept overnight in hospital per site standard practice
**Every attempt is made to ensure that the second REACT injection is administered 3 months after the first injection.
a. If the screening assessment falls outside of the 60-day window before renal biopsy, re-screening is performed as described in Screening (Section 6.1).
^{b}Because the second REACT injection occurs 3 months (+12 weeks) after the first injection, the 3-month visit or the 6-month visit may not be scheduled.
c. In the event that a second REACT injection is not administered, the subject undergoes all follow-up assessments after the last REACT injection at the 12-month EOS Visit.
^{d}The EOS Visit takes place 12 months after the last REACT injection, or when the subject is terminated from the study by the Investigator (Section 8.4 ), or when the subject voluntarily discontinues from the study (Section 5.4 ).
^{e}The clinic performs a urine dip-strip pregnancy test. If positive, then a confirmatory test is performed by the central laboratory.
^{f}Post-menopausal women with a confirmatory FSH test do not have to undergo pregnancy testing throughout the study.
^{g}Within 24 hours before Days 0 for renal biopsy and REACT injection(s), hemoglobin levels are verified as >9 g/dL per site standard practices.
^{h}On Days 0 for renal biopsy and REACT treatment (s), hemoglobin and hematocrit are measured before and after procedure per site standard practice at the local. These samples are processed by the site's local laboratory to accelerate notification of results and subsequent decisions affecting clinical care. Additionally, blood samples for hemoglobin and hematocrit after procedure are sent to the central laboratory where results can be entered into the study database.
ⁱPrior to REACT injection, microscopic urinalysis are performed to confirm the absence of infection using a dip stick
^{j}β2-microglobulin is assessed in both serum and urine samples.
^{k}Research samples (serum/plasma and urine) are collected, frozen, and stored for the evaluation of novel biomarkers.

TABLE 3
Abbreviations and Specialist Terms

| Abbreviation or | |
|---|---|
| Specialist Term | Explanation |
| ACEI | Angiotensin-Converting-Enzyme Inhibitor |
| AE | Adverse Event |
| ALT | Alanine Transaminase |
| APTT | Activated Partial Thromboplastin Time |
| ARB | Angiotensin Receptor Blocker |
| AST | Aspartate Transaminase |
| BMI | Body Mass Index |
| BP | Blood Pressure |
| BUN | Blood Urea Nitrogen |
| CAKUT | Congenital Anomalies of the Kidney and Urinary |
| | Tract |
| CKD | Chronic Kidney Disease |
| ConMed(s) | Concomitant Medication(s) |
| CRF | Case Report Form |
| CRP | C-Reactive Protein |
| CTCAE | Common Terminology Criteria for Adverse Events |
| DMSA | Dimercaptosuccinic acid |
| ECG | Electrocardiogram |
| EC | Ethics Committee |
| eGFR | Estimated Glomerular Filtration Rate |
| EOS | End-of-Study |
| ESRD | End Stage Renal Disease |
| FDA | Food And Drug Administration |
| G | Gram(s) |
| GCP | Good Clinical Practice |
| GFR | Glomerular Filtration Rate |
| gKW^{est} | Gram(s) of Estimated Kidney Weight |
| GLP | Good Laboratory Practice |
| GMP | Good Manufacturing Practice |
| Hb | Hemoglobin |
| HbA_{1c} | Glycosylated Hemoglobin |
| HBV | Hepatitus B Virus |
| HCV | Hepatitus C Virus |
| HIV | Human Immunodeficiancy Virus |
| HR-QoL | Health-Related Quality of Life |
| IB | Investigator's Brochure |
| ICH | International Conference On Harmonization |
| I/E | Inclusion/Exclusion |
| IND | Investigational New Drug |
| INR | International Normalization Ratio |
| iPTH | Intact Parathyroid Hormone |
| KDQOL | Kidney Disease Quality-of-Life Survey |
| MedDRA | Medical Dictionary for Regulatory Activities |
| Mo | Month |
| MRI | Magnetic Resonance Imaging |
| NCI | National Cancer Institute |
| NKA | Neo Kidney Augment |
| NOAEL | No-Observed-Adverse-Effect-Level |
| NSAIDs | Non-Steroidal Anti-Inflammatory Drugs |
| Nx | Nephrectomy |
| PE | Physical Examination |
| PI | Principal Investigator |
| PT | Prothrombin Time |
| PT-INR | Prothrombin Time-International Normalization Ratio |
| QA | Quality Assurance |
| REACT | Renal Autologous Cell Therapy |
| RBC | Red Blood Cell |
| SAE/SAR | Serious Adverse Event/Serious Adverse Reaction |
| sCr | Serum Creatinine |
| SPIO | Superparamagnetic Iron Oxide |
| SRC | Selected Renal Cells |
| SUSAR | Suspect, Unexpected Serious Adverse Reaction |
| T2DM | Type 2 Diabetes Mellitus |
| TB | Tuberculosis |
| UACR | Urinary Albumin/Creatinine Ratio |
| WBC | White Blood Cell |

### 1. CAKUT and Chronic Kidney Disease

A common component of CAKUT is vesicoureteric reflux is defined as the back flow of urine from the urinary bladder into one or both ureters, the renal pelvises, or both. Primary vesicoureteral reflux (VUR) is the commonest congenital urinary tract abnormality in childhood, which is diagnosed mostly after an episode of urinary tract infection (UTI). VUR is believed to predispose to urinary tract infection (UTI) and renal scarring. Renal scarring associated with VUR is also known as reflux nephropathy (RN). Long-term potential complications of RN include hypertension, proteinuria, and progression to end-stage renal disease (ESRD) [5]. Patients with abnormally developed kidneys are most vulnerable to development of ESRD as kidneys continue to worsen even after VUR has been corrected (Brakeman). Ardissino et al. found that nearly 26% of end stage renal disease in patients with hypodysplasia was associated with vesicoureteral reflux. The exact incidence of RN in children or adults is not known. RN is responsible for 12% to 21% of all children with chronic renal failure [1, 2]. According to the 2008 North American Pediatric Renal Trials and Collaborative Studies report, RN is the fourth commonest cause for chronic kidney disease in 8.4% of the children and is the primary pathology in 5.2% of transplanted patients and 3.5% of dialysis patients [3]. In the CKID study that involved a cohort of 586 children aged 1 to 16 years with an estimated GFR of 30 to 90 mL/min/1.73 m². RN was the underlying cause for CKD in 87 (14.8%) patients. In adults, obstructive uropathy accounted for 0.3% of the point prevalent cases of ESRD for 2005, a fraction of which may be due to RN. Few cohort studies have performed long-term follow-up of patients post-antireflux surgery. In one cohort from Israel, only 1 of 100 patients developed CKD after 20 years. In another cohort of patients identified as having renal scarring by IVP, 18% developed CKD after 20 years. Regardless, the incidence of ESRD in adults due to RN is low.

Chronic Kidney Disease (CKD) is characterized by progressive nephropathy that without therapeutic intervention will worsen until the patient reaches ESRD. CKD is defined as reduced kidney function, demonstrated by a decreased glomerular filtration rate (GFR) or evidence of kidney damage, such as increased excretion of urinary albumin. Global prevalence of CKD is estimated at 8-16%. To survive, ESRD patients require renal replacement therapy (dialysis or kidney transplantation). Preventing or delaying adverse outcomes of CKD via early intervention is the primary strategy in CKD management. Nevertheless, early treatments have been less than optimal, resulting in a significant unmet medical need for improved interventional strategies to manage CKD and delay progression to ESRD.

Treatment of patients with CKD is focused on slowing progression and preparing for kidney failure/replacement. For many patients, CKD occurs as part of a complex comorbidity cluster. When a patient reaches ESRD, renal replacement therapy (i.e., dialysis or transplantation) is indicated. The vast majority of Stage 5 patients receive hemodialysis.[4] Dialysis replaces about 5-15% of kidney function, depending on the intensity and frequency of use; dialysis also helps to restore fluid and electrolyte balance when kidneys fail. However, the life expectancy of an ESRD patient initiating hemodialysis is only 4-5 years.[5] Additionally, hemodialysis has been associated with multiple, serious complications as well as interference with quality of life, such as the need to undergo dialysis up to three times per week. Although kidney transplantation remains the most effective form of therapy at this time; there is a chronic shortage of organs. If a patient is able to secure a kidney for transplantation, long-term immunosuppressive therapy is required to prevent rejection. Use of these regimens results in a higher incidence of infection and, over the long term, some types of cancer.[6] Taken together, there is a critical medical need for improved therapies for CKD that could dramatically slow the progression of disease and significantly delay, or reduce the need for renal transplantation. Table 4 defines CKD stages according to GFR measurements. The initial stage of nephropathy (Stage 1) occurs over a period of several years and is characterized by microalbuminuria (30-300 mg/24 hr) followed by macroalbuminuria (>300 mg/24 hr). As the ability of the kidney to filter blood waste products declines, serum creatinine rises. With increasing kidney damage (Stages 2-4), rising blood pressure further exacerbates kidney disease. When the kidneys cease to function entirely (Stage 5 [ESRD]), renal replacement therapy (dialysis or transplantation) is required.

TABLE 4
Summary of Classification and Prevalence Estimates for CKD

| Stage* | Description | GFR (mL/min/1.73 m²) |
|---|---|---|
| 1 | Kidney damage with normal or | ≥90 |
| | increased GFR | |
| 2 | Kidney damage with mild | 60-89 |
| | decrease in GFR | |
| 3 | Moderate decrease in GFR | 30-59 |
| 4 | Severe decrease in GFR | 15-29 |
| 5 | Kidney failure | <15 (or dialysis) |

*Source: National Kidney Foundation, 2002. National Kidney Foundation. 2002. KDOQI clinical practice guidelines for chronic kidney disease: evaluation, classification, and stratification. Am J Kidney Dis. 39:S1-266.

1.1 Non-Clinical Pharmacology Studies

In a series of pre-clinical studies, Tengion (a former regenerative medicine company) defined the pharmacological characteristics of SRC and delayed the progression of experimental models of CKD by augmenting renal structure and function. [7-12] Tengion subsequently conducted safety pharmacology and GLP toxicology studies. An overview of these non-clinical studies is presented in Table 5.

TABLE 5
Summary of Non-clinical Pharmacology and Toxicology Studies

| | | | Dose per | | Total | | | | |
|---|---|---|---|---|---|---|---|---|---|
| | | | Kidney | Number | Kidney | Total^{b} | Total | SRC | |
| | Number | Study | Weight^{a} | Kidneys | Weight (g) | Dose | SRC × | Conc. | |
| Model | Animals | Length | 10⁶Cell/g | Injected | Injected^{a} | (mL) | 10⁶ | 10⁶/mL | Study |

Pharmacology (efficacy, kinetics, migration, and persistence)

| | | | | | | | | | | |
|---|---|---|---|---|---|---|---|---|---|---|
| 5/6 Nephrectomy | 3 | 6 | mo | 5-10 | 1 (1) | 1 | 0.1 | 5-10 | 50-100 | 1 |
| Lewis Rat^{d} | | | | | | | | | | |
| 70% Nephrectomy | 4 | 10 | mo | 6 | 1 (2) | 57.7 | 5 | 334 | 66.8 | 2 |
| Canine^{d} | | | | | | | | | | |
| ZSF-1^{e} | 7 | 12 | mo | 3 | 2 (2) | 3.2 | 0.4 | 10 | 25 | 3 |
| 5/6 Nephrectomy | 77 | 4 | days | 5-15 | 1 (1) | 1 | 0.1 | 5-15 | 50-150 | 4 |
| Lewis Rat^{e} | | | | | | | | | | |
| Canine^{e} | 1 | 30 | min | 12.5 | 2 (2) | 120 | 10 | 1500 | 150 | 5 |
| Canine^{e} | 4 | 30 | min | 1.5-9.2 | 2 (1-2) | 120 | 2.5-10 | 92.77-553.5 | 37-55 | 6 |

GLP Toxicology

| ZSF-1 rat^{d} | | 5M/5F | 3 | mo | 3.13 | 2 (2) | 2 | .25 | 6.25 | 25 | 7 |
|---|---|---|---|---|---|---|---|---|---|---|---|
| | | 5M/5F | 6 | mo | 3.13 | 2 (2) | 2 | .25 | 6.25 | 25 | |
| | | 5M/5F | 3 | mo | 6.25 | 2 (2) | 2 | .25 | 12.5 | 50 | |
| | | 5M/5F | 6 | mo | 6.25 | 2 (2) | 2 | .25 | 12.5 | 50 | |
| Canine^{e} | | 2M/2F | 1 | mo | 2.75 | 1 (1) | 60 | 3 | 330 | 110 | 8 |
| | | 2M/2F | 3 | mo | 2.75 | 1 (1) | 60 | 3 | 330 | 110 | |
| | | 2M/2F | 1 | mo | 11.0 | 2 (1) | 120 | 12 | 1320 | 110 | |
| | | 2M/2F | 3 | mo | 11.0 | 2 (1) | 120 | 12 | 1320 | 110 | |
| Canine^{c,e} | 0 months | 2M/2F | n/a | | 5.5 | 2 (2) | 120 | 6 | 660 | 110 | 9 |
| | 3 months | | 6 | mo | 5.5 | 2 (2) | 120 | 6 | 660 | 110 | 10 |

Notes:
^{a}Estimated kidney weight based on animal model. Actual weights are listed in the study reports where applicable.
^{b}Dose refers to SRC or REACT.
^{c}Two doses of REACT were administered, one at 0 months and the second at 3 months.
^{d}Delivered REACT to rodents using syringe affixed to sharp needle that pierced the capsule and deposited REACT.
^{e}Delivered REACT to dogs using cannula to pierce the capsule plus blunt-end delivery cannula to deposit REACT.

1.1.1. Pharmacodynamics

Proof of principle for SRC as the biologically active component of REACT was established in multiple animal models of CKD. For example, the ⅚th nephrectomy (Nx) rodent mass reduction model of CKD allowed for an optimized selection of a therapeutically relevant SRC cell population. A 70% Nx canine model of CKD confirmed SRC activity in a large mammal, while the ZSF-1 rat served as proof-of-principle for demonstrating the effects of SRC in a model relevant to T2DM.[13] SRC delivered directly into the kidney cortex in multiple experimental models of CKD induced a regenerative response through direct engraftment or tissue replacement, and also induced secretory factors via a putative paracrine mechanism.[9, 14-17]

This intervention strategy significantly improved survival, stabilized disease progression, and extended the longevity in both the ⅚th Nx model and the ZSF-1 rodent models of CKD. Morphological normalization of multiple nephron structures was accompanied by functional improvements, including glomerular filtration, tubular protein handling, electrolyte balance, and the ability to concentrate urine. Lowered blood pressure and reduced levels of circulating renin were also observed in the ZSF-1 rat model. The observed functional improvements following SRC treatment were accompanied by significant reductions in glomerular sclerosis, tubular degeneration and interstitial inflammation and fibrosis. No toxicologically significant in-life, clinical pathology, or histological changes were noted in the target organ or other tissues. Based on results from multiple pre-clinical studies conducted in different CKD animal models, SRC (ie, active component of REACT) were effective in significantly delaying progression of CKD when injected in the diseased organ prior to irreversible nephropathy. These results provide a rationale for investigating the effects of this cell-based intervention in patients prior to ESRD.

1.1.2. Safety Pharmacology

1.1.2.1. Extra-Renal Activity

REACT (i.e., SRC formulated in a gelatin-based hydrogel) was administered in various rat and canine models to assess immediate cardiovascular and respiratory pharmacologic effects. The acute effects of lower and higher SRC concentrations formulated in varying percentages of gelatin (0.75-1.0%) were evaluated in the rodent ⅚th Nx model. Potential changes in blood pressure were assessed immediately before, during, and shortly after REACT delivery in the normal canine model. No studies on the effects of REACT on the central nervous system were performed since: 1) animals exhibited normal behavior before, during, and after REACT injection; 2) no effects on the central nervous system were expected from an investigational product containing intact renal cells; and, 3) REACT was delivered into the kidney.

1.1.2.2. Hemodynamic Effects

Rats in the ⅚th Nx study (Study #4) received REACT or vehicle control, and potential hemodynamic effects were monitored over 4 days. Among the 77 animals treated in this REACT formulation study, 16 animals experienced apnea during or immediately after REACT delivery. A total of 9 animals died; the causes of death were classified as apnea (n=3), renal hemorrhage (n=2), and deaths associated with CKD (n=4). Six of the 16 animals that experienced apnea were not pre-treated with atropine; of these animals that experienced apnea, two died under the influence of anesthesia prior to the use of atropine. Ten of the 16 animals that experienced apnea were treated with atropine, and all recovered from the surgical procedure and REACT injection.

On the other hand, apnea, renal hemorrhage, and deaths that occurred in the ⅚th Nx rat study were not observed in the ZSF-1 rat study, or in the canine pharmacology study, or in two (intact) canine pilot studies that assessed the short-term effects of volume administration on blood pressure. Without being bound by any theory, taken together, this model-specific hemodynamic response can be potentially attributed to: 1) altered hemodynamics of the severely mass-reduced rodent remnant kidney;[18] 2) transient changes in kidney interstitial pressure administration triggering a central autonomic response;[19, 20] and, 3) under perfusion of tissue or acute hypoxia from bleeding following delivery into the kidney. Pre-treatment with atropine, a competitive antagonist of the parasympathetic nervous system, helped mitigate the model-specific hemodynamic changes. The effect of atropine suggested a possible autonomic response to REACT delivery that was specific to the severely mass-reduced, ⅚th Nx rodent model of CKD.

1.1.2.3. Dose Volume

Using a range of REACT doses, volumes, and concentrations, (Study #5), the normal canine was selected to evaluate blood pressure immediately before, during, and after REACT delivery into the kidney. In this study, each pole of each kidney received 2.5 mL of REACT; therefore, a total of 10 mL/120 g, or 0.083 mL/g, was delivered at a dosage of 12.5×106 cells/gram of kidney mass. REACT treatment was well tolerated; there were no adverse systemic effects (physical or serological), nor were there any significant toxicological or histomorphological changes indicative of kidney injury or other tissue injury as a result of REACT delivery. In contrast to the severe mass reduction rodent model of CKD, no apnea, renal hemorrhage, or deaths were noted.

1.1.3. Kinetics, Migration, and Persistence

As with other cell-based therapies targeting soft organs, data on biodistribution of the investigational product has been limited. To that end, three additional studies provide evidence concerning the potential migration and persistence of REACT within the kidney at selected sampling times post-delivery.[16, 17, 21] The results of these studies are summarized in this section; detailed information is provided in the Investigator's Brochure.

1.1.3.1. ZSF-1 Rat

SRCs were labeled with the Rhodamine-B superparamagnetic iron oxide (SPIO) particle. This contrast agent is specifically formulated for cell labeling and is readily internalized by non-phagocytic cells. SPIO-labeled cells were administered to the ZSF-1 rat kidney. Twenty-four hours after delivery, SPIO-labeled cells were detected by MRI and whole organ optical imaging. In addition, ZSF-1 rats received SRCs labeled with CelSense-19F, which were quantified by Nuclear Magnetic Resonance at 3 hr, 24 hr, and 7 days after injection.[16, 22]

Both acute ZSF-1 detection and long-term donor cell detection using the ⅚th Nx model of CKD showed significant retention of SPIO-labeled cells. Clinically relevant MRI detection at 24 hr following cell delivery revealed a region at the anterior pole of the kidney where SPIO-labeled cells had been injected. Sectioning of the whole kidney and staining with Prussian blue demonstrated a bolus of iron-labeled SPIO cells migrating and distributing from the cortical injection site, which confirmed their presence in tubular and peritubular spaces of the renal cortex and medulla.

Likewise, whole organ fluorescent imaging highlighted cell detection at and around the site of injection located at the upper cortex of the anterior pole of the ZSF-1 rat kidney. Detection of 19F-labeled SRC at 3 and 24 hr after delivery confirmed nearly 100% retention in the kidney. After 7 days, detection of 19F-labeled SRC was diminished by an order of magnitude, consistent with continuous urinary excretion.

1.1.3.2. Porcine Non-GLP Analysis of Non-Renal Tissue

In a pre-clinical, non-GLP study, SPIO-labeled SRC were delivered to the kidneys of living swine (n=11). Cellular distribution was monitored over the course of the 30-day study period using MRI. Labelled SRC were distributed in two major compartments: urinary bladder and renal parenchyma. The major route of excretion was urine. Notably, there was no evidence of ectopic SRC migration or site-specific engraftment at non-target organ sites.[23]

1.1.3.3. Canine Non-GLP Analysis of Non-Renal Tissue

In a pre-clinical, non-GLP study, SPIO-labeled SRC were delivered to the kidneys of living canine hosts. Cellular distribution was monitored at 30 minutes post-injection via MRI.

Consistent with observations from the living porcine model demonstrating that injected SPIO-labeled SRC was retained in renal parenchyma or excreted in urine, SPIO-labeled SRC likewise were retained within the renal parenchyma at the injection site after 30 minutes.

1.1.3.4. Conclusions
SRC were distributed at the site of injection (renal parenchyma) and excreted via the urine, based on SRC labeling studies with SPIO and CelSense-19F.
SRC delivered into rat, swine, and canine kidneys were not detected in non-target organs (other than urinary tract during excretion), based on extensive histological evaluation.
Based on reports concerning allogenic mesenchymal stem cells as well as published data concerning the safety of autologous mesenchymal stem cells in clinical trials, autologous REACT-related materials should also not give rise to ectopic tissue growth, organ dysfunction, or tumor development.[24-28]

1.2. Toxicology Studies

To assess the safety of REACT, three GLP safety studies were conducted, ie, one study was conducted in the rat ZSF-1 disease model of CKD and the other two studies were conducted in normal canines.

1.2.1. ZSF-1 Rat Single Dose Study

The purpose of this study was to assess the safety of a single administration of REACT in ZSF-1 rats, a model of uncontrolled metabolic syndrome including T2DM, hypertension, and severe obesity. The rats received: 1) high dose REACT; 2) low dose REACT; 3) sham; or, 4) biomaterial only. Each animal received 4 injections of test article, one into each pole of each kidney. The results were assessed at 3 and 6 months post-treatment.

1.2.1.1. Renal-Related Findings

No treatment-related kidney findings were noted following evaluation of 8 areas of each kidney (3 stains per area), including assessment and scoring of 150 glomeruli per kidney. Apart from changes related to injection and/or injection site linear scars, all kidneys were considered normal within the context of the disease model. No test article-related kidney finding was observed at 3 or 6 month post-treatment. All macroscopic and microscopic kidney changes were considered related to the natural progression of renal disease in the ZSF-1 obese rat, or to the injection procedure.

Kidney changes in all groups were more severe in males, and consistent with differences in the disease stage between genders. Overall, there was an apparent trend of lower renal histological severity scores (ie, lower glomerular injury score, tubule-interstitial injury score, and global nephron score) that was consistently noted in the low-concentration REACT treatment group when compared to the Sham control group 6 months post-procedure.

Based on the absence of differences across study groups, the No-Observed-Adverse-Effect-Level (NOAEL) was the high dose, 6.25×106 cells/g KWest.

1.2.1.2. Non-Renal Findings

No REACT safety-related findings were observed in non-target tissues. No ureteral or bladder (primary routes of REACT excretion) REACT-related changes were observed. There were no REACT-related effects, and no observable REACT cellular materials in any of the draining (lymph nodes) or filtering (liver, lung, spleen) tissues examined.

1.2.1.3. Clinical Pathology

The results of clinical laboratory tests (including hematology, clinical chemistry, and special urinalysis panels) were evaluated for differences between baseline and end of study (3 or 6 months post-treatment), and between treated and control groups. No REACT-related clinically significant laboratory abnormalities were identified.

1.2.1.4. Conclusions
All animals survived to the end of study (3 or 6 months post-treatment).
There were no significant safety-related clinical pathology findings or safety-related findings of toxicological significance attributable to treatment.
No REACT-related clinically significant laboratory abnormalities were identified.
The observed NOAEL was 6.25×106 cells/g KWest.

1.2.2. Initial Single Dose Canine Study

The initial canine toxicology study assessed the safety of a single administration of two different doses of REACT compared to sham treatment or treatment with the biomaterial. The test article was delivered into one pole of each kidney. A total of 32 mongrel dogs were entered into the study; 16 were assessed at one month and 16 were assessed at 3 months.

1.2.2.1. General Results

All 32 animals survived to their designated termination time point at one or 3 months after treatment. The animals appeared to be in good health throughout the study. There were no significant clinical pathology findings. There were no signs of renal insufficiency (azotemia), and there were no indications of decreased GFR.

1.2.2.2. Renal-Related Results

No REACT delivery-related macroscopic or microscopic findings were observed at the one or 3 month endpoint. No treatment-related kidney findings were noted following enhanced evaluation of 8 areas of each kidney (3 stains per area), including evaluation and scoring of 150 glomeruli per kidney. Apart from changes related to injection site scars all kidneys were normal. All macroscopic and microscopic kidney changes were considered background findings or related to the injection procedures.

1.2.2.3. Non-Renal Findings

No test article-related findings were identified in other (non-kidney) tissues. All macroscopic and microscopic changes were considered background changes and within normal limits.

1.2.2.4. Procedure-Related Findings

The most common abnormalities included swelling at the incision sites (seroma formation) and weight loss at study termination. With regard to incision site swelling, ten of sixteen (10/16) animals had sterile seroma formation post-injection, and nine of sixteen (9/16) post treatment.

The animals had varying degrees of swelling at their retroperitoneal incisions, and were treated as deemed necessary by a veterinarian.

Numerous animals had mild in appetence following REACT administration (29 of 32) and treatment procedures (18 of 32). Most animals (28 of 32) experienced weight loss from baseline (prior to injection) to termination. Of note, a greater amount of weight loss occurred between baseline (2 weeks prior to treatment) and treatment (Day 0; renal injections) than between treatment and termination. Weight loss also occurred across all treatment groups and was judged to be related to the stressful nature of the study.

1.2.2.5. Conclusions
All animals survived to their designated termination time point and were in good general health throughout the study based on clinical pathology, urinalysis, and veterinary assessment.
Neither the low or high dose of REACT produced macroscopic or microscopic adverse effects at one or 3 months post-treatment, similar to observations following sham treatment or treatment with the biomaterial.
Pathological evaluation showed no REACT safety-related (macroscopic or microscopic) findings in the target (kidney) or non-target organs examined.
Based on anatomic pathology, the observed NOAEL was the higher concentration tested, ie, 11.7×106 cells/g KWest.

1.2.3. Repeat Dose Canine Study

The second canine toxicology study assessed the safety of administering two repeat doses of REACT. Each dose was delivered into both kidneys at baseline (time zero) and 3 months. All animals were subjected to two renal biopsies per kidney 4 to 6 weeks prior to the baseline injection procedure. Control animals were injected with PBS. Animals were monitored for 6 months following the baseline injection.

1.2.3.1. Study Results

All 8 animals were in good clinical health throughout the study and survived to their designated termination time point at 6 months. There was mild or insignificant weight loss in 5 of 8 animals, with 2 animals losing >3% body weight for the duration of the study. Greater weight loss occurred between the renal injection and initial treatment than between the initial treatment and termination. The clinical pathology and urinalysis data revealed no abnormal trends. There were no signs of renal insufficiency, and no indications of decreased GFR.

1.2.3.2. Kidney-Related Findings

No REACT injection safety-related macroscopic or microscopic findings were observed at the 6-month time point. No treatment-related kidney findings were noted following enhanced evaluation of 8 areas of each kidney (3 stains per area), including evaluation and scoring of 150 glomeruli per kidney. All kidneys appeared normal, apart from changes related to injection site scars (fibrosis/chronic inflammation in the capsule; linear fibrosis/chronic inflammation and inflammatory cells in the cortex/medulla).

1.2.3.3. Non-Kidney Related Findings

No test article safety-related findings were identified in non-target tissues. All macroscopic and microscopic changes were considered background changes and thus, considered within normal limits.

1.2.3.4. Conclusions
All animals survived to their designated termination time point, and appeared in good health based on clinical pathology, urinalysis, and veterinary assessment data.
Pathological assessment showed no REACT safety-related (macroscopic or microscopic) findings in either the target organ (kidney) or non-target organs examined.
At the 6-month time point, no detrimental effects of two repeat doses of REACT were observed in comparison to the control animals injected with PBS.

1.3. Non-clinical Conclusions

Evidence from multiple animal studies over a wide range of doses (3 to 15 million SRC/g of kidney tissue injected), and extended periods of time post-REACT treatment (up to one year), including three GLP studies, indicated that the potential risk of complications from REACT delivery into the kidney was similar to the potential risk of complications associated with standard renal biopsy practice.[1-3]

Apart from changes related to injection procedures and cardiovascular findings specific to ⅚th nephrectomy rodent mass reduction model of CKD, no unanticipated in-life, hematological, urological, serological, or histological changes were found in the target organ or non-target tissues following delivery of REACT.

1.4. Phase 1 Clinical Trial: Interim Results

1.4.1.

In April 2013, a first-in-human clinical trial was initiated at the Karolinska University Hospital Huddinge in Stockholm, Sweden: A Phase 1, Open-Label Safety and Delivery Optimization Study of an Autologous-Kidney Augment (REACT) in Patients with Chronic Kidney Disease (RMTX-CL001). This is a Phase 1, open-label, safety and delivery optimization study of REACT injected into subjects with CKD. REACT is manufactured from SRC obtained from a subject's renal biopsy, formulated with gelatin biomaterial, and injected back into the subject's left kidney. The primary objective is to assess the safety and optimal delivery of REACT injected at one site in a recipient kidney as measured by procedure- and/or product-related adverse events (AEs) through 12 months post-treatment. The secondary objective is to assess renal function by comparing the results of laboratory tests from baseline through 12 months following REACT injection, followed by an additional observational period of 18 months. Each subject's baseline rate of CKD disease progression serves as his/her own “control” to monitor for changes in renal insufficiency over time. Six subjects, recruited from the Karolinska University Hospital, were enrolled into the study. In addition, one subject was enrolled in the study at the University of North Carolina.

1.4.2. Adverse Events

Among a cohort of 7 male subjects, 53 to 70 years of age, with pre-dialysis diabetic nephropathy (Stage 3b/4 of CKD), all subjects recovered from the laparoscopic REACT delivery procedure without immediate perioperative complications. Notably, no subject experienced hematuria, which was prospectively considered to be the most likely untoward event. One subject developed an intestinal volvulus on Day 2 after REACT injection, and required a partial colonic resection that was complicated by anastomotic hemorrhage. In the judgment of the Investigator, this event was not related to the investigational product or the procedure. One subject experienced a skin infection that was associated with the laparoscopic injection procedure. Another subject recovered from the surgical procedure with inflammation of the respiratory tract. All serious adverse events (SAEs) associated with the clinical trial are presented in Table 6.

Eight of the nine SAEs were considered possibly related to the injection surgery. There were no AEs or SAEs considered related to the biopsy procedure. To date, no delayed or late-onset adverse reactions related to REACT or other study procedures have been identified (e.g., negative immune mediated reactions). Based on the current data, the highest risk associated with REACT treatment appears to be from the injection surgery. Consequently, steps are being taken to decrease the duration of the surgical procedure and improve surgical outcomes.

TABLE 6
Serious Adverse Events Reported in Study RMTX-CL001

| Subject | MedDRA | | Time | | Relationship |
|---|---|---|---|---|---|
| Number | Preferred Term | Intensity | to Onset | Outcome of SAE | to REACT |
| 001-002 | Fatigue | Mild | 4 days | Recovered/Resolved | Not Related |
| 001-001 | Fatigue | Mild | 5 days | Recovered/Resolved | Not Related |
| 001-001 | Postoperative | Mild | 20 days | Recovered/Resolved | Possibly Related |
| | wound | | | | |
| | infection | | | | |
| 001-002 | Pneumonia | Moderate | 41 days | Recovered/Resolved | Possibly Related |
| 001-003 | Urinary tract | Mild | 7 days | Recovered/Resolved | Not Related |
| | infection | | | | |
| 001-004 | Fatigue | Mild | 4 days | Recovered/Resolved | Not Related |
| 001-005 | Volvulus | Moderate | 2 days | Recovered/Resolved | Not Related |
| 001-003 | Fluid retention | Moderate | 61 days | Recovered/Resolved | Not Related |
| 001-005 | Anastomotic | Moderate | 40 days | Recovered/Resolved | Not Related |
| | hemorrhage | | | | |

1.4.3. Estimated Glomerular Filtration Rate (eGFR)

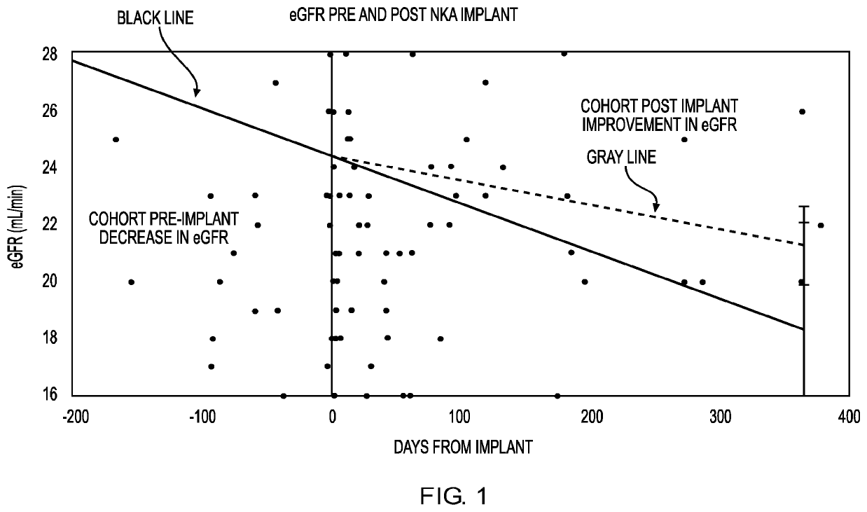

Seven male patients with T2DM and Stage 3b/4 of CKD were injected with REACT in the left kidney. Pre-injection information from these subjects indicated that their average decline in eGFRC was 6.1 ml/min/year. Following REACT treatment, eGFR decline for the combined group (all subjects) was −3.1 mi/min/year (gray line in FIG. 1).

After monitoring the potential impact of REACT treatment on CKD progression in this cohort for approximately one year, the expected decline in renal function appears to have been modified by a single injection of REACT into a single kidney. In FIG. 1, a comparison of eGFR following REACT treatment (gray line) versus eGFR before REACT treatment (black-line) showed that 6 of 7 subjects had a reduction in the rate of eGFR decline post-treatment. The annual rate of change for eGFR, before and after REACT treatment, is presented for each subject in Table 7.

TABLE 7
Estimated Glomerular Filtration Rate by Subject (RMTX-CL001)

| | Change in eGFR (mL/min/year) | |
|---|---|---|
| Subject Number | Pre-REACT | Post-REACT |
| 001-001 | −14.8 | 1.5 |
| 001-002 | −0.2 | −1.3 |
| 001-003 | −6.7 | −5.9 |
| 001-004 | −16.3 | −7.5 |
| 001-005 | −3.9 | −2.6 |
| 001-006 | −11.4 | −5.9 |
| 001-007 | −7.7 | 1.4 |

1.4.4. Serum Creatinine

Pre-treatment levels of serum creatinine (sCR) were generally elevated in this cohort, which would be expected from subjects with T2DM and moderate to severe renal insufficiency (Stage 3b/4 of CKD). The annual rate of change for sCR, before and after REACT treatment, is presented for each subject in Table 8. All subjects demonstrated a reduction in their individual rate of increase for sCr following REACT treatment compared to the rate of sCr increase that had been observed before REACT treatment.

TABLE 8
Serum Creatinine by Subject (RMTX-CL001)

| Subject | Change in Serum Creatinine (μmole/L/year) | |
|---|---|---|
| Number | Pre-REACT | Post-REACT |
| 001-001 | 153 | −41 |
| 001-002 | 17 | −21 |
| 001-003 | 214 | 200 |
| 001-004 | 16 | −39 |
| 001-005 | 69 | 23 |
| 001-006 | 216 | 95 |
| 001-007 | 48 | −40 |

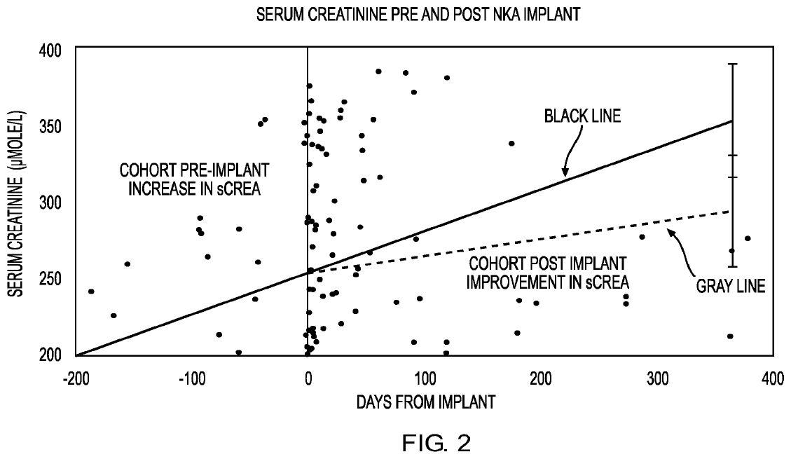

The collective pre-treatment level of sCR for this cohort was >100 μmole/L/yr. Following REACT treatment, sCR decreased to <50 μmole/L/yr. As shown in FIG. 2, a comparison of sCR after REACT treatment (gray line) versus sCr before REACT treatment (black-line) showed that the cohort experienced a reduction in the rate of increase for sCr post-REACT treatment. This change was consistent for each subject.

1.4.5. Kidney Cortical Thickness

Patients suffering from chronic kidney disease undergo a thinning of the functional portion of the kidney, i.e., the cortex. Renal cortical thickness is reduced in CKD as a result of fibrosis and scarring as the disease progresses. An increase in cortical thickness was associated with kidney regeneration in pre-clinical studies of REACT and was confirmed histologically in all 4 animal species studied. In the clinical trials TNG-CL010 and TNG-CL011, cortical thickness was evaluated using imaging technologies; no biopsies were taken to confirm the basis for the increased thickness. Cortical thickness was measured in both the right and left kidney to determine if the injected left kidney exhibited any change in cortical thickness that could be attributed to REACT injection. The right kidney served as a non-injected control.

On average, cortical thickness increased in the left kidney from 14 mm at baseline to approximately 16 mm after one year of REACT treatment. This change in cortical thickness was not sufficient to cause an increase in the total volume of the left kidney (data not presented). No change in cortical thickness was observed in the right kidney cortex.

1.4.6. Hemoglobin

CKD can be associated with anemia due to an alteration in renal erythropoietin production as well as metabolic abnormalities resulting from chronic uremia.[29] In the clinical trial RMTX-CL001, 3 of 7 subjects exhibited improvement in hemoglobin levels after REACT treatment, while the remaining 4 subjects maintained normal levels during the study.

1.4.7. Blood Pressure

Blood pressure was monitored during the course of clinical trials TNG-CL010 and TNG-CL011. Subjects received medication to control their blood pressure. Notably, intake of antihypertensive medication was reduced in 3 of 6 subjects during the first six months following REACT treatment.

1.5. Potential Risks

In general, potential risks associated with the clinical use of REACT can be broadly divided into 3 categories: kidney biopsy, REACT product, and delivery into the recipient kidney. An assessment of potential risks associated with each of these steps is presented in this Section.

At this time, there are no specific warnings or precautions associated with the use of REACT. However, warnings and precautions for a renal biopsy and the percutaneous injection procedure must be considered with use of this product. The risks of renal biopsy have been well characterized in the 100 years that this procedure has been used and developed. Percutaneous needle instrumentation of the kidney has a shorter history.

The risks of renal biopsy and percutaneous needle kidney injection include:
1. Pain in flank/injection/biopsy site
2. Bleeding at injection/biopsy which may occur around the kidney or anywhere along the needle track and which may be sufficient to entail clinically significant anemia, acute kidney injury (AKI), hematoma, and in the case of subcapsular bleeding, a “Page kidney” and acute hypertension
3. Surgical damage to the kidney from needle injury, as well as injuries of other structures that include connective tissues, bone, and intra-abdominal viscera.

1.5.1. Potential Risks Associated with Renal Biopsy

Autologous kidney cells will be obtained from individual subjects via a kidney biopsy performed according to standard medical practice [1-3] and consistent with standard operating procedures at participating hospitals/medical institutions. A minimum of 2 tissue cores from a single kidney biopsy is needed to obtain sufficient renal cortical tissue for the production of REACT. A 16-gauge biopsy needle measuring approximately 10 mm in length will remove 0.01-0.02% of the average total volume of the diseased kidney. Since approximately 0.001% of the total number of renal glomeruli will be harvested,[3] the biopsy is not expected to adversely affect kidney function.

Kidney biopsies for diagnostic procedures are of low risk and often conducted under sedation on an outpatient basis in the US.[30, 31] When performed by qualified interventional physicians, a renal biopsy properly targeted towards the cortex produces limited renal damage.[27] On the other hand, reports of kidney damage at the biopsy site describe vascular injury and varying degrees of ischemia and infarction. The severity of damage depends on the size and number of vessels injured during the biopsy procedure.[32]

Hemorrhage is the most common adverse event associated with a routine kidney biopsy. Nearly all patients experience microscopic hematuria as a result of the biopsy, but this is not clinically significant.[2, 31] On the other hand, gross hematuria occurs in 3-9% of patients,[30, 31] and generally resolves by 24 hr post-biopsy. The most serious complication is severe bleeding that requires transfusion and/or results in patient death. Transfusions are needed in less than 1% of renal biopsies, and death occurs in less than 0.01% of cases.[33-35]

1.5.2. Potential Risks Associated with REACT Product

The investigational product, REACT, is composed of autologous renal cells obtained from the same subject via kidney biopsy. Based on experience with autologous stem cell transplantation, the risk of an immune response (e.g., graft rejection) caused by REACT injection into the kidney seems unlikely.

Since the kidney is a highly perfused organ, it is doubtful that the injected SRC will remain localized at the injection site. The three locations considered to be the most likely destinations of migrated SRC are: 1) the sub-capsular space; 2) the systemic circulation; and, 3) the urinary tract. Leakage of SRC into the sub-capsular space is not expected to pose a risk to the subject. For example, the sub-capsular space is commonly used to inject endocrine tissue, such as islet cells.[36] The renal capsule also serves as a niche for native stem cells capable of migrating into the renal parenchyma.[37] Additionally, direct injection into the kidney mitigates the possible entry of SRC into the systemic circulation by providing a natural route of elimination via the urinary tract. Furthermore, intravenous administration of heterologous, allogeneic stem cells (ie, mesenchymal stem cells) has been evaluated in clinical trials and yielded no significant risk to subjects.[24-28]

Porcine Skin Type B gelatin used in the formulation of REACT meets Pharmaceutical and Edible Gelatin Monograph (European Pharmacopeia 7.0, US Pharmacopeia-National Formulary USP35 NF30) requirements. Gelatin is widely used in pharmaceutical and medical applications, including cellular transplantation for regenerative products. Gelatin would not be expected to cause adverse effects in study subjects based on its biocompatible nature, widespread use, and results of GLP toxicology studies with REACT-containing porcine gelatin.

1.5.3. Potential Risks Associated with REACT Treatment

A percutaneous technique is used to access the kidney for REACT delivery. The percutaneous approach has been used for over a decade in ablation of renal masses. A concise review of this method can be found in Salagierski and Salagierski (2010).[38] Safety measures will be executed during REACT treatment and post-surgical follow-up to reduce the potential for excessive bleeding and other adverse events. Patients will be closely monitored as discussed in Section 6.

Cain and coworkers (1976) [39] reported that renal cell homogenates injected into rodent kidneys produced no significant adverse events. Similarly, the observed morphological effects following REACT delivery into the kidney were consistent with those reported for repeated kidney biopsies taken from canines, ie, the presence of a mature connective tissue track with no functional deficits linked to minimal structural changes.[32] Increasing intracapsular kidney water volume in canines can elevate intra-kidney pressure as well as transient increases in kidney weight and systemic blood pressure.[19, 20] However, in the pilot canine studies that assessed the short-term effects of volume administration on blood pressure, there were no adverse effects on blood pressure following volume escalation up to 6 mL per kidney of REACT.

1.6. Potential Benefits

The potential to achieve clinically significant improvement in CKD is supported by studies that tested REACT in pre-clinical animal models of kidney insufficiency, i.e., surgical models for decreased kidney function in otherwise healthy rats and dogs plus the ZSF-1 rat model of T2DM. The main finding was that REACT significantly decreased the rate of structural and functional deterioration in already compromised kidneys to an extent that was clinically relevant in the animal model. Therefore, the potential exists for subjects participating in this clinical trial to realize therapeutic benefit from REACT treatment, such as a possible reduction in the rate of progression of CKD.

### 2. Phase I Trial Objectives and Purpose

This clinical trial relates to a regenerative cell-based product, -Kidney Augment (REACT), with the aim of improving renal function in subjects who have CKD and T2DM. Therapeutic intervention with REACT is intended to delay the need for renal replacement therapy (dialysis or transplant) which, based on the current standard-of-care, is inevitable for patients with end-stage CKD. The purpose of the present study is to compare the safety and efficacy of up to 2 injections of REACT given 3 months (+12 weeks) apart (maximum) in subjects who are randomized to receive their first treatment as soon as the REACT product is made available versus subjects who are randomized to undergo contemporaus, standard-of-care treatment for CKD during the first 12-18 months prior to receiving up to 2 injections of REACT. In addition, each subject's annual rate of renal decline, based on adequate historical, clinical data from 18 months prior to the Screening Visit, serves as a comparator to monitor the rate of progression of renal insufficiency pre- and post-REACT injection.

REACT treatment reduces the rate (slope) of eGFR decline and improves renal function over the 24 month period following the last REACT injection.

2.1. Primary Objective

To assess the safety of REACT injected in one recipient kidney.

### Primary Endpoints:

Change in eGFR through 6 months following two REACT injections
Incidence of renal-specific procedure and/or product related adverse events (AEs) through 6 months post-injection

2.2. Secondary Objective

To assess the safety and tolerability of REACT administration by assessing renal-specific adverse events over a 24 month period following injection.

Secondary Endpoint Renal-specific laboratory assessments through 24 months post-injection.

2.3. Exploratory Objective

To assess the impact of REACT on renal function over a 24 month period following injection.

### Exploratory Endpoints:

Clinical diagnostic and laboratory assessments of renal structure and function (including eGFR, serum creatinine, and proteinuria) to assess changes in the rate of progression of renal disease.
Vitamin D levels
Iohexol imaging
Blood pressure control
MRI assessment of kidney volume

### 3. Investigational Product

3.1. Description of Study Drug

REACT is an injectable product composed of SRC formulated in a biomaterial (gelatin-based hydrogel). Table 9 presents an overview of the investigational product. Refer to the Investigator's Brochure for a detailed description of SRC and REACT as well as the manufacturing process.

TABLE 9
Investigational Product

| | Investigational Product |
|---|---|
| Product Name: | REACT (-Kidney Augment) |
| Dosage Form: | Renal cells obtained from autologous kidney biopsy |
| | tissue are expanded and SRC selected. SRC are |
| | formulated in a gelatin-based hydrogel at a |
| | concentration of 100 × 10⁶ cells/mL. This sterile cell |
| | preparation (REACT) is contained in a sterile |
| | 10 mL syringe and shipped to the clinical site for use. |
| Unit Dose | The dose of REACT is adjusted to 3 × 10⁶ cells/g |
| | estimated kidney weight determined from MRI study. |
| Route of | Percutaneous injection into the cortex of the biopsied |
| Administration | kidney |
| Physical | Sterile, labelled, 10 mL syringe containing up to 8 mL |
| Description | of REACT |
| Manufacturer | Twin City Bio LLC, Winston-Salem, North Carolina, |
| | USA |

3.2. Procurement and Manufacture of REACT

REACT is manufactured in a GMP facility at Twin City Bio LLC located in in Winston-Salem, N.C., USA.

3.2.1. Biopsy

The biopsy material is collected using standard surgical techniques to assess the left or right kidney. A minimum of 2 tissue cores each measuring in 1.5 cm must be collected using a 16 gauge biopsy needle to provide sufficient material for the manufacture of autologous REACT. When the biopsy material is received at Twin City Bio LLC, the samples are labeled and strict documentation measures followed to insure that product traceability is maintained. Twin City Bio LLC notifies the site concerning the adequacy and quality of the biopsy sample for the manufacture of REACT, and confirms the scheduled date for REACT injection. If the biopsy material cannot be used, the subject should be discontinued from the study.

3.2.2. Selection of SRC

Approximately 4 weeks prior to the subject's planned REACT treatment, autologous renal cells are removed from the vapor phase of a liquid nitrogen freezer, thawed, and isolated from kidney tissue by enzymatic digestion. Cells are cultured and expanded using standard techniques.

The cell culture medium is designed to expand primary renal cells and does not contain any differentiation factors. Harvested renal cells are subjected to density gradient separation to obtain SRC, which are composed primarily of renal epithelial cells known for their regenerative potential.[40] Other parenchymal (vascular) and stromal (collecting duct) cells may be sparsely present in the autologous SRC population.

If sufficient cells are available, the same biopsy material is used to make additional REACT preparations for research studies and stored, under GMP conditions, in the vapor phase of a liquid nitrogen freezer.

All subjects receive a series of 2 REACT injections. The time and events table shows that the series of 2 REACT injections are administered 3 months apart with a study visit window of 12 weeks. Regardless, every attempt is made to ensure that the second REACT injection is administered 3 months after the first injection. Twin City Bio LLC notifies the site to obtain information about the scheduled date for the second injection.

3.2.3. Formulation

SRC is formulated in a gelatin-based hydrogel to improve stability during transport and delivery upon injection into the renal cortex. Porcine gelatin is dissolved in buffer to form the thermally responsive hydrogel. Although fluid at room temperature, this biomaterial gels when cooled to refrigerated temperature (2 to 8° C.). Prior to injection, the REACT investigational product must be warmed to ≥20 C up to 26 C to liquefy the hydrogel.

3.2.4. REACT Product for Injection

Ten to 14 days prior to the scheduled date for REACT injection, the subject reports to the clinic and undergoes assessments to verify continued eligibility. If the subject does not qualify for REACT injection, the Investigator and the Sponsor discusses possible options, for example, if there is sufficient stability to attempt an REACT injection at a future date. If the subject still qualifies, the REACT product is manufactured and shipped to the clinical center. It is the responsibility of the site to ensure REACT shipments are delivered directly to site personnel. REACT is injected into the biopsied kidney of eligible subjects using a percutaneous approach. The percutaneous method employs a standardized technique (such as that utilized in the ablation of renal masses by radiofrequency or cryogenic methods).[38]

Two REACT injections are planned for each subject. However, if there appears to be any untoward safety risk, or rapid deterioration of renal function, or development of uncontrolled diabetes or uncontrolled hypertension, or development of a malignancy or an intercurrent, then the second REACT injection should not be administered.

Renal cells that may have been frozen but not used to manufacture REACT remain in the vapor phase of a liquid nitrogen freezer at Twin City Bio LLC until the EOS Visit. At that time, if these renal cells are no longer needed, they are de-identified of all personal information and stored in the vapor phase of a liquid nitrogen freezer for a maximum of 5 years. The aim is to test these renal cells in laboratory research studies. During the informed consent process, each subject provides written consent for the storage and future use of autologous cells not used for REACT injection. Subjects have the option of having these cells destroyed upon study completion.

3.2.5. REACT Dose

The dose of REACT for subjects in the Phase 1 clinical trials (TNG-CL010 and TNG-CL011) was 3×10⁶ SRC/g estimated kidney weight (g KWest). Similarly, in the present study, each REACT injection contains 3×10⁶ cells/g KWest. Since the concentration of SRC is 100×10⁶ cells/mL of REACT, the dosing volume is 3.0 mL for each 100 g of kidney weight. The volume of REACT to be administered is determined by pre-procedure MRI volumetric 3D evaluation or ellipsoid formula (Length×width AP plane×width Transverse plan×0.62). Examples of dosing volumes based on estimated kidney weight are shown in Table 10.

TABLE 10
REACT Dosing Relative to Estimated Kidney Weight

| | | REACT | SRC Delivered |
|---|---|---|---|
| Estimated Kidney Weight (gKW^{est})^{a,b} | | Dosing | (Number of |
| Median Weight (g) | Weight Range (g) | Volume (mL) | Cells × [symbol] |
| 100 | 95-108 | 3.0 | 300 |
| 117 | 109-125 | 3.5 | 350 |
| 133 | 126-141 | 4.0 | 400 |
| 150 | 142-158 | 4.5 | 450 |
| 167 | 159-175 | 5.0 | 500 |
| 183 | 176-191 | 5.5 | 550 |
| 200 | 192-208 | 6.0 | 600 |
| 217 | 209-225 | 6.5 | 650 |
| 233 | 226-241 | 7.0 | 700 |
| 250 | 242-258 | 7.5 | 750 |
| — | >259 | 8.0^{c} | 800 |

Abbreviations: Estimated Kidney Weight (gKW^{est}); SRC (Selected Renal Cells).
Notes:
^{a}The dose of REACT will be 3 × 10⁶ cells/g estimated kidney weight.
^{b}Kidney weight will be estimated from an MRI study performed before renal biopsy.
^{c}8 mL will be the maximum dosing volume (mL). [symbol]
indicates data missing or illegible when filed

The dose of REACT is based on kidney volume calculated via MRI. In contrast to other methods, measurements of renal volume using MRI are more accurate, and acquire true tomographic data along any orientation without the risk of ionizing radiation or nephrotoxic contrast agents. Renal volume measurements (mL) estimated from MRI are approximately 92 to 97% of dry weight measurements in grams for isolated organs trimmed of perirenal fat. As a conservative approach, the REACT dose is calculated using a conversion of one g equals one mL. The volume of REACT to be administered is determined by pre-procedure MRI volumetric 3D evaluation or ellipsoid formula (Length×width AP plane×width Transverse plan×0.62). This ensures that subjects do not receive REACT doses higher than those previously tested in animal studies.

3.2.5.1. Rationale for Two REACT Injections

All subjects are intended to receive two planned REACT injections to allow dose-finding and evaluate the duration of effects. The scientific rationale, based on non-clinical studies, is that the biologically active component of REACT (homologous, autologous, SRC) delays progression of experimental models of CKD by augmenting renal structure and function.[7-12] As a result, the more cells that can be infused, the greater the potential improvement in renal function. The total number of cells that can be delivered into a kidney at one time is limited by the size of the kidney, however, as well as the inelasticity of the renal capsule. Consequently, it may be possible to improve therapeutic benefit by administering greater numbers of SRC via a second injection, given after cells from the first injection have become incorporated into the kidney.

Apart from increasing SRC numbers by administering 2 REACT injections into the same kidney, the duration of effects can be evaluated. The processes by which functional nephrons become disabled in kidneys with CKD may, over time, adversely affect “new” cells delivered via REACT injection. Consequently, REACT might not result in long-term, therapeutic benefit. Exploring the effects from a second REACT injection, given at an appropriate interval after the first injection, would address this question.

In the present study, subjects are administered a second REACT injection 3 months after the first injection, with a study visit window of 12 weeks. Regardless, every attempt should be made to ensure that the second REACT injection is administered 3 months after the first injection. However, if there appears to be any untoward safety risk, or rapid deterioration of renal function, or development of uncontrolled diabetes or uncontrolled hypertension, or development of a malignancy or an intercurrent infection, then the second REACT injection is not administered.

3.2.5.2. Safety of Two REACT Injections

To assess the safety of administering two doses of REACT into the biopsied kidney, a canine GLP toxicology study was conducted (Refer to Section 1.2.2). Similar to the clinical study design, study animals (n=8) underwent renal biopsies at 4 to 6 weeks prior to baseline. Each dose was delivered into both kidneys at baseline and 3 months; animals were observed for 6 months following the baseline injection. While control animals received PBS, REACT-treated animals received a two-fold greater dose than that used in the present clinical study. Briefly, no detrimental effects of two doses of REACT into the biopsied kidney were observed in comparison to control animals 6 months after baseline treatment. Pathological assessment showed no REACT safety-related (macroscopic or microscopic) findings in either the target organ (kidney) or non-target organs examined. No treatment-related kidney findings were noted following enhanced evaluation of 8 areas of each kidney (3 stains per area), including assessment and scoring of 150 glomeruli per kidney. All kidneys appeared normal, apart from changes related to injection site scars. There were no signs of renal insufficiency, and no indications of decreased GFR. Detailed information is provided in the Investigator's Brochure.

3.3. Study Drug Packaging

The product delivery system consists of 3 components:

1) 10 mL standard, Luer-Lok® syringe

2) Package for containment of the syringe

3) REACT shipping container for transportation of the package to the clinical site

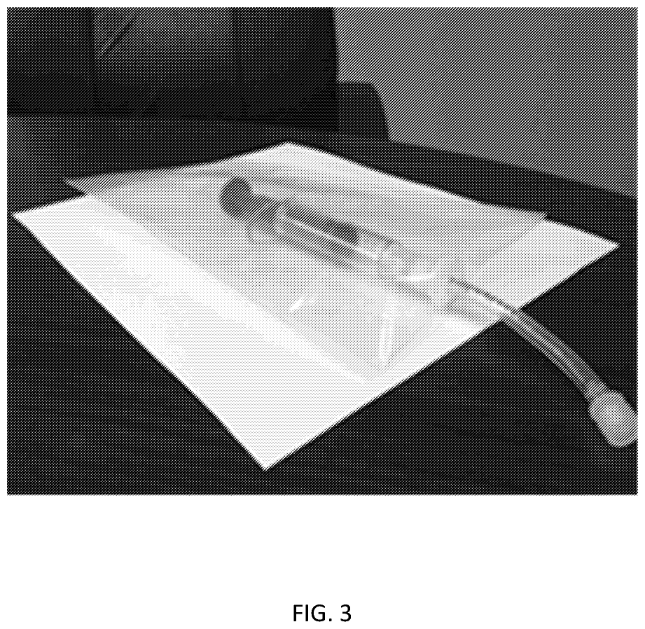

The syringe containing REACT is shipped to the clinical site encased in a package designed to maintain integrity of the product as well as sterility of the product and syringe. A representative image of the product delivery system is shown in FIG. 3.

The product delivery system is made from components listed in Table 11. Materials that come into contact with the REACT product are USP class VI or equivalent. The syringe, tubing and ancillary parts are obtained from vendors listed in Table 11 or other vendors that satisfy the biocompatibility classification and product compatibility testing requirements. The syringe is pre-sterilized in the package by gamma sterilization. After filling, the tubing is sealed and cut.

TABLE 11
Product Delivery System Components

| | | Production | REACT | Biocompatibility |
|---|---|---|---|---|
| Components | Vendor | Material | Contact | Test Reference* |
| Syringe | Merit | Polycarbonate, | Direct | ISO 10993 |
| | Medical or | Silicone or | | USP Class VI |
| | Becton- | Polypropylene, | | |
| | Dickinson | Silicone | | |
| Tubing | Saint- | Polyvinyl | Direct | Ph Eur 3.1.1.2 |
| | Gobain | Chloride | | ISO 10993 |
| | Performance | | | USP Class VI |
| | Plastics | | | |
| Luer-Lok ® | PAW | Polyethylene | Direct | USP Class VI |
| Fittings | BioScience | | | |
| | Value | Polypropylene | Direct | USP Class VI |
| | Plastics | MABS | | |

*Additional testing has been performed (Cytotoxicity, MEM Elution, in vitro: USP <87>; Rabbit Blood Cell Hemolysis: ASTM F756-00; Physicochemical Test for Plastics: USP <661>).

3.4. Study Drug Label

The REACT product is made from expanded autologous SRC obtained from each individual subject's kidney biopsy and is, therefore, subject-specific. Each package containing the syringe has affixed to it a label containing the following: “FOR AUTOLOGOUS USE ONLY”. In addition, the label indicates that this drug (REACT) is for “Investigational Use ONLY”.

3.5. Study Drug Transportation

All biopsy specimens are transported to Twin City Bio LLC using packaging mandated in the Code of Federal Regulations (42 CFR Part 72) and according to individual carrier guidelines.

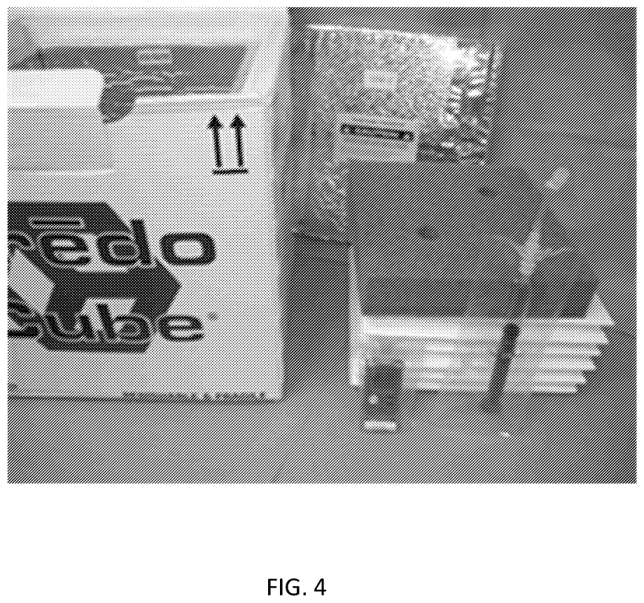

Because the REACT hydrogel formulation must maintain a temperature from 2 to 8° C. during shipping, REACT product is transported from Twin City Bio LLC to the clinical site in a shipping container validated to maintain temperature at 2 to 8° C. The REACT package is placed in a plastic outer containment bag and then in a refrigerated shipping container from Minnesota Thermal Sciences. A temperature recorder is also included in the shipping container. A representative image of the shipping container is shown in FIG. 4.

When the shipping container arrives at the clinic in time for a scheduled injection, the inner REACT package is removed from the shipping container and equilibrated to controlled room temperature (≥20° C. up to 26° C.).

Two individuals independently verify identifying information in the presence of the subject, thereby confirming that the information is correctly matched to the specific study participant.

Once the hydrogel becomes liquid, the surgical assistant opens the container in a sterile field, and transfers the syringe to the physician who performs the percutaneous injection of REACT into the renal cortex of the biopsied kidney.

3.5.1. Disposition of Stored Specimens

Specimens are stored in the vapor phase of a liquid nitrogen freezer.

### 4. Investigational Plan

4.1. Overall Study Design

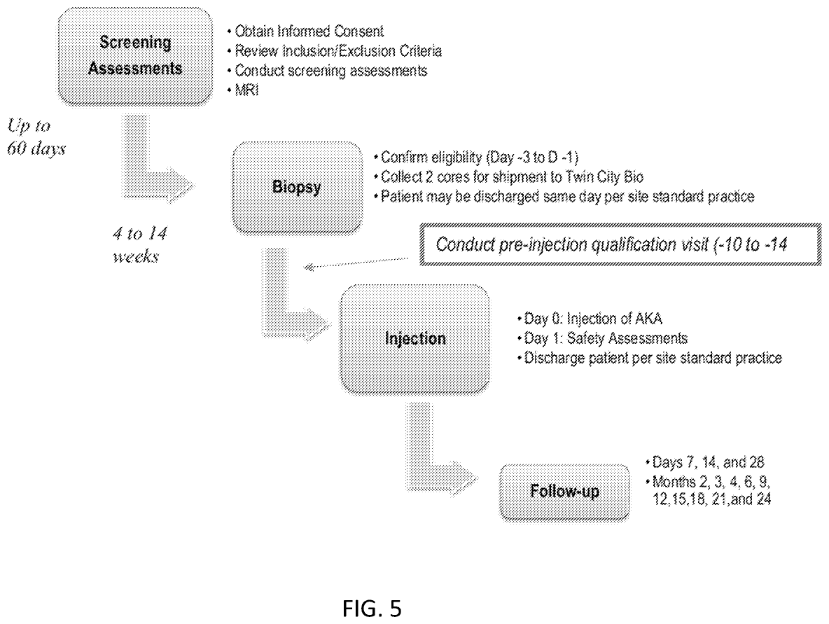

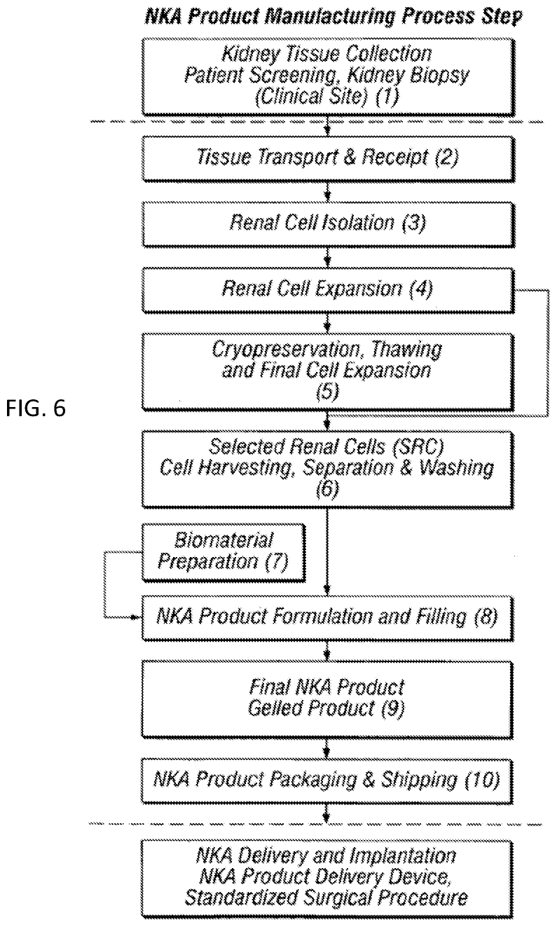

An overview of the study flow is shown in the diagram in FIG. 5.

After patients have signed the ICF, they are screened for entry into the study. Screening assessments include laboratory assessments, physical examination, and an ECG and MRI study, all of which are performed before the biopsy is taken. A biopsy of the left kidney is taken from patients who meet all I/E criteria within 45 days of the first screening assessment. During the biopsy procedure, two tissue cores are collected and sent to Twin City Bio for manufacture of REACT. If the patient experiences significant AEs/SAEs following biopsy (e.g., excessive bleeding, development of AV fistula) that precludes safe injection, then the patient is discontinued from the study.

One-two weeks after receipt at Twin City Bio's GMP facility in North Carolina, USA, Twin City Bio notifies the site if the tissue received was of sufficient size and quality for manufacture of REACT. If results are positive, the site confirms the scheduled date of injection. If the biopsy is not able to be used for manufacture of REACT (for whatever reason), the patient is discontinued from the study.

Ten to 14 days before the scheduled injection, the patient reports to the clinic for a pre-injection qualification visit including final review of I/E criteria and a renal scintigraphy study. The site notifies Twin City Bio to manufacture REACT product from frozen renal cells if the patient is eligible for injection. On the day of injection (Day 0), the patient arrives at the hospital and receives an REACT injection into the kidney that was biopsied.

4.2. Number of Subjects

Up to 15 subjects who complete screening procedures and satisfy all inclusion and exclusion criteria are enrolled.

4.3. Treatment Compliance

Eligible subjects receive their autologous REACT preparations via a series of up to two injections. The investigational product is administered into the biopsied kidney using a percutaneous approach. REACT product preparation and dosing procedures are specified in this protocol as well as the Study Reference Manual.

All subjects are intended to receive two REACT injections. If there appears to be any untoward safety risk, or if the subject's health status would be jeopardized, then the second REACT injection is not administered.

4.4. Study Duration

Subjects begin their series of REACT injection(s) as soon as the autologous REACT preparation is made available. With a one-month interval prior to the first REACT injection, and a 3-month interval before the second injection, plus a 24-month follow-up period after the final injection, the study duration is 28 months for a series of 2 REACT injections

### 5. Study Population

5.1. Subject Inclusion Criteria

Unless otherwise noted, subjects must satisfy each inclusion criterion to participate in the study. Inclusion criteria are assessed at the Screening Visit, prior to renal biopsy, and before each REACT injection unless otherwise specified.
1. The patient is male or female, 18 to 65 years of age on the date of informed consent.
2. The patient has a documented history of abnormality of the kidney and/or urinary tract in addition to documented history of CAKUT.
3. The patient has an established diagnosis of Stage III/IV CKD not requiring renal dialysis, defined as having an eGFR between 14 and 50 mL/min/1.73 m2 inclusive at the Screening Visit prior to REACT injection.
4. The subject has blood pressure less than 140/90 at the Screening Visit, prior to renal biopsy, and prior to REACT injection(s). Note BP should not be significantly below 115/70.
5. A minimum of three measurements of eGFR or sCr are obtained at least 3 months apart prior to the Screening Visit and within the previous 24 months to define the rate of progression of CKD.
6. The patient is willing and able to refrain from NSAID consumption (including aspirin) as well as clopidogrel, prasugrel, or other platelet inhibitors during the period beginning 7 days before through 7 days after both the renal biopsy and REACT injection(s).
7. The patient is willing and able to refrain from consumption of fish oil and platelet aggregation inhibitors, such as dipryridamole (i.e., Persantine®), during the period beginning 7 days before through 7 days after both the renal biopsy and REACT injection(s).
8. The patient is willing and able to cooperate with all aspects of the protocol.
9. The patient is willing and able to provide signed informed consent.

5.2. Subject Exclusion Criteria

Subjects who satisfy any exclusion criterion listed below are not eligible to participate in the study. Exclusion criteria is assessed at the Screening Visit, before renal biopsy, and before each REACT injection unless otherwise noted.
1. The patient has a history of renal transplantation.
2. The patient has a diagnosis of hydronephrosis, SFU Grade 4 or 5.
3. The patient has an uncorrected VUR Grade 5.
4. The patient's cortical thickness measures less than 5 mm on MRI
5. The patient has a known allergy or contraindication(s), or has experienced severe systemic reaction(s) to kanamycin or structurally similar aminoglycoside antibiotic(s)
6. The patient has a history of anaphylactic or severe systemic reaction(s) or contraindication(s) to human blood products or materials of animal origin (e.g., bovine, porcine).
7. The patient has a history of severe systemic reaction(s) or any contraindication to local anesthetics or sedatives.
8. The patient has a clinically significant infection requiring parenteral antibiotics within 6 weeks of REACT injection.
9. The patient has acute kidney injury or has experienced a rapid decline in renal function during the last 3 months prior to REACT injection.
10. The patient has any of the following conditions prior to REACT injection: renal tumors, polycystic kidney disease, anatomic abnormalities that would interfere with the REACT injection procedure or evidence of a urinary tract infection.
Note: anatomic abnormalities are not exclusionary if kidney remains accessible and meets the criteria to receive REACT injection
11. The patient has class III or IV heart failure (NYHA Functional Classification)
12. The patient has FEV1/FVC ≥70%.
13. The patient has a history of cancer within the past 3 years (excluding non-melanoma skin cancer and carcinoma in situ of the cervix).
14. The patient has clinically significant hepatic disease (ALT or AST greater than 3 times the upper limit of normal) as assessed at the Screening Visit.
15. The patient is positive for active infection with Hepatitis B Virus (HBV), or Hepatitis C Virus (HCV), and/or Human Immunodeficiency Virus (HIV) as assessed at the Screening Visit.
16. The patient has a history of active tuberculosis (TB) requiring treatment within the past 3 years.
17. The patient is immunocompromised or is receiving immunosuppressive agents, including individuals treated for chronic glomerulonephritis within 3 months of REACT injection.
Note: inhaled corticosteroids and chronic low-dose corticosteroids (less than or equal to 7.5 mg per day) are permitted as are brief pulsed corticosteroids for intermittent symptoms (e.g., asthma).
18. The patient has a life expectancy less than 2 years.
19. The female patient is pregnant, lactating (breast feeding), or planning a pregnancy during the course of the study. Or, the female patient is of child-bearing potential and is not using a highly effective method(s) of birth control, including sexual abstinence. Or, the female patient is unwilling to continue using a highly-effective method of birth control throughout the duration of the study.
20. The patient has a history of active alcohol and/or drug abuse that would impair the patient's ability to comply with the protocol.
21. The patient's health status would be jeopardized by participating in the study.
22. The patient has used an investigational product within 3 months prior to REACT injection.

5.3. Prohibited and Concomitant Medications
The consumption of NSAIDs (including aspirin) as well as clopidogrel, prasugrel, or other platelet inhibitors is prohibited during the study beginning 7 days before through 7 days after both the renal biopsy and REACT injection(s).
Aspirin, up to a dose of 100 g/day, is accepted for primary prevention of heart disease in subjects with diabetes who are greater than 40 years of age or have additional risk factors for cardiovascular disease or stroke, and for whom the perceived benefits of aspirin therapy outweigh the risks associated with treatment.
Intake of fish oil and platelet aggregation inhibitors, such as dipryridamole (ie, Persantine), is prohibited during the study beginning 7 days before through 7 days after both the renal biopsy and REACT injection(s).
Subjects who are undergoing treatment with an ACEI or an ARB must have initiated therapy at least 8 weeks prior to renal biopsy. Treatment must be stable during the 6-week period immediately prior to REACT injection. Stable treatment is defined as dose adjustment no less than one-half of the current dosage and no more than 2 times the current dosage. In addition, except where medically necessary, no changes should be made to the ACEI or ARB dosing regimen from Screening through the 12-month EOS Visit. Dose interruptions up to 7 days due to medical necessity are allowed.
Medications that interfere with measurements of sCr should be avoided during the study, such as trimethoprim, dronedarone, and cimetidine. If such medications are required based on medical necessity, then the circumstance should be discussed with the Medical Monitor and documented within the CRF.
Use of investigational drugs is prohibited during the course of the study. Investigational drugs are defined as drugs that have not been approved for use by the FDA.

5.4. Subject Withdrawal

If a subject withdraws from the study before having the renal biopsy, the subject is considered a screen failure. If a subject withdraws from the study following the renal biopsy but before the first REACT injection, the subject is not be a screen failure, but is not considered enrolled and may be replaced. If a subject withdraws from the study after REACT injection but before the end of the follow-up period, the subject cannot be replaced.

Every effort should be made to ensure that subjects who have been injected with AKA return for all subsequent follow-up visits and procedures, including the EOS Visit.

### 6. Study Visits

The schedules of clinical assessments and procedures to be performed during the study are displayed in the time and events tables, i.e., Table 1. Similarly, the schedules of sample collection and clinical laboratory evaluations planned for the study are displayed in the laboratory time and events tables, i.e. Table 2. Before conducting any study specific assessments or procedures (including screening), the subject provides written informed consent in accordance with ICH GCP guidelines and 21 CFR Part 50.

6.1 Screening

All screening assessments take place in a timeframe that allows for scheduling of the renal biopsy within 60 days of the Screening Visit. For example, if a subject signs the consent form and then goes to the laboratory for his/her blood draw two days later, then the date of the blood draw is considered the date of the first screening assessment (i.e., not the date that the consent form was signed).

Renal ultrasound is performed at the Screening Visit to verify subject eligibility (i.e., no evidence of renal tumors, polycystic kidney disease, renal cysts or other anatomic abnormalities that would interfere with the REACT injection procedure) along with obtaining a baseline echogenicity reading. Additionally, a MRI study without contrast is performed from the time of Screening Visit through Day −1 before renal biopsy to determine kidney size and volume.

To qualify for study enrollment, the subject's eGFR must be between 15 and 50 mL/min/1.73 m² inclusive at the Screening Visit. To define the eGFR for entry criteria, the site uses the eGFR assessed during screening and calculate using the CKD-EPI equation.[41]

6.2. Biopsy

The biopsy is scheduled within 60 days of the Screening Visit. The biopsy takes place in a time frame that allows for scheduling of the first REACT injection approximately one month later. Renal biopsy cores are typically collected on a Wednesday or Thursday.

Subjects report to the hospital or clinical research center one to three days before the biopsy for pre-biopsy assessments. As much as possible, pre-biopsy laboratory samples are collected and assessed to verify continued eligibility. After admission and final verification of inclusion and exclusion criteria, the biopsy is performed as described in Section 7.5.1.

A minimum of 2 biopsy cores measuring 1.5 cm in length a piece and collected using a 16 gauge biopsy needle under sterile conditions from each enrolled subject are sent to Twin City Bio LLC using a refrigerated shipping container. Twin City Bio LLC contacts the site to confirm receipt of sufficient biopsy material to manufacture REACT. If the biopsy cannot be used to manufacture REACT, the subject is discontinued from the study.

Subjects who do not experience complications from the biopsy are discharged the same day consistent with site standard practice. Otherwise, the subject remains in the hospital overnight for observation. The subject is discharged on the day after the biopsy so long as any biopsy-related AEs have resolved, stabilized, or returned to baseline.

6.3. REACT Injection

Subjects receive two planned REACT injections to allow dose-finding and to evaluate the duration of effects. As indicated on the time and events tables (i.e., Table 1), the series of 2 REACT injections is administered 3 months apart with a study visit window of 12 weeks. Regardless, every attempt is made to ensure that the second REACT injection is administered 3 months after the first injection.

If there appears to be any untoward safety risk, or rapid deterioration of renal function, or the development of uncontrolled diabetes or uncontrolled hypertension, or the development a malignancy or an intercurrent infection, then the second REACT injection is not be administered.

Eligible subjects arrive at the hospital or clinical research center on the morning of REACT treatment. The subject is injected with autologous REACT using a percutaneous approach as discussed in Section 7.5.2.

6.4. Discharge After REACT Injection

On the day after REACT injection and prior to discharge, an ultrasound is performed to detect possible, subclinical adverse effects (e.g., swelling, fluid accumulation). If product- or procedure-related AE's occurred following REACT injection, the subject is discharged until the AE's have resolved, stabilized, or returned to baseline. If consistent with the site's standard practice, the subject is discharged the same day as the REACT injection after no less than 2 hours of observation and monitoring.

6.5. Follow-Up Visits

The subject returns to the clinic for follow-up visits on Days 7, 14, and 28 (±3 days) and Month 2 (±7 days) after the first and second REACT injections. When the series of 2 REACT injections are administered 3 months apart, the subject does not attend follow-up visits scheduled at 3 and 6 months. These visits are shown as “optional” on the time and events tables (Table 1) as well as the laboratory time and events tables (Table 2). Instead, the subject reports to the clinical center 10 to 14 days before the planned, final REACT injection to undergo pre-treatment assessments.

Following the final REACT injection, subjects complete long-term, follow-up assessments of safety and efficacy through Months 6, 9, 12, 15, 18, 21, and 24 (±7 days) post-treatment.

6.6. End-of-Study Visit

This section describes situations in which a subject undergoes the EOS visit; for example, due to premature discontinuation from the study or completion of all protocol-specified follow-up visits.
If a subject discontinues from the study after undergoing the renal biopsy but before REACT injection, then that subject completes all EOS assessments except for the MRI and/or renal scintigraphy studies. If the subject is experiencing an investigational product- or study procedure-related SAE, then the subject is not discontinued until the SAE has resolved, stabilized, or returned to baseline.
If a subject discontinues from the study after undergoing one or two REACT injections but before completing all of the protocol-specified follow-up visits, then he/she has the EOS Visit at the time of discontinuation. If the subject is experiencing an investigational product- or study procedure-related SAE, then the subject is not discontinued until the SAE has resolved, stabilized, or returned to baseline.
If a subject completes all of the protocol-specified follow-up visits, he/she undergoes all EOS assessments 24 months after the final REACT injection at the EOS Visit. If the subject is experiencing an investigational product- or study procedure-related SAE, then the subject is not discontinued until the SAE has resolved, stabilized, or returned to baseline.

6.7. Study Completion

Completion of the study is defined as the time when the last subject completes the EOS Visit, or when the last subject is considered lost to follow-up, withdraws consent, or dies.

### 7. Study Assessments and Procedures

7.1. Demography and Medical History

Demographics characteristics are obtained for each subject at the Screening Visit.

All CKD-related medical history and all other significant medical history is recorded in the CRF beginning at the Screening Visit. Throughout the study, medical conditions that are still ongoing are regularly updated in the CRF.

7.2. Clinical Evaluations

7.2.1. Vital Signs

Vital signs to be measured include systolic/diastolic blood pressure, heart rate, respiration rate, and temperature. Blood pressure is measured after the subject has rested in a sitting position for a minimum of 5 minutes. At the Pre-Biopsy Visit (Day −3 to Day−1) and Pre-Injection Visit (Day −14 to Day −10 Visit), three BP measurements are taken and the average of the 3 measurements (for systolic and diastolic pressure) used to satisfy entry criteria and entered into the CRF.

7.2.2. Physical Examination

The comprehensive examination assesses all pertinent body systems while the interim examination includes specific assessments of those body systems deemed appropriate for that subject. As a general rule for the interim examination, the subject's adverse events are reviewed prior to, or in conjunction with, the examination and include assessment of related body systems as appropriate. Only clinically significant abnormalities are recorded in the CRF.

The subject's weight is measured at every visit that includes a complete or interim physical examination. Body Mass Index (BMI) will be calculated as kg/m².

7.2.3. ECG

A 12-lead ECG is obtained after the subject rests on his or her back for 5 minutes with the blood pressure cuff applied but not inflated at the level of the heart. ECG recordings are assessed and the results entered into the CRF.

7.2.4. Concomitant Medications

Concomitant medications are recorded in the CRF as follows:
Screening Visit until first REACT injection: Record any CKD-specific medications as well as medications that may affect renal hemodynamics and/or serum creatinine measurements. In addition, record any medications used to treat an AE that is documented in the CRF. Surgical medications used during the biopsy procedure do not need to be captured in the CRF unless their use falls outside of expected dosages and/or frequencies of administration.
First REACT injection until 3 to 6 months of follow-up: Record any medications taken until 3 to 6 months after treatment, depending on when the second REACT injection is administered. Surgical medications used during the REACT injection procedure do not need to be captured in the CRF unless their use falls outside of expected dosages and/or frequencies of administration.
Second (Final) REACT injection until 6 months of follow-up: Record any medications taken until 6 months after the last REACT treatment. Surgical medications used during the REACT injection procedure do not need to be captured in the CRF unless their use falls outside of expected dosages and/or frequencies of administration.
6 months of follow-up through the EOS Visit: Record CKD-specific medications, that may affect renal hemodynamics and medications that may affect serum creatinine measurements. Record medications used to treat any AE documented in the CRF.

7.3. Laboratory Assessments

Planned clinical laboratory evaluations are listed in Table 12. Analyses will be conducted by a central laboratory, except as noted. The scheduled for collecting biological samples during the study are shown in Table 2.

TABLE 12
Clinical Laboratory Evaluations
Clinical Chemistry
Alkaline phosphatase
ALT
AST
ß2-Microglobulin
Bilirubin
Creatinine kinase
FSH (females only)
GGT
HbA_{1c}
LDH
PTH (intact)
Renal Analytes
Albumin
BUN
Calcium
CO₂, total
Creatinine
Cystatin-C
CRP
eGFR (calculated)
Glucose
Phosphorus
Potassium
Sodium
Hematology
Hematocrit
Hemoglobin
RBC count & indices
WBC count & differential
Pregnancy
hCG (serum)-confirmatory
Serology
HBV
HCV
HIV
Coagulation Status
APTT
PT-INR
Platelet count
Lipid Panel
Cholesterol
LDL
HDL
LDL:HDL ratio
Triglycerides
Urinalysis
Albumin
ß2-Microglobulin
Creatinine
Protein
Protein & Albumin:Creatinine Ratio
NGAL
Standard Panel
pH
Ketones
Protein
Blood
Glucose
Pregnancy
Microscopic analysis
Drug Screen
Amphetamine
Barbiturates
benzodiazepines
Cocaine
Opiates
Tetrahydrocannabinol
Phencyclidine

7.3.1. eGFR

GFR is estimated using the Chronic Kidney Disease Epidemiology Collaboration (CKD-EPI) equation that incorporates both serum creatinine and Cystatin C.[41] For comparison to each subject's historical values, it may be necessary to perform a second analysis at the site laboratory used to generate the historical data.

7.3.2. Routine Urinalysis

Urine is collected and analyzed via standard panel. The schedules for collecting each type of urine sample are shown in Table 2.

Urine is collected over two different time periods: 24 hour collection and “spot” urine. Spot urine collections are used for dipstick urinalysis (test stick) assessments. The schedules for collecting each type of urine sample are shown in Table 2. To provide a comprehensive picture of protein and albumin excretion, both total protein and albumin are assessed in all samples.

7.3.3. Hematology

Hemorrhage following REACT injection is a known and foreseeable risk to subjects participating in this study. Therefore, hemoglobin and hematocrit are measured by the sites local laboratory a) before, b) 4 hours after, each REACT injection and compared to baseline levels. Other bleeding parameters (e.g., APTT, PTT-INR, platelets) are also measured.

7.3.4. Virus Serology

The biopsy cores obtained from each subject are used for the expansion and selection of SRC. Contamination with HIV, HBV, and/or HCV would prevent manufacturing of REACT product for that subject. Therefore, each subject undergoes testing for viral blood-borne pathogens, including HIV, HBV, and HCV.

7.3.5. Drug Screen

Consistent with Exclusion Criterion #24 (See Section 5.2), subjects are not eligible to participate in the study if they have an “active history of drug abuse that would impair the subject's ability to comply with the protocol.” Therefore, subjects undergo testing for drugs of abuse.

7.3.6. Pregnancy Screen

A qualitative urine pregnancy test is performed at the site using a test-strip. If the test is positive, then a confirmatory test is performed by the clinical laboratory. If site practices do not accept the results of a test-strip, then a urine sample is sent to the central laboratory for analysis. Post-menopausal women with a confirmatory FSH test do not have to undergo pregnancy testing throughout the study.

7.4. Renal Imaging

7.4.1. Ultrasound

Renal ultrasound is performed at the Screening Visit to verify subject eligibility (i.e., no evidence of renal tumors, polycystic kidney disease, renal cysts or other anatomic abnormalities that would interfere with the REACT injection procedure) along with obtaining a baseline echogenicity reading. Ultrasound is also performed following the in-patient renal biopsy on Day 0 and Day 1, and following the in-patient REACT injection(s) on Day 0 and Day 1 with the aim of monitoring possible, subclinical AEs. Findings from the ultrasound (e.g., resistance index, length, etc.) are recorded on the CRF.

7.4.2. Computerized Tomography

Computerized tomography (CT) may be used in conjunction with ultrasound during the REACT injection procedure, according to the usual standards of care at the investigative site.

7.4.3. Magnetic Resonance Imaging

An MRI study without contrast is performed from the Screening Visit through Day −1 before renal biopsy to determine kidney size and volume. During the site initiation visit, the MRI process is defined for each site, depending on the MRI equipment available. Generally, a 1.5-T unit should be used. MRI imaging studies help determine kidney volume (for dosing calculations). MRI is performed using standard sequences without injection of contrast agents. Renal volume measurements may be calculated, for example, using a fast 3D gradient-echo sequence, VIBE, with an acquisition time of 22 seconds and spatial resolution of 2×1.4×1.2 mm. Imaging parameters are recorded in the source documents and CRF. A total of four MRIs are performed on patients.

7.4.4. Renal Scintigraphy

Renal scintigraphy is used to assess left and right kidney function using the radioactive tracer ^{99m}Tc-dimercaptosuccinic acid (DMSA) or Tc99m MAG3 (Mercaptoacetyl triglycine). This method is considered as the most reliable for measuring renal cortical function. If the site's standard practice is considered sufficiently equivalent to the procedure using ^{99m}Tc-DMSA or Tc99m MAG3, then the site follows its procedure. IA11 patients in this study receive four renal scintigraphy studies. Renal scintigraphy is performed before the first REACT injection, before the last REACT injection, at the 6-Month Visit after the last REACT injection, and at the EOS Visit for all patients.

7.5. Surgical Procedures

7.5.1. Biopsy

Renal biopsy is performed under sterile conditions using an ultrasound- or CT-guided approach consistent with site practices. Two biopsy cores are needed to provide sufficient material for the selection of SRC and manufacture of REACT. Likewise, a 16-gauge needle is used to insure adequate cortical material is obtained. If required, a 15-gauge needle may be used. Bedside examination of the biopsy cores may be performed, if available, to ensure sufficient cortical material has been obtained.

Since the biopsy tissue is used to manufacture REACT, the site ensures that the tissue cores are harvested using sterile conditions so that the risk of contamination during subsequent cell expansion and selection is minimized.

The subject will remain supine for 4 hours with monitoring of hemoglobin, blood pressure, gross hematuria, abdominal/flank pain, and flank ecchymosis. As long as any biopsy-related AEs have resolved, stabilized, or returned to baseline, the subject is discharged from the hospital on the day after the biopsy consistent with site standard practice. Importantly, any pain medication administered after the renal biopsy is selected carefully, avoiding medications with nephrotoxic potential.

If a subject experiences significant adverse events following the biopsy that would put the subject at increased risk for significant adverse effects following REACT injection, then he/she is not treated with REACT but is followed until resolution of the event(s) and then discontinuation from the study.

7.5.2. REACT Injection

Before performing the REACT injection, the operating physician evaluates the subject as follows:
Perform a physical examination to determine the feasibility of the procedure.
Evaluate bleeding parameters, including coagulation panel, PTT-INR, platelets, hemoglobin, hematocrit, and other pertinent laboratory studies.
   Note: Hemorrhage following REACT injection is a known and foreseeable risk to subjects participating in this study. Therefore, hemoglobin and hematocrit are measured a) before, b) 4 hours after, and c) 24 hours after each REACT injection and compared to baseline levels.
Review imaging studies, including ultrasound, MRI, and/or CT, to determine route of access, depth of kidney, and appearance of cortical-medullary junction.
Map potential REACT cell deposition sites.
Determine classification and associated perioperative/post-operative risk according to the American Society of Anesthesiologists (ASA) with respect to airway assessment, medical history, allergies, and medications.
Interview the subject and the subject's family/supporters to discuss the procedure, its risks and possible complications. Answer questions, and obtain written informed consent.

Prophylactic antibiotics are given intravenously according to site standard practice. An initial CT scan may be ordered, if necessary, to evaluate adjacent viscera, renal location, and the presence of renal cysts. In conjunction with ultrasound, a CT scan also may help locate the cortical-medullary junction.

REACT is targeted for injection into the kidney cortex via a needle/cannula and syringe compatible with cell delivery. The intent is to introduce REACT via penetration of the kidney capsule and deposit REACT into multiple sites of the kidney cortex. Initially, the kidney capsule is pierced using a 15- to 20-gauge trocar/access cannula inserted approximately 1 cm into the kidney cortex.

In the Phase 1 clinical study, REACT was administered via an 18-gauge needle. The proposed Phase 2 study utilizes an 18-gauge or smaller needle for REACT delivery. The needle is threaded inside the access cannula and advanced into the kidney, from which the REACT is administered. Injection of the REACT will be at a rate of 1 to 2 ml/min. After each 1 to 2 minute injection, the inner needle is retracted along the needle course within the cortex to the second site of injection, and so forth, until the needle tip reaches the end of the access cannula or until the entire REACT product has been injected. Using a percutaneous delivery approach, placement of the access cannula/trocar and delivery needle is performed using direct, real-time imaging. Options include ultrasound alone or ultrasound with complementary CT.

During the procedure, moderate conscious sedation is employed; vital signs are measured continuously. REACT injection ceases if there is imaging evidence of cell extravasation into central or peripheral renal blood vessels, the medullary portion of the kidney, or through the renal cortex and into the retroperitoneal soft tissues, or evidence of active bleeding.

Following completion of the REACT injection, the inner needle is withdrawn and the outer cannula remains in place for track embolization. During removal of the outer cannula (trocar), the site of the renal cortex puncture and needle track through the retroperitoneum are embolized with absorbable gelatin particle/pledgets (e.g., Gelfoam®[Pfizer]) or fibrin sealant (e.g., TISSEEL [Baxter]) or other suitable agent to prevent excessive renal bleeding.

Upon completion of the procedure, non-contrast CT scan or ultrasound with color Doppler evaluation is performed to image puncture site cell injection and any hematoma or bleeding events. The subject is monitored for 2 to 3 hours post-procedure in a recovery-room environment with nursing assessment and measurement of vital signs. Subjects who do not experience complications are discharged the same day as REACT injection, consistent with site standard practice.

### 8. Safety Assessments and Management

8.1. Adverse and Serious Adverse Events

8.1.1. Definition of Adverse Events

An AE is the development of an undesirable medical condition (including abnormal laboratory findings) or the deterioration of a pre-existing medical condition following or during exposure to a study treatment, whether or not considered to have a causal relationship with study procedures or the investigational product. A pre-existing condition is a clinical condition (including a condition being treated) that is diagnosed before the subject signs the Informed Consent Form and is documented as part of the subject's medical history. Pre-existing conditions that are stable or unchanged should not be considered adverse events.

The Investigator is responsible for ensuring that all AEs observed by the Investigator or reported by the subject that occur from the day of the biopsy procedure through 12 months after the final injection of REACT are monitored and recorded in the subject's medical record as well as the CRF provided by the Sponsor or its designee. AEs that occur from the time of consent and prior to the day of the biopsy procedure should be recorded as medical history for all subjects.

Treatment-emergent adverse events (TEAEs) are defined as any AE that started after the first injection of REACT, or started prior to the first injection but increased in severity or frequency after the first injection of REACT.

Unscheduled visits may be performed at any time during the study as judged necessary to assess and conduct follow-up on AEs.

8.1.1.1. Definition of Serious Adverse Events

A serious adverse event (SAE) is an adverse event that occurs during any phase of the study (i.e., baseline, treatment, washout, or follow-up) at any dose of the investigational product, comparator, or placebo, and fulfils one or more of the following:
Results in death
It is immediately life-threatening
It requires in-patient hospitalization or prolongation of existing hospitalization
It results in persistent or significant disability or incapacity
Results in a congenital abnormality or birth defect
It is an important medical event that may jeopardize the subject or may require medical intervention to prevent one of the outcomes listed above.

All SAEs that occur from the day of the biopsy procedure, during treatment, or within 12 months following the final REACT injection, whether or not they are related to study procedures or the investigational product, must be recorded in the subject's medical record as well as the CRF.

8.1.1.2. Other Significant Adverse Events

Significant events of particular clinical importance include SAEs and AEs leading to premature discontinuation of subjects from the study. These events are recorded in the subjects' medical records as well as the CRF. Narratives of these events may be prepared for inclusion in the Clinical Study Report.

The following sections describe “adverse events of special interest” concerning procedure- and product-related events. Subjects are carefully monitored for the occurrence of these potential AEs.

8.1.1.3. Procedure-Related Events

Post-procedure pain: If the subject experiences pain following the biopsy or REACT injection, administration of paracetamol or paracetamol-codeine combinations is recommended. More severe pain in the loin or abdomen requires ultrasonography to exclude significant perirenal hemorrhage. If severe pain occurs, administration of opiates may be necessary. If analgesic doses higher than the maximum authorized doses are required to alleviate pain, then the Investigator must perform additional clinical evaluations to ascertain the probable cause(s) of excessive pain.

Hemorrhage: Following renal biopsy and REACT injection procedures, subjects undergo regular hemoglobin and blood pressure monitoring. Subjects are confined to bed and monitored for maintenance of normal coagulation indices. If bleeding occurs and the subject is hypotensive despite bed rest, a blood transfusion may be considered. If the bleeding is still not controlled, surgery may be considered. In rare cases, renal angiography may be performed to identify the source of bleeding. Coil embolization can be performed during the same procedure.

Other complications: In very rare cases, other organs (such as liver, gallbladder and lungs) may be penetrated during the biopsy procedure. In these cases, appropriate treatment and follow-up may be discussed with consulting surgeons.

Death: Deaths resulting from renal biopsies occur in <0.01% of patients.[26, 34, 35] Adherence to strict inclusion/exclusion criteria ensures that subjects who may be predisposed to uncontrolled or excessive bleeding will not be enrolled in this trial.

8.1.1.4. Product-Related Events

No REACT product-related events are occur.

8.2. Adverse Event Intensity and Relationship Assessment

8.2.1. Intensity Scale

Intensity is assessed using the “Common Terminology Criteria for Adverse Events” (CTCAE) version 4.03, from the US National Cancer Institute (refer to evs.nci.nih.gov/ftpl/CTCAE/CTCAE_4.03_2010-06-14_QuickReference_8.5x11.pdf). If the AE is not included in the CTCAE, then the intensity of the AE is determined according to the following criteria:
Mild (Grade 1): The AE is noticeable to the subject but does not interfere with routine activity.
Moderate (Grade 2): The AE interferes with routine activity but responds to symptomatic therapy or rest.
Severe (Grade 3): The AE significantly limits the subject's ability to perform routine activities despite symptomatic therapy. Severe events are usually incapacitating.
Life-Threatening (Grade 4): The subject is at immediate risk of death.
Death (Grade 5)

If the intensity (grade) changes within a day, the maximum intensity (grade) is recorded. If the intensity (grade) changes over a longer period of time, the changes are recorded as separate events (having separate onset and stop dates for each grade).

It is important to distinguish between serious and severe AEs. Severity is a measure of intensity whereas seriousness is defined by the criteria under Definition of Serious Adverse Events (See Section 8.1.1.1). Therefore, an AE of severe intensity may not necessarily meet the criteria for seriousness.

8.2.2. Relationship Assessment

The Investigator should judge whether there is a reasonable possibility that the AE may have been caused by the study procedure or investigational product. If no valid reason exists for suggesting a relationship, then the AE is classified as “not related.” If there is any valid reason, even if undetermined, for suspecting a possible cause-and-effect relationship, then the AE is considered “possibly related” or “related” to the study procedure or investigational product.

Definitions of relatedness categories are:
Not Related: Exposure to the study treatment did not occur, or the occurrence of the AE is not reasonably related in time, or the AE is considered unlikely to be related to the study treatment.
Unlikely Related: The study treatment and the AE were not closely related in time, and/or the AE could be explained more consistently by causes other than exposure to the study treatment product.
Possibly Related: The study treatment and the AE were reasonably related in time, and the AE could be explained equally well by causes other than exposure to the study treatment product.
Related: The study treatment and the AE were reasonably related in time, and the AE is more likely explained by exposure to the study product than by other causes, or the study treatment was the most likely cause of the AE.

For the purpose of safety analyses, all AEs judged to be “possibly related” or “related” are considered treatment-related adverse events.

8.3. Recording and Reporting Adverse Events

Adverse events spontaneously reported by the subject and/or in response to an open question from study personnel, or revealed by observation, or documented via laboratory reports, imaging reports, consult notes, survey instruments and other data collection tools, are recorded in the subject's medical records and CRF.

An adverse event is reported using standard medical terminology, whenever possible. A clinically significant change in laboratory values or vital signs need not be reported as an AE unless the abnormal change constitutes an SAE and/or leads to discontinuation of treatment or withdrawal from the study.

For each AE, the start date, the stop date, the intensity of each reportable event, the judgment of the relationship to the study procedure or investigational product, the action taken, severity (if applicable), and whether the event resulted in discontinuation of treatment or withdrawal from the study is recorded.

8.3.1. Pregnancy

Pregnancy is neither an AE nor an SAE, unless a complication relating to the pregnancy occurs. All reports of congenital abnormalities/birth defects are SAEs. Spontaus miscarriages should be reported and handled as SAEs. However, elective abortions without complications should not be handled as AEs.

All pregnancies experienced by female subjects enrolled in this study are to be reported in the same time frame as SAEs using the Pregnancy Form of the CRF. The course of all pregnancies, including perinatal and natal outcome, regardless of whether the subject has discontinued participation in the study, are followed until resolution, including follow-up of the health status of the newborn to 6 weeks of age.

The effects of administration of the investigational product on the pregnant female or the developing fetus are unknown. Therefore, female subjects of child-bearing potential who are planning a pregnancy during the course of the study, or who are not using a highly effective method(s) of birth control, or who are unwilling to continue using a highly-effective method of birth control throughout the duration of the study are not eligible to participate in the study. (Refer to Exclusion Criterion #23; Section 5.2)

8.4. Stopping Rules for an Individual Subject

TA subject may be removed from the study for:
Any clinical adverse event, laboratory abnormality, intercurrent illness, other medical condition or situation whereby continued participation in the study would not be in the best interest of the subject.
Development of any exclusion criterion.

If a subject is terminated from the study, EOS assessments should be conducted at the last visit.

If any of the following events occur, no additional subjects can receive REACT injections until review has been completed:
An SAE that is rated as severe or life-threatening and is related to REACT or study procedures
Death of an enrolled subject
Similar SAE's in more than one subject that are related to REACT
Inability to deliver a minimum of 50% of the dose of REACT in more than one subject due to surgical or other issues

### 9. Statistical Methods and Planned Analyses

9.1. Sample Size

Up to 15 subjects who complete screening procedures and satisfy all inclusion and exclusion criteria are enrolled.

Statistical analyses are primarily descriptive in nature. Unless otherwise specified, continuous variables are summarized by presenting the number of non-missing observations (n), mean, standard deviation, median, minimum, and maximum. Categorical variables are summarized by presenting frequency count and percentage for each category.

9.2. Criteria for Evaluation

9.2.1. Analysis Objectives and Endpoints

9.2.1.1. Primary

To assess the safety of REACT injected in one recipient kidney.

Primary Endpoint: Procedure and/or product related adverse events (AEs) through 24 months post-injection.

9.2.1.2. Secondary

To assess the safety and tolerability of REACT administration by assessing renal-specific adverse events over a 24 month period following injection.

Secondary Endpoints: Renal-specific laboratory assessments through 24 months post injection.

9.2.1.3. Exploratory

To assess the impact of REACT on renal function over a 24 month period following injection.

Exploratory Endpoint: Clinical diagnostic and laboratory assessments of renal structure and function (including eGFR, serum creatinine, and proteinuria) to assess changes in the rate of progression of renal disease.

9.3. Demographic and Baseline Characteristics

Demographic data and baseline characteristics are summarized via sample size, mean, standard deviation, median, minimum, and maximum for the continuous variables as well as the frequency and proportion for categorical variables. These summaries are produced for both the full analysis set and the injection analysis set. Demographic and baseline characteristic information are presented as descriptive statistics; generating inferential statistics is not planned. These data are provided in a tabular listing.

9.4. Efficacy Analysis

The primary efficacy endpoint is serial measurements of eGFR obtained at 1, 3, 6, and 12 months after the last REACT injection. GFR are estimated using the Chronic Kidney Disease Epidemiology Collaboration (CKD-EPI) equation that incorporates both serum creatinine and Cystatin C. [1]

Estimated GFR measured at each time point are summarized by presenting descriptive statistics of raw data and change from baseline values for each treatment group.

9.5. Exploratory Analysis

An exploratory analysis is conducted to examine potential changes in health-related Quality of Life (HR-QoL).

9.6. Statistical Methods

Subjects complete the KDQOL-SF™ survey (i.e., Kidney Disease and Quality of Life Short Form). The KDQOL-SF is a 36-item, validated, HR-QoL instrument relevant to patients with kidney disease.[42] This disease-specific, HR-QoL instrument consists of the following subscales:
The “SF-12 measure of physical (PCS) and mental (MCS) functioning” contains items about general health, activity limits, ability to accomplish desired tasks, depression and anxiety, energy level, and social activities.
The “Burden of Kidney Disease subscale” contains items about how kidney disease interferes with daily life, takes up time, causes frustration, or makes the respondent feel like a burden.
The “Symptoms and Problems subscale” contains items about how bothered a respondent feels by sore muscles, chest pain, cramps, itchy or dry skin, shortness of breath, faintness/dizziness, lack of appetite, feeling washed out or drained, numbness in the hands or feet, nausea, or problems with dialysis access.
The “Effects of Kidney Disease on Daily Life subscale” contains items about how bothered the respondent feels by fluid limits, diet restrictions, ability to work around the house or travel, feeling dependent on doctors and other medical staff, stress or worries, sex life, and personal appearance.

9.7. Safety Analysis

9.8 Laboratory Evaluations

Baseline values are collected immediately prior to REACT injection. Observed and change from baseline laboratory data is summarized via sample size, mean, standard deviation, median, minimum, and maximum for the continuous variables as well as frequency and proportion for the categorical variables. Laboratory abnormalities are defined using the NCI CTCAE grading scheme. Abnormal laboratory values are flagged as above or below the normal range. The results of laboratory testing for renal function, specifically sCr, BUN, and urinary albumin, are of particular interest for this study.

The incidence of treatment-emergent laboratory abnormalities, defined as values that increase at least one toxicity grade between baseline and any time post-baseline up to six months following REACT injection, are summarized. If baseline data are missing, then the latest value between biopsy and injection is used as the baseline value. If baseline and pre-injection data are missing, then any graded abnormality (i.e., at least a Grade 1) is considered as treatment-emergent. These values are summarized for both the full analysis set and the injection analysis set. Observed and change from baseline values are presented as descriptive statistics; generating inferential statistics is not planned. These data are provided in a tabular listing.

9.9. Adverse Events

Clinical and laboratory AEs are coded using the Medical Dictionary for Regulatory Activities (MedDRA) version 18.1 by System Organ Class (SOC) and Preferred Term (PT). Adverse events are graded using the CTCAE version 4.03 from the US National Cancer Institute.

A treatment-emergent AE is defined as any adverse event that started after the first injection of REACT, or started prior to the first injection but increased in severity or frequency after the first injection of REACT. Summaries (frequency and proportion) of treatment-emergent AE's are presented by SOC and PT. Additional summaries include, but are not limited to, treatment-emergent AE judged to be related to the procedure and/or investigational product, intensity, reason for subject withdrawal, SAEs, and deaths. The numbers of events (occurrence) and the number of subjects (incidence) who experienced treatment-emergent AEs are reported by treatment group. Adverse event data is provided in a data listing.

9.10. Other Safety Evaluations

Change from baseline for vital signs are calculated for each subject and provided in a data listing. The number and percent of subjects who exhibit change(s) in their physical examinations (such as from normal to abnormal) are summarized via a data listing. The number and percent of subjects who develop abnormal heart rhythms or QT-interval prolongation during the study are provided in a data listing. Data from medical history, concomitant medications, ultrasound, renal scintigraphy, and MRI assessments are provided in a data listing. Descriptive statistics for these evaluations are generated as warranted.

9.11. Biopsy and REACT Injection(s)

Biopsy and REACT injection data is provided in a data listing.

### 10. Results

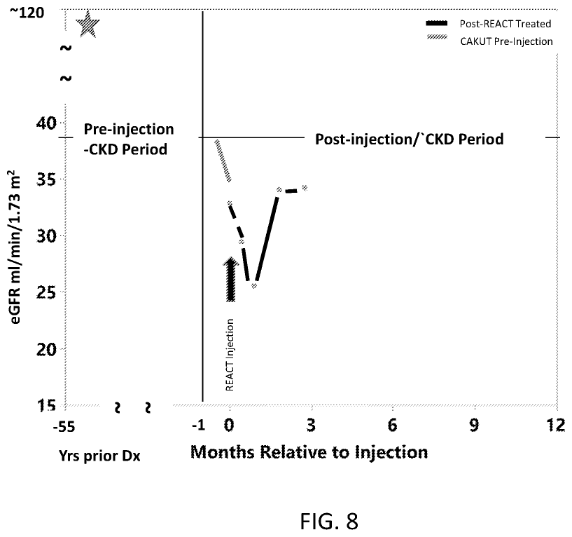

A patient having kidney disease resulting from CAKUT was injected with REACT. The patient was a 55-year old male having a posterior urethral valve. At three months post-REACT injection, the most recent time point for which data was available, the patient exhibited detectable improvements in kidney function as demonstrated by an increase in eGFR and decrease in abumin to creatinine ratio (ACR). As shown in FIG. 8, injection of the patient with REACT improved, i.e., increased, the patient's renal function as measured by eGFR. In the month preceding REACT injection, the patient's eGFR had been in decline, declining from approximately 40 mL/min/1.73 m², 1 month prior to injection, to approximately 33 mL/min/1.73 m², at the time of injection (FIG. 8, solid gray line). Injection of the patient with REACT increased the patient's eGRF, relative to its at-time-of-injection value, to approximately 34 mL/min/1.73 m² 2 months post-injection and approximately 35 mL/min/1.73 m² 3 months post injection (FIG. 8, broken black line).

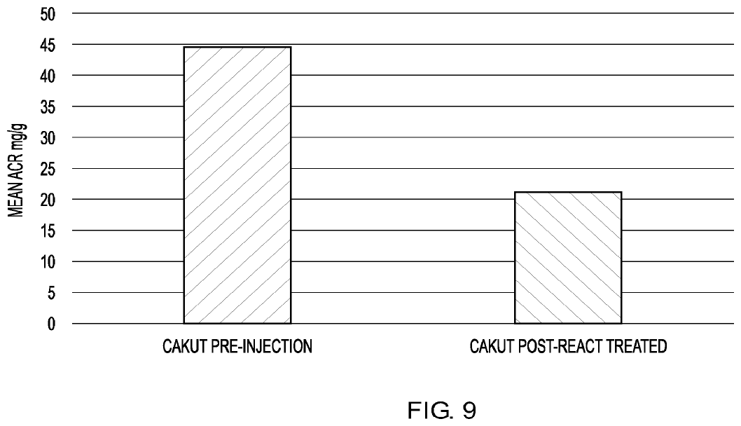

FIG. 9 provides further evidence of the patient's improved kidney function via an observed decrease in the patient's Albumin-to-Creatinine Ratio (ACR). At the time of REACT injection, the patient's ACR was elevated, at a level of approximately 47 mg/g. However, 3 months following REACT injection the patient's ACR was reduced by over 50%, to approximately 21 mg/g, which can be considered an ACR value in the “normal”, i.e., below 30 mg/g, range.

These observations of improved kidney function post-REACT injection are clinically significant; current therapeutic options to treat CKD, regardless of underlying cause, are unable to improve kidney function and, rather, merely reduce the rate of decline of the organ's function.

### REFERENCES

1. Lees, G. E., R. E. Cianciolo, and F. J. Clubb, Jr., Renal biopsy and pathologic evaluation of glomerular disease. Top Companion Anim Med, 2011. 26(3): p. 143-53.
2. Nass, K. and W. C. O'Neill, Bedside renal biopsy: ultrasound guidance by the nephrologist.

Am J Kidney Dis, 1999. 34(5): p. 955-9.
3. Oberholzer, M., et al., Minimum sample size of kidney biopsies for semiquantitative and quantitative evaluation. Nephron, 1983. 34(3): p. 192-5.
4. Dhingra, R. K., et al., Type of vascular access and mortality in U.S. hemodialysis patients. Kidney Int, 2001. 60(4): p. 1443-51.
5. USRDS, 2012 Annual Data Report: Atlas of Chronic Kidney Disease and End-Stage Renal Disease in the United States. National Institutes of Health, National Institute of Diabetes and Digestive and Kidney Diseases, Bethesda, Md., 2012.
6. Almeida, C. C., et al., Safety of immunosuppressive drugs used as maintenance therapy in kidney transplantation: a systematic review and meta-analysis. Pharmaceuticals (Basel), 2013. 6(10): p. 1170-94.
7. Atala, A., et al., Tissue-engineered autologous bladders for patients needing cystoplasty. Lancet, 2006. 367(9518): p. 1241-6.
8. Bruce, A. T., et al., Exposure of Cultured Human Renal Cells Induces Mediators of cell migration and attachment and facilitates the repair of tubular cell monolayers in vitro. Experimental Biology, Washington, D C, 2011.
9. Bruce, A. T., et al., Selected renal cells modulate disease progression in rodent models of chronic kidney disease via NF-kappaB and TGF-beta1 pathways. Regen Med, 2015. 10(7): p. 815-39.
10. Jayo, M. J., et al., Long-term durability, tissue regeneration and neo-organ growth during skeletal maturation with a neo-bladder augmentation construct. Regen Med, 2008. 3(5): p. 671-82.
11. Jayo, M. J., et al., Early cellular and stromal responses in regeneration versus repair of a mammalian bladder using autologous cell and biodegradable scaffold technologies. J Urol, 2008. 180(1): p. 392-7.
12. Jayo, M. J., et al., Tissue engineering and regenerative medicine: role of toxicologic pathologists for an emerging medical technology. Toxicol Pathol, 2008. 36(1): p. 92-6.
13. Bilan, V. P., et al., Diabetic nephropathy and long-term treatment effects of rosiglitazone and enalapril in obese ZSF1 rats. J Endocrinol, 2011. 210(3): p. 293-308.
14. Ilagan, R., et al., Exosomes derived from primary renal cells contain microRNAs that can potentially drive therapeutically-relevant outcomes in models of chronic kidney disease. TERMIS Conference, Orlando, Fla., 2010.
15. Ilagan, R., et al., Secreted Factors from Bioactive Kidney Cells Attenuate NF-kappa-B. TERMIS Conference, Orlando, Fla., 2010.
16. Kelley, R., et al., A Population of Selected Renal Cells Augments Renal Function and Extends Survival in the ZSF1 model of Progressive Diabetic Nephropathy Cell Transplantation (in Press), 2012.
17. Kelley, R., et al., Bioactive Renal Cells Augment Kidney Function In a Rodent Model Of Chronic Kidney Disease. ISCT Conference, Philadelphia, Pa., 2010.
18. Brenner, B. M., Nephron adaptation to renal injury or ablation. Am J Physiol, 1985. 249(3 Pt 2): p. F324-37.
19. Montgomery, A. V., et al., The intrarenal pressure; its relation to age, weight, blood pressure, and sex. J Exp Med, 1950. 92(6): p. 637-42.
20. Swann, H. G., et al., The intrarenal pressure during experimental renal hypertension. J Exp Med, 1952. 95(5): p. 281-91.
21. Kelley, R. W., et al., Enhanced renal cell function in dynamic 3D culture system. KIDSTEM Conference, Liverpool, U K, 2008.
22. Kelley, R., et al., Intra-renal Transplantation of Bioactive Renal Cells Preserves Renal Functions and Extends Survival in the ZSF1 model of Progressive Diabetic Nephropathy. ADA Conference, San Diego, Calif., 2011.
23. Basu J, S. N., Rivera E, Guthrie K, Bertram T, Jain D. Dynamic distribution of therapeutically bioactive selected renal cells in large animal pre-clinical model. in International Society for Stem Cell Research. 2014. Vancouver, BC.
24. Hare, J. M., et al., Comparison of allogeneic vs autologous bone marrow-derived mesenchymal stem cells delivered by transendocardial injection in patients with ischemic cardiomyopathy: the POSEIDON randomized trial. JAMA, 2012. 308(22): p. 2369-79.
25. Hare, J. M., et al., A randomized, double-blind, placebo-controlled, dose-escalation study of intravenous adult human mesenchymal stem cells (prochymal) after acute myocardial infarction. J Am Coll Cardiol, 2009. 54(24): p. 2277-86.
26. Heldman, A. W., et al., Transendocardial mesenchymal stem cells and mononuclear bone marrow cells for ischemic cardiomyopathy: the TAC-HFT randomized trial. JAMA, 2014. 311(1): p. 62-73.
27. Lee, J. S., et al., A long-term follow-up study of intravenous autologous mesenchymal stem cell transplantation in patients with ischemic stroke. Stem Cells, 2010. 28(6): p. 1099-106.
28. Malliaras, K., et al., Intracoronary cardiosphere-derived cells after myocardial infarction: evidence of therapeutic regeneration in the final 1-year results of the CADUCEUS trial (CArdiosphere-Derived aUtologous stem CElls to reverse ventricUlar dySfunction). J Am Coll Cardiol, 2014. 63(2): p. 110-22.
29. Babitt, J. L. and H. Y. Lin, Mechanisms of anemia in CKD. J Am Soc Nephrol, 2012. 23(10): p. 1631-4.
30. Khajehdehi, P., et al., Percutaneous renal biopsy in the 1990s: safety, value, and implications for early hospital discharge. Am J Kidney Dis, 1999. 34(1): p. 92-7.
31. Manno, C., et al., Desmopressin acetate in percutaneous ultrasound-guided kidney biopsy: a randomized controlled trial. Am J Kidney Dis, 2011. 57(6): p. 850-5.
32. Groman, R. P., et al., Effects of serial ultrasound-guided renal biopsies on kidneys of healthy adolescent dogs. Vet Radiol Ultrasound, 2004. 45(1): p. 62-9.
33. Hergesell, O., et al., Safety of ultrasound-guided percutaneous renal biopsy-retrospective analysis of 1090 consecutive cases. Nephrol Dial Transplant, 1998. 13(4): p. 975-7.
34. Lin, W. C., et al., Outpatient versus inpatient renal biopsy: a retrospective study. Clin Nephrol, 2006. 66(1): p. 17-24.
35. Walker, P. D., The renal biopsy. Arch Pathol Lab Med, 2009. 133(2): p. 181-8.
36. Medarova, Z., et al., In vivo imaging of autologous islet grafts in the liver and under the kidney capsule in non-human primates. Transplantation, 2009. 87(11): p. 1659-66.
37. Park, H. C., et al., Renal capsule as a stem cell niche. Am J Physiol Renal Physiol, 2010. 298(5): p. F1254-62.
38. Salagierski, M. and M. S. Salagierski, Radiofrequency ablation: a minimally invasive approach in kidney tumor management. Cancers (Basel), 2010. 2(4): p. 1895-900.
39. Cain, H., E. Egner, and M. Redenbacher, Increase of mitosis in the tubular epithelium following intrarenal doses of various kidney homogenates and hemogenate fractions in the rat. Virchows Arch B Cell Pathol, 1976. 22(1): p. 55-72.
40. Humphreys, B. D., et al., Intrinsic epithelial cells repair the kidney after injury. Cell Stem Cell, 2008. 2(3): p. 284-91.
41. Inker, L. A., et al., Estimating glomerular filtration rate from serum creatinine and cystatin C. N Engl J Med, 2012. 367(1): p. 20-9.
42. Hays R D, K. J., Mapes D L. Kidney Disease Quality of Life Short Form KDQOL-SF) Version 1.3. A Manual for Use and Scoring. RAND; Santa Monica, Calif.: 1995. www.rand.org/health/surveys_tools/kdqol.html. Accessed 28 Apr. 2016.

### Example 2—Non-Limiting Examples of Methods and Compositions for Producing SRCs

### Example 2.1—Preparation of Solutions

This example section provides the compositions of the various media formulations and solutions used for the isolation and characterization of the heterogeneous renal cell population, and manufacture of the regenerative therapy product, in this example.

TABLE 13
Culture Media and Solutions

| Material | Composition |
|---|---|
| Tissue Transport | Viaspan ™ or HypoThermosol-FRS ® or DMEM |
| Medium | Kanamycin: 100 μg/mL |
| Renal Cell Growth | DMEM:KSFM (50:50) |
| Medium | 5% FBS |
| | Growth Supplements: |
| | HGF: 10 mg/L |
| | EGF: 2.5 μg/L |
| | Insulin: 10.0 mg/L |
| | Transferrin: 5.5 mg/L |
| | Selenium: 670 μg/L |
| | Kanamycin: 10 μg/L |
| Tissue Wash | DMEM |
| Solution | Kanamycin: 10 μg/mL |
| Digestion Solution | Collagenase IV: 300 Units |
| | Dispase: 5 mg/mL |
| | Calcium Chloride: 5 mM |
| Cell Dissociation | TrypLE ™ |
| Solution | |
| Density Gradient | 7% OptiPrep |
| Solution | OptiMEM |
| Cryopreservation | DMEM or HypoThermosol—FRS |
| Solution | 10% DMSO |
| | 10% FBS |

Dulbecco's Phosphate Buffered Saline (DPBS) was used for all cell washes.

### Example 2.2—Isolation of the Heterogeneous Unfractionated Renal Cell Population

This example section illustrates the isolation of an unfractionated (UNFX) heterogeneous renal cell population from human. Initial tissue dissociation was performed to generate heterogeneous cell suspensions from human kidney tissue.

Renal tissue via kidney biopsy provided the source material for a heterogeneous renal cell population. Renal tissue comprising one or more of cortical, corticomedullary junction or medullary tissue may be used. In one embodiment, the corticomedullary junction tissue is used. Multiple biopsy cores (minimum 2), avoiding scar tissue, were required from a CKD kidney. Renal tissue was obtained by the clinical investigator from the patient at the clinical site approximately 4 weeks in advance of planned implantation of the final NKA. The tissue was transported in the Tissue Transport Medium of Example 2.1.

The tissue was then washed with Tissue Wash Solution of Example 2.1 in order to reduce incoming bioburden before processing the tissue for cell extractions.

Renal tissue was minced, weighed, and dissociated in the Digestion Solution of Example 2.1. The resulting cell suspension was neutralized in Dulbecco's Modified Eagle Medium (D-MEM)+10% fetal bovine serum (FBS) (Invitrogen, Carlsbad Calif.), washed, and resuspended in serum-free, supplement-free, Keratinocyte Media (KSFM) (Invitrogen). Cell suspensions were then subjected to a 15% (w/v) iodixanol (OptiPrep™, Sigma) gradient to remove red blood cells and debris prior to initiation of culture onto tissue culture treated polystyrene flasks or dishes at a density of 25,000 cells per cm² in Renal Cell Growth Medium of Example 2.1. For example, cells may be plated onto T500 Nunc flask at 25×10⁶ cells/flask in 150 ml of 50:50 media.

### Example 2.3—Cell Expansion of the Isolated Renal Cell Population

Renal cell expansion is dependent on the amount of tissue received and on the success of isolating renal cells from the incoming tissue. Isolated cells can be cryopreserved, if required (see infra). Renal cell growth kinetics may vary from sample to sample due to the inherent variability of cells isolated from individual patients.

A defined cell expansion process was developed that accommodates the range of cell recoveries resulting from the variability of incoming tissue Table 14. Expansion of renal cells involved serial passages in closed culture vessels (e.g., T-flasks, Cell Factories, HyperStacks®) in Renal Cell Growth Medium Table 13 using defined cell culture procedures.

A BPE-free medium was developed for human clinical trials to eliminate the inherent risks associated with the use of BPE. Cell growth, phenotype (CK18) and cell function (GGT and LAP enzymatic activity) were evaluated in BPE-free medium and compared to BPE containing medium used in the animal studies. Renal cell growth, phenotype and function were equivalent in the two media. (data not shown)

TABLE 14
Cell Recovery from Human Kidney Biopsies

| | Renal cells (cells/10 mg tissue) | |
|---|---|---|
| Source | (passage 0) | (passage 1) |
| Human Kidney Tissue | 1.44 − 10.80 × 10⁶ | 4.61 − 23.10 × 10⁷ |
| Samples (n = 7) | | |

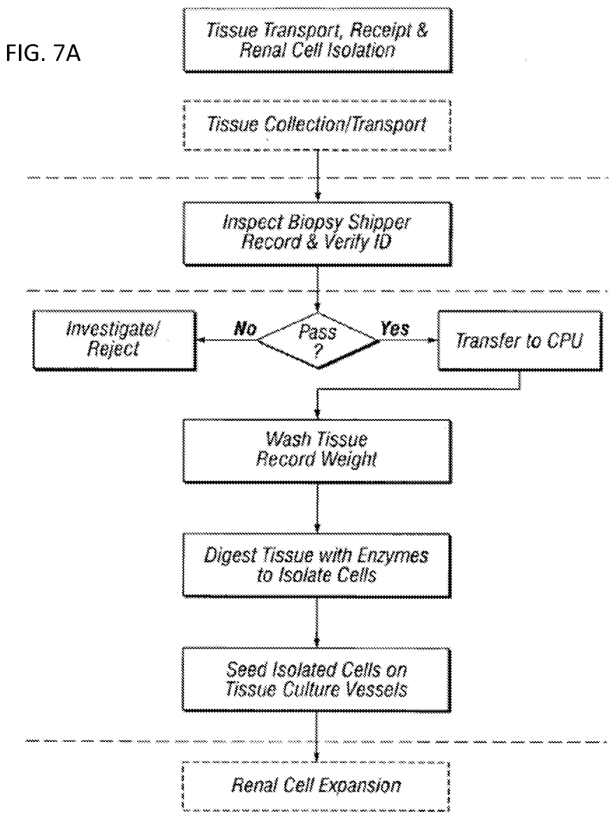

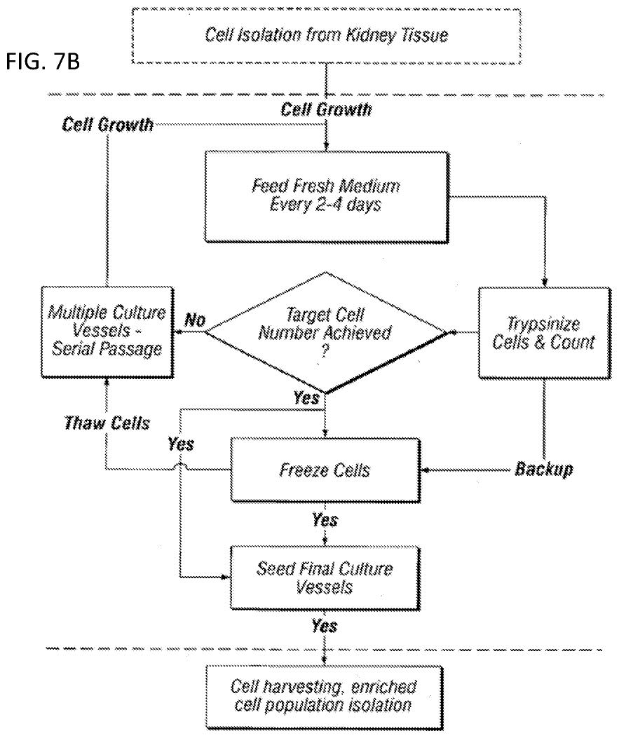

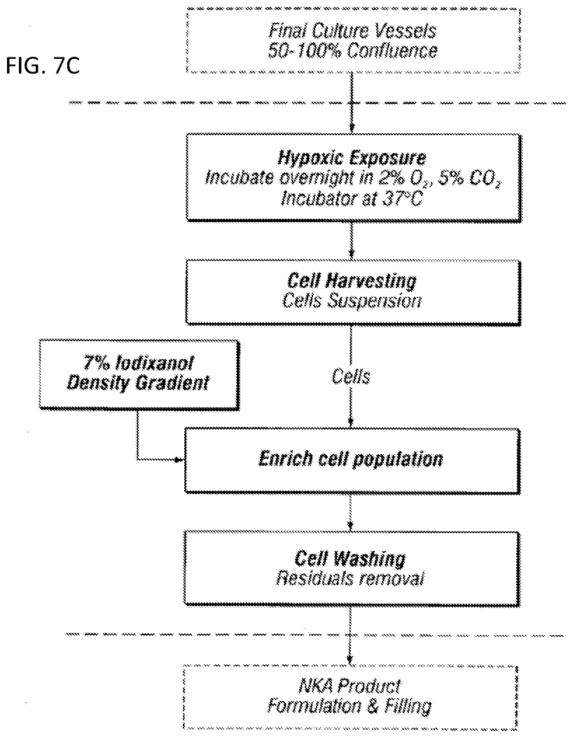

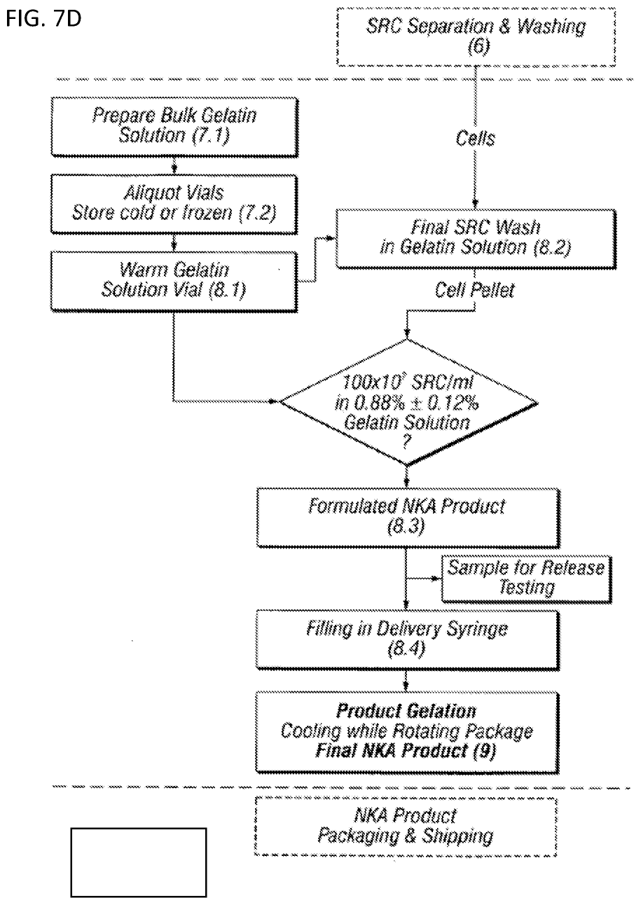

Once cell growth was observed in the initial T-flasks (passage 0) and there were no visual signs of contamination, culture medium was replaced and changed thereafter every 2-4 days (FIG. 7B). Cells were assessed to verify renal cell morphology by visual observation of cultures under the microscope. Cultures characteristically demonstrated a tight pavement or cobblestone appearance, due to the cells clustering together. These morphological characteristics vary during expansion and may not be present at every passage. Cell culture confluence was estimated using an Image Library of cells at various levels of confluence in the culture vessels employed throughout cell expansions.

Renal cells were passaged by trypsinization when culture vessels are at least 50% confluent (FIG. 7B). Detached cells were collected into vessels containing Renal Cell Growth Medium, counted and cell viability calculated. At each cell passage, cells were seeded at 500-4000 cells/cm² in a sufficient number of culture vessels in order to expand the cell number to that required for formulation of NKA (FIG. 7B). Culture vessels were placed in a 37° C. incubator in a 5% CO2 environment. As described above, cell morphology and confluence was monitored and tissue culture media was replaced every 2-4 days. Table 15 lists the viability of human renal cells observed during cell isolation and expansion of six kidney biopsies from human donors.

TABLE 15
Cell Viability of Human Renal Cells in Culture

| Passage (n = 6) | Cell Viability (Average %) | Range (%) |
|---|---|---|
| P0 | 88 | 84-93 |
| P1 | 91 | 80-98 |
| P2 | 94 | 92-99 |
| P3 | 98 | 97-99 |

Inherent variability of tissue from different patients resulted in different cell yield in culture. Therefore, it was not practical to strictly define the timing of cell passages or number and type of culture vessels required at each passage to attain target cell numbers. Typically renal cells undergo 2 or 3 passages; however, duration of culture and cell yield can vary depending on the cell growth rate.

Cells were detached for harvest or passage with 0.25% Trypsin with EDTA (Invitrogen). Viability was assessed via Trypan Blue exclusion and enumeration was performed manually using a hemacytometer or using the automated Cellometer® counting system (Nexcelom Bioscience, Lawrence Mass.).

### Example 2.4 Cryopreservation of Cultured Cells

Expanded renal cells were routinely cryopreserved to accommodate for inherent variability of cell growth from individual patients and to deliver product on a pre-determined clinical schedule. Cryopreserved cells also provided a backup source of cells in the event that another NKA was needed (e.g., delay due to patient sickness, unforeseen process events, etc.). Conditions were established that have been used to cryopreserve cells and recover viable, functional cells upon thawing.

For cryopreservation, cells were suspended to a final concentration of about 50×10⁶ cells/mL in Cryopreservation Solution (see Example 2.1) and dispensed into vials. One ml vials containing about 50×10⁶ cells/mL were placed in the freezing chamber of a controlled rate freezer and frozen at a pre-programmed rate. After freezing, the cells were transferred to a liquid nitrogen freezer for in-process storage.

### Example 2.5 Preparation of SRC Cell Population

Selected Renal Cells (SRC) can be prepared from the final culture vessels that have grown from cryopreserved cells or directly from expansion cultures depending on scheduling (FIG. 7B).

If using cryopreserved cells, the cells were thawed and plated on tissue culture vessels for one final expansion step. When the final culture vessels were approximately 50-100% confluent cells were ready for processing for SRC separation. Media exchanges and final washes of NKA dilute any residual Cryopreservation Solution in the final product.

Once the final cell culture vessels had reached at least 50% confluence the culture vessels were transferred to a hypoxic incubator set for 2% oxygen in a 5% CO2 environment at 37° C. (FIG. 7C) and cultured overnight. Cells may be held in the oxygen-controlled incubator set to 2% oxygen for as long as 48 hours. Exposure to the more physiologically relevant low-oxygen (2%) environment improved cell separation efficiency and enabled greater detection of hypoxia-induced markers such as VEGF.

After the cells had been exposed to the hypoxic conditions for a sufficient time (e.g., overnight to 48 hours), the cells were detached with 0.25% Trypsin with EDTA (Invitrogen). Viability was assessed via Trypan Blue exclusion and enumeration was performed manually using a hemacytometer or using the automated Cellometer® counting system (Nexcelom Bioscience, Lawrence Mass.). Cells were washed once with DPBS and resuspended to about 850×10⁶ cells/mL in DPBS.

Density gradient centrifugation was used to separate harvested renal cell populations based on cell buoyant density. Renal cell suspensions were separated on single-step 7% iodixanol Density Gradient Solution (OptiPrep; 60% (w/v) in OptiMEM; see Example 2.1).

The 7% OptiPrep gradient solution was prepared and refractive index indicative of desired density was measured (R.I. 1.3456+/−0.0004) prior to use. Harvested renal cells were layered on top of the gradient solution. The density gradient was centrifuged at 800 g for 20 min at room temperature (without brake) in either centrifuge tubes or a cell processor (e.g., COBE 2991). The cellular fraction exhibiting buoyant density greater than approximately 1.045 g/mL was collected after centrifugation as a distinct pellet. Cells maintaining a buoyant density of less than 1.045 g/mL were excluded and discarded.

The SRC pellet was re-suspended in DPBS (FIG. 7C). The carry-over of residual OptiPrep, FBS, culture medium and ancillary materials in the final product is minimized by 4 DPBS wash and 1 Gelatin Solution steps.

What is claimed is:

1. A method of treating kidney disease in a subject who has chronic kidney disease (CKD), the method comprising administering to the subject an effective amount of a composition comprising:
(i) a bioactive renal cell population;
(ii) vesicles secreted by the renal cell population; and/or
(iii) spheroids comprising the renal cell population and at least one non-renal cell population,
wherein the subject has an anomaly of a kidney and/or urinary tract.

2. The method of claim 1, wherein the subject has CKD from congenital anomalies of the kidney and urinary tract (CAKUT).

3. The method of claim 1 or 2, wherein the subject has anomolies in kidney development

4. The method of any one of claims 1 to 3, wherein the subject has or has had primary or secondary vesicoureteral reflux, reflux nephropathy, renal scaring, or renal hypodysplasia with or without infection and/or inflammation.

5. The method of any one of claims 1 to 4, wherein the subject is predisposed to urinary tract infections.

6. The method of any of any one of claims 1 to 5, wherein the subject has hypertension or proteinuria.

7. The method of any one of claims 1 to 6, wherein the subject has had post-antireflux surgery.

8. The method of any one of claims 1 to 7, wherein the subject has a glomerular filtration rate (GFR) of less than 90 mL/min/1.73 m², microalbuminuria, or macroalbuminuria.

9. The method of any one of claims 1 to 8, wherein the subject is less than 18 years old.

10. The method of any one of claims 1 to 9, wherein the subject has a Renal Parenchymal Malformation.

11. The method of any one of claims 1 to 10, wherein the subject has a ureteral duplication, a ureteropelvic junction obstruction, renal agenesis, vesicoureteral reflux, renal dysplasia, renal hypoplasia, renal hypodysplasia, congenital hydronephrosis, a horseshoe kidney, posterior urethral valve and prune belly syndrome, obstructive renal dysplasia, or a nonmotile ciliopathy.

12. The method of any one of claims 2 to 11, wherein the CAKUT has been caused by or has been correlated with a genetic factor.

13. The method of any one of claims 2 to 11, wherein the CAKUT has been caused by or has been correlated with a non-genetic factor.

14. The method of claim 13, wherein the non-genetic factor is an environmental factor.

15. The method of any one of claims 1 to 14, wherein the anomaly comprises Alagille syndrome, Apert syndrome, Bardet-Biedl syndrome, Beckwith-Wiedemann syndrome, Branchio-Oto-Renal syndrome (BOR), Campomelic dysplasia, Cenani-Lenz syndrome, DiGeorge syndrome, Fraser syndrome, hypoparathyroidism sensorineural deafness and renal anomalies (HDR), Kallmann syndrome, Mammary-Ulnar syndrome, Meckel Gruber syndrome, nephronophthisis, Okihiro syndrome, Pallister-Hall syndrome, Renal coloboma syndrome, hypoplasia, dysplasia, renal dysplasia, cystic dysplasia, non-cystic dysplasia, VUR Cystic dysplasia, renal hypoplasia, isolated cystic renal hypoplasia, isolated non-cystic renal hypoplasia, isolated renal tubular dysgenesis, Rubinstein-Taybi syndrome, Simpson-Golabi Behmel syndrome, Townes-Brock syndrome, Zellweger syndrome, Smith-Lemli-Opitz syndrome, hydronephrosis, medullary dysplasia, unilateral/bilateral agenesis/dysplasia, collecting system anomalies, agenesis, ureteropelvic junction obstruction (UPJO) agenesis, dysplasia agenesis, unilateral agenesis, VUR, malrotation, cross-fused ectopia, VUR Dysplasia, a dual Serine/Threonine And Tyrosine Protein Kinase (DSTYK) mutation, a DSTYK mutation associated with UPJO, tubular dysgenesis, cysts, and/or aplasia.

16. The method of any one of claims 1 to 15, wherein the subject has end-stage kidney disease.

17. The method of any one of claims 1 to 16, wherein the chronic kidney disease is Stage I, II, III, IV, or V kidney disease.

18. The method of any one of claims 1 to 17, wherein the subject is receiving dialysis at least 1, 2, or 3 times per week.

19. The method of any one of claims 1 to 18, wherein at least greater than 80% of the cells in the bioactive renal cell population express GGT-1.

20. The method of any one of claims 1 to 19, wherein 4.5% to 81.2% of the cells in the bioactive renal cell population express GGT-1, 3.0% to 53.7% of the cells within the bioactive renal cell population express AQP2, and 81.1% to 99.7% of the cells within the bioactive renal cell population express CK18.

21. The method of any one of claims 1 to 20, wherein the bioactive renal cell population is enriched for renal tubular cells compared to a primary culture of kidney cells from a kidney biopsy, and the tubular cells express higher molecular weight species of hyaluronic acid (HA) both in vitro and in vivo, through the actions of hyaluronic acid synthase-2 (HAS-2).

22. The method of any one of claims 1 to 21, wherein the bioactive renal cell population has a lesser proportion of distal tubular cells, collecting duct cells, endocrine cells, vascular cells, and/or progenitor-like cells compared to a primary culture of kidney cells from a kidney biopsy.

23. The method of any one of claims 1 to 22, wherein the vesicles comprise a paracrine factor.

24. The method of any one of claims 1 to 23, wherein the vesicles comprise an miRNA that inhibits Plasminogen Activation Inhibitor-1 (PAI-1) and/or TGFβ1.

25. The method of any one of claims 1 to 24, wherein the at least one non-renal cell population is an endothelial cell population or an endothelial progenitor cell population.

26. The method of any one of claims 1 to 24, wherein the at least one non-renal cell population is a mesenchymal stem cell population.

27. The method of any one of claims 1 to 26, wherein the administering is by injection into one or both kidneys of the subject.

28. The method of any one of claims 1 to 27, wherein the composition further comprises a temperature-sensitive cell-stabilizing biomaterial that maintains (i) a substantially solid state at 8° C. or below, and (ii) a substantially liquid state at ambient temperature or above, wherein the biomaterial comprises a hydrogel, wherein the biomaterial is in a solid-to-liquid transitional stage between 8° C. and ambient temperature or above.

29. The method of claim 28, wherein the bioactive renal cell population, the vesicles, and/or the spheroids are suspended in and dispersed throughout the cell-stabilizing biomaterial.

30. The method of claim 28 or 29, wherein the hydrogel comprises gelatin.

31. The method of any one of claims 1 to 30, wherein the bioactive renal cell population, the vesicles, and/or the spheroids are administered by injection through a 18 to 30 gauge needle.

32. The method of claim 31, wherein the needle has a diameter of about 27 gauge, about 26 gauge, about 25 gauge, about 24 gauge, about 23 gauge, about 22 gauge, about 21 gauge, or about 20 gauge.

33. The method of any one of claims 1-17, wherein the treating the kidney disease comprises improving renal function of the subject.

34. The method of claim 33, wherein the improving renal function comprises reducing albumin-to creatinine ratio (ACR) in the subject.

35. The method of claim 34, wherein the reducing ACR is by at least 50% relative to baseline ACR of the subject.

36. The method of claim 35, wherein the reducing ACR is by at least 60% relative to baseline ACR of the subject.

37. The method of claim 34, wherein the reducing the ACR is a reduction in ACR to between 30 mg/g and 300 mg/g, wherein subject comprises an ACR of greater than 300 mg/g prior to the administering a first dose of the composition.

38. The method of claim 34, wherein the reducing the ACR is a reduction in ACR to less than 30 mg/g, wherein the subject comprises an ACR of between 30 mg/g and 300 mg/g prior to the administering a first dose of the composition.

39. The method of any of claims 34-36, wherein the reducing ACR is achieved within 3-6 months following the administering a first dose of the composition.

40. The method of any of claims 34-36, wherein the reducing ACR is achieved within 2-3 months following the administering a first dose of the composition.

41. The method of claim 33, wherein the improving renal function comprises increasing eGFR of the subject.

42. The method of 41, wherein the increasing eGFR is achieved within two to four months following the administering a first dose of the composition.

43. The method of claim 41, wherein the increasing eGFR is achieved within two months following the administering a first dose of the composition.

44. The method of any of claims 41-43, wherein the increase in eGFR is at least 5% over baseline eGFR of the subject.

45. The method of claim 44, wherein the increase in eGFR is at least 10% over baseline eGFR of the subject.

46. The method of any of claims 33-45, wherein the anomaly of the kidney and/or urinary tract comprises a posterior urethral valve.

47. The method of any of claims 1-46, wherein the composition comprises the (i) bioactive renal cell population.

48. The method of claim 47, wherein the effective amount of the bioactive renal cell population comprises 3×10⁶ cells/gram estimated kidney weight of the subject.
